(19) United States
(12) Patent Application Publication
(10) Pub. No.: US 2022/0047672 A1
(43) Pub. Date: Feb. 17, 2022

(54) COMPOSITIONS AND METHODS INVOLVING LAYILIN

(71) Applicant: The Regents of the University of California, Oakland, CA (US)

(72) Inventors: Michael D. Rosenblum, San Francisco, CA (US); Kelly M. Mahuron, San Francisco, CA (US); Joshua M. Moreau, San Francisco, CA (US); Mariela Pauli, San Francisco, CA (US); Pooja Mehta, San Francisco, CA (US)

(21) Appl. No.: 17/396,475

(22) Filed: Aug. 6, 2021

Related U.S. Application Data
(63) Continuation of application No. PCT/US2020/017557, filed on Feb. 10, 2020.
(60) Provisional application No. 62/802,855, filed on Feb. 8, 2019.
(60) Provisional application No. 62/880,022, filed on Jul. 29, 2019.

(51) Int. Cl. A61K 38/17; C07K 16/18; A61K 35/17; A61P 35/00; A61P 37/06

(52) U.S. Cl. CPC A61K 38/1709; C07K 16/18; A61K 35/17; A61K 39/00; A61P 37/06; C07K 2317/31; A61P 35/00

(57) ABSTRACT

The present disclosure provides compositions and methods for treating an autoimmune disorder or cancer in a subject. In some embodiments, the methods include the use of modified T cells (e.g., CD8⁺ T cells) that have high layilin expression. In other embodiments, the methods include the use of layilin-binding proteins. Also provided herein are methods and compositions for identifying modulators of layilin or beta-integrin complex interaction.

### CROSS REFERENCE TO RELATED APPLICATIONS

This application is a continuation of PCT/US2020/017557, which claims the benefit of U.S. Provisional Application Nos. 62/802,855 filed on Feb. 8, 2019 and 62/880,022 filed on Jul. 29, 2019, each of which is hereby incorporated in its entirety by reference for all purposes.

### STATEMENT AS TO RIGHTS TO INVENTIONS MADE UNDER FEDERALLY SPONSORED RESEARCH AND DEVELOPMENT

This invention was made with government support under grant no. R21 AR072195 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 6, 2020, is named 081906-1177114-235320WO_SL.txt and is 44,873 bytes in size.

### BACKGROUND

Autoimmunity results from a dysfunction of the immune system. The immune system produces auto-antibodies that attack healthy cells, tissues and/or organs. Autoimmune diseases can affect any part of the body and more than 80 autoimmune diseases have been identified, including Type-1 diabetes, rheumatoid arthritis, and multiple sclerosis. Autoimmunity is characterized by the reaction of cells or proteins (e.g., auto-antibodies) of the immune system against the organism's own antigens (e.g., auto-antigens). Autoimmunity may be part of the organism's own physiological immune response (e.g., natural autoimmunity) or may be pathologically induced. Different mechanisms (which may not be mutually exclusive) involved in the induction and progression of a pathological autoimmunity include, for example, genetic or acquired defects in immune tolerance or immune regulatory pathways, molecular mimicry to viral or bacterial protein, and/or an impaired clearance of apoptotic cell materials.

Cancer is the second leading cause of morbidity, accounting for nearly 1 in 6 of all deaths globally. Of the 8.8 million deaths caused by cancer in 2015, the cancers that claimed the most lives were from lung cancer (1.69 million), liver cancer (788,000), colorectal cancer (774,000), stomach cancer (754,000), and breast cancer (571,000). The economic impact of cancer in 2010 was estimated to be USD1.16 Trillion, and the number of new cases is expected to rise by approximately 70% over the next two decades (World Health Organization Cancer Facts 2017).

Layilin is a protein encoded by the LAYN gene on chromosome 11 in the human genome. Hyaluronic acid is the only presently known ligand of layilin. Antagonists of the interaction of layilin with hyaluronic acid such as hyaluronan oligomers may be used for the treatment of multi-drug resistant cells (see, e.g., US Patent Publication No. US20040229843). It has also been reported that layilin is upregulated in CD8⁺ T cells in patients with liver cancer (see, e.g., Zheng et al., Cell 169:1342-1356, 2017).

### SUMMARY

In one aspect, the disclosure features a method for treating an autoimmune disorder in a subject in need thereof, comprising administering to the subject a therapeutically effective amount of a layilin-binding protein which inhibits the activity of layilin. In some embodiments of this aspect, the autoimmune disorder has a pathogenicity associated with the presence of CD8⁺ T cells in a diseased tissue.

In some embodiments, the layilin-binding protein which inhibits the activity of layilin is an anti-layilin antibody or a fragment thereof. The anti-layilin antibody may be a full-length antibody, a Fab, a F(ab′)2, an Fv, a single chain Fv (scFv) antibody, a V_{H}, or a V_{H}H.

In some embodiments of this aspect, the layilin-binding protein which inhibits the activity of layilin binds to an epitope on a domain of layilin that binds to its natural ligand(s) e.g. hyaluronic acid. In some embodiments of this aspect, the layilin-binding protein which inhibits the activity of layilin prevents or inhibits the binding of layilin to its natural ligand(s) e.g. hyaluronic acid. In some embodiments, the layilin-binding protein which inhibits the activity of layilin interferes with the binding of a beta integrin complex expressed on CD8+ T cells to cell adhesion molecules and/or inhibits beta integrin complex activation.

In certain embodiments, the anti-layilin antibody which inhibits the activity of layilin is a bispecific antibody. In some embodiments, a first variable domain of the bispecific antibody which inhibits the activity of layilin binds to layilin protein and a second variable domain of the bispecific antibody binds to an antigen expressed on the CD8⁺ T cells.

In some embodiments, the autoimmune disorder is in a tissue. In particular embodiments, the autoimmune disorder is an autoimmune skin disorder (e.g., psoriasis, vitiligo, pemphigus vulgaris, pemphigus foliaceus, bullous pemphigoid, cicatricial pemphigoid, autoimmune alopecia, dermatitis herpetiformis, atopic dermatitis, or chronic autoimmune urticaria).

In some embodiments, the autoimmune disorder is an autoimmune lung disorder (e.g., lung scleroderma).

In some embodiments, the autoimmune disorder is an autoimmune gut disorder (e.g., Crohn's disease, ulcerative colitis, or celiac disease).

In another aspect, the disclosure features a layilin-binding protein for use in the treatment of an autoimmune disorder in a subject. In some embodiments, the autoimmune disorder has a pathogenicity associated with the presence of CD8⁺ T cells in a diseased tissue.

In another aspect, the disclosure features the use of a layilin-binding protein for the manufacture of a medicament for the treatment of an autoimmune disorder in a subject. In some embodiments, the autoimmune disorder has a pathogenicity associated with the presence of CD8⁺ T cells in a diseased tissue.

In another aspect, the disclosure features a method for treating cancer in a subject in need thereof, comprising administering to the subject a modified CD8⁺ T cell having an increased layilin expression relative to an unmodified CD8⁺ T cell. In some embodiments, the modified CD8⁺ T cell is an autologous CD8⁺ T cell. In some embodiments, the modified CD8⁺ T cell is modified ex vivo. In some embodiments, the modified CD8⁺ T cell is a chimeric antigen receptor (CAR) T cell.

In another aspect, the disclosure features a modified CD8⁺ T cell for use in the treatment of cancer in a subject, wherein the modified CD8⁺ T cell has an increased layilin expression relative to an unmodified CD8⁺ T cell. In some embodiments, the modified CD8⁺ T cell is an autologous CD8⁺ T cell. In some embodiments, the modified CD8⁺ T cell is modified ex vivo. In some embodiments, the modified CD8⁺ T cell is a CAR T cell.

In another aspect, the disclosure features the use of a modified CD8⁺ T cell for the manufacture of a medicament for the treatment of cancer in a subject in need thereof, wherein the modified CD8⁺ T cell has an increased layilin expression relative to an unmodified CD8⁺ T cell. In some embodiments, the modified CD8⁺ T cell is an autologous CD8⁺ T cell. In some embodiments, the modified CD8⁺ T cell is modified ex vivo. In some embodiments, the modified CD8⁺ T cell is a CAR T cell.

In another aspect, the disclosure features a method for treating cancer in a subject in need thereof, comprising: (a) modifying ex vivo a CD8⁺ T cell to have an increased layilin expression relative to an unmodified CD8⁺ T cell; (b) optionally expanding the modified CD8⁺ T cell; and (c) introducing the modified CD8⁺ T cell to the subject. In some embodiments of this aspect, the method further comprises, prior to step (a), obtaining a CD8⁺ T cell from the subject to be modified in step (a). In some embodiments, the cancer is a skin cancer (e.g., cutaneous melanoma). In some embodiments, the cancer is a metastatic cancer. In certain embodiments, the modified CD8⁺ T cell is a CAR T cell.

In another aspect, the disclosure features a modified CAR T cell comprising an increased layilin expression relative to an unmodified T cell. In certain embodiments, the modified CAR T cell is CD8⁺. In some embodiments, the modified CAR T cell is derived from an autologous T cell. In certain embodiments, the modified CAR T cell is modified ex vivo.

In another aspect, the disclosure features a method for treating cancer in a subject in need thereof, comprising administering to the subject a modified CART cell having an increased layilin expression relative to an unmodified T cell. In some embodiments, the modified CAR T cell is derived from an autologous T cell. In some embodiments, the modified CAR T cell is modified ex vivo. In some embodiments, the modified CAR T cell is CD8⁺.

In another aspect, the disclosure features a modified CAR T cell for use in the treatment of cancer in a subject, wherein the modified CAR T cell has an increased layilin expression relative to an unmodified T cell. In some embodiments, the modified CAR T cell is derived from an autologous T cell. In some embodiments, the modified CAR T cell is modified ex vivo. In some embodiments, the modified CAR T cell is CD8⁺.

In another aspect, the disclosure features the use of an modified CAR T cell for the manufacture of a medicament for the treatment of cancer in a subject in need thereof, wherein the modified CAR T cell has an increased layilin expression relative to an unmodified T cell. In some embodiments, the modified CAR T cell is derived from an autologous T cell. In some embodiments, the modified CAR T cell is modified ex vivo. In some embodiments, the modified CAR T cell is CD8⁺.

In another aspect, the disclosure features a method for treating cancer in a subject in need thereof, comprising: (a) modifying ex vivo a CAR T cell to have an increased layilin expression relative to an unmodified T cell; (b) optionally expanding the modified CAR T cell; and (c) introducing the modified CAR T cell to the subject. In some embodiments, the method further comprises, prior to step (a), obtaining a CAR T cell to be modified in step (a). In some embodiments, the CAR T cell is derived from an autologous T cell. In some embodiments, the cancer is a skin cancer (e.g., cutaneous melanoma). In some embodiments, the cancer is a metastatic cancer. In some embodiments, the modified CAR T cell is CD8⁺.

In another aspect, the disclosure features a method for treating cancer in a subject in need thereof, comprising administering to the subject a therapeutically effective amount of a layilin-binding protein which enhances the activity of layilin. In another aspect, the disclosure features a layilin-binding protein which enhances the activity of layilin for use in the treatment of cancer in a subject. In another aspect, the disclosure features the use of a layilin-binding protein which enhances the activity of layilin for the manufacture of a medicament for the treatment of cancer in a subject. In some embodiments, the layilin-binding protein which enhances the activity of layilin is an anti-layilin antibody or a fragment thereof. The anti-layilin antibody may be a full-length antibody, a Fab, a F(ab′)2, an Fv, a single chain Fv (scFv) antibody, a V_{H}, or a V_{H}H, especially a full-length antibody. In some embodiments, the layilin-binding protein which enhances the activity of layilin promotes the binding of a beta integrin complex expressed on CD8+ T cells to cell adhesion molecules and/or promotes beta integrin complex activation. In some embodiments, the layilin-binding protein which enhances the activity of layilin promotes the binding of layilin to its natural ligand(s) e.g. hyaluronic acid.

The disclosure also features a method of identifying a modulator of layilin interacting with a layilin interaction partner, comprising: a) providing a layilin protein or a fragment thereof, or a first cell expressing the layilin protein; b) exposing a layilin interaction partner, or a second cell expressing the layilin interaction partner, to the layilin protein or first cell in the presence of a sample, wherein the sample comprises the modulator; c) determining the level of interaction between the layilin protein or first cell to the layilin interaction partner or second cell in the presence of the sample; d) identifying the modulator in the sample as: 1. an inhibitor of layilin interacting with the layilin interaction partner if the level of interaction determined in step (c) is less than the level of interaction determined in the presence of a sample known to not comprise the modulator under otherwise identical conditions, or 2. an activator of layilin interacting with the layilin interaction partner if the level of interaction determined in step (c) is greater than the level of interaction determined in the presence of a sample known to not comprise the modulator under otherwise identical conditions.

In some embodiments, the interaction comprises direct binding between the layilin protein or first cell to the layilin interaction partner or second cell. In some embodiments, the interaction comprises formation of a complex, wherein the complex comprises the layilin protein and the layilin interaction partner. In some embodiments, the layilin protein and the layilin interaction partner comprise human-derived amino acid sequences. In some embodiments, the layilin protein comprises the peptide sequence of any one of SEQ ID NOs. 1-3 or 6-8. In some embodiments, the layilin interaction partner comprises a layilin ligand. In some embodiments, the layilin ligand comprises hyaluronic acid. In some embodiments, the layilin interaction partner comprises a beta integrin complex. In some embodiments, the beta integrin complex comprises a LFA-1 complex or constituents thereof. In some embodiments, the LFA-1 complex constituents comprise integrins beta 2 and alpha L. In some embodiments, the LFA-1 complex comprises an active conformation. In some embodiments, the LFA-1 complex is capable of being bound by an anti-LFA-1 m24 clone. In some embodiments, the layilin interaction partner comprises a beta integrin complex interaction partner. In some embodiments, the beta integrin complex interaction partner comprises talin.

In some embodiments, the modulator is selected from the group consisting of: a binding reagent, an RNAi nucleic acid, a CRISPR system complex, and a small molecule. In some embodiments, the binding reagent comprises an antibody or antigen-binding fragment thereof. In some embodiments, the antibody comprises an anti-layilin antibody or binding fragment thereof. In some embodiments, the antibody comprises an anti-LFA-1 antibody or binding fragment thereof. In some embodiments, the modulator is known or suspected to directly bind to the layilin protein. In some embodiments, the modulator is known or suspected to directly bind to the layilin interaction partner. In some embodiments, the modulator is capable of altering expression of the layilin protein or the layilin interaction partner.

In some embodiments, the sample further comprises a second modulator. In some embodiments, the second modulator is known or suspected to inhibit the activity of the modulator of layilin interacting with the layilin interaction partner. In some embodiments, the modulator of layilin interacting with the layilin interaction partner is known or suspected to directly bind to the layilin protein. In some embodiments, the identifying step (d) identifies the second modulator as an inhibitor of the activity of the modulator of layilin interacting with the layilin interaction partner. In some embodiments, the identifying step (d) identifies the second modulator as an activator of the activity of the modulator of layilin interacting with the layilin interaction partner.

In some embodiments, the sample is selected from the group consisting of: protein, purified protein, lysate, blood, leukapheresis products, supernatant, saliva, urine, tissue, tissue homogenates, stool, and spinal fluid.

In some embodiments, the determining step (c) comprises an assay selected from the group consisting of: a competitive binding assay, a colorimetric assay, an ELISA, a proximity ligation assay, biosensor, flow cytometry, immunohistochemistry, and a cell adhesion assay. In some embodiments, the ELISA comprises a competitive ELISA.

The disclosure also provides a method of identifying modulators of layilin interacting with a layilin interaction partner, comprising: a) providing a layilin protein or a fragment thereof, or a first cell expressing the layilin protein; b) exposing a layilin interaction partner, or a second cell expressing the layilin interaction partner, to the layilin protein or first cell in the presence of a sample, wherein the sample comprises a modulator known to be an activator of layilin interacting with the layilin interaction partner, and wherein the sample is known or suspected to comprise a second modulator; c) determining the level of interaction between the layilin protein or first cell to the layilin interaction partner or second cell in the presence of the sample; d) identifying the sample as: 1. comprising the second modulator, wherein the second modulator is an inhibitor of the modulator of layilin interacting with the layilin interaction partner if the level of interaction determined in step (c) is less than the level of interaction determined in the presence of a sample known to not comprise the second modulator under otherwise identical conditions, 2. comprising the second modulator, wherein the second modulator is an activator of the modulator of layilin interacting with the layilin interaction partner if the level of interaction determined in step (c) is greater than the level of interaction determined in the presence of a sample known to not comprise the second modulator under otherwise identical conditions, or 3. not comprising the second modulator if the level of interaction determined in step (c) is the same, or fails to exceed a threshold considered greater or less than, the level of interaction determined in the presence of a sample known to not comprise the second modulator under otherwise identical conditions.

The disclosure also provides a method of identifying modulators of layilin interacting with a layilin interaction partner, comprising: a) providing a layilin protein or a fragment thereof, or a first cell expressing the layilin protein; b) exposing a layilin interaction partner, or a second cell expressing the layilin interaction partner, to the layilin protein or first cell in the presence of a sample, wherein the sample comprises a modulator known to be an inhibitor of layilin interacting with the layilin interaction partner, and wherein the sample is known or suspected to comprise a second modulator; c) determining the level of interaction between the layilin protein or first cell to the layilin interaction partner or second cell in the presence of the sample; d) identifying the sample as: 1. comprising the second modulator, wherein the second modulator is an inhibitor of the modulator of layilin interacting with the layilin interaction partner if the level of interaction determined in step (c) is greater than the level of interaction determined in the absence of the second binding reagent under otherwise identical conditions, or 2. comprising the second modulator, wherein the second modulator is an activator of the modulator of layilin interacting with the layilin interaction partner if the level of interaction determined in step (c) is less than the level of interaction determined in the absence of the second binding reagent under otherwise identical conditions.

The disclosure also provides a composition for identifying a modulator of layilin interacting with a layilin interaction partner, comprising: a) a layilin protein or a fragment thereof, or a first cell expressing the layilin protein; b) a layilin interaction partner, or a second cell expressing the layilin interaction partner; c) a sample, wherein the sample comprises the modulator, wherein the layilin protein and the layilin interaction partner are configured to interact in the presence of the sample.

In some embodiments, the layilin protein and the layilin interaction partner comprise human-derived amino acid sequences. In some embodiments, the layilin protein comprises the peptide sequence of any one of SEQ ID NOs. 1-3 or 6-8. In some embodiments, the layilin interaction partner comprises a layilin ligand. In some embodiments, the layilin ligand comprises hyaluronic acid. In some embodiments, the layilin interaction partner comprises a beta integrin complex. In some embodiments, the beta integrin complex comprises a LFA-1 complex or constituent thereof. In some embodiments, the LFA-1 complex constituents comprise integrins beta 2 and alpha L. In some embodiments, the LFA-1 complex comprises the peptide sequences shown in SEQ ID NO: 4 and SEQ ID NO: 5. In some embodiments, the LFA-1 complex comprises an active conformation. In some embodiments, the LFA-1 complex is capable of being bound by an anti-LFA-1 m24 clone.

The disclosure also provides a method of identifying a modulator of a beta integrin complex interacting with a beta integrin complex interaction partner, comprising: a) providing a beta integrin complex, a constituent thereof, or a fragment thereof, or a first cell expressing the beta integrin complex, the constituent thereof, or the fragment thereof; b) exposing a beta integrin complex interaction partner, or a second cell expressing the beta integrin complex interaction partner, to the beta integrin complex or first cell in the presence of a sample, wherein the sample comprises the modulator; c) determining the level of interaction between the beta integrin complex or first cell to the beta integrin complex interaction partner or second cell in the presence of the sample; d) the modulator in the sample as: 1. an inhibitor of beta integrin complex interacting with the beta integrin complex interaction partner if the level of interaction determined in step (c) is less than the level of interaction determined in the presence of a sample known to not comprise the modulator under otherwise identical conditions, or 2. an activator of beta integrin complex interacting with the beta integrin complex interaction partner if the level of interaction determined in step (c) is greater than the level of interaction determined in the presence of a sample known to not comprise the modulator under otherwise identical conditions.

The disclosure also provides a method of identifying a modulator of a beta integrin complex interacting with a beta integrin complex interaction partner, comprising: a) providing a beta integrin complex, a constituent thereof, or a fragment thereof, or a first cell expressing the beta integrin complex, the constituent thereof, or the fragment thereof, wherein the beta integrin complex comprises LFA-1; b) exposing a beta integrin complex interaction partner, or a second cell expressing the beta integrin complex interaction partner, to the beta integrin complex or first cell in the presence of a sample, wherein the sample comprises the modulator; c) determining the level of interaction between the beta integrin complex or first cell to the beta integrin complex interaction partner or second cell in the presence of the sample; d) the modulator in the sample as: 1. an inhibitor of beta integrin complex interacting with the beta integrin complex interaction partner if the level of interaction determined in step (c) is less than the level of interaction determined in the presence of a sample known to not comprise the modulator under otherwise identical conditions, or 2. an activator of beta integrin complex interacting with the beta integrin complex interaction partner if the level of interaction determined in step (c) is greater than the level of interaction determined in the presence of a sample known to not comprise the modulator under otherwise identical conditions.

The disclosure also provides a method of identifying a modulator of a beta integrin complex interacting with a beta integrin complex interaction partner, comprising: a) providing a beta integrin complex, a constituent thereof, or a fragment thereof, or a first cell expressing the beta integrin complex, the constituent thereof, or the fragment thereof; b) exposing a beta integrin complex interaction partner, or a second cell expressing the beta integrin complex interaction partner, to the beta integrin complex or first cell in the presence of a sample, wherein the sample comprises the modulator, wherein the modulator is an anti-layilin antibody or antigen-binding fragment thereof; c) determining the level of interaction between the beta integrin complex or first cell to the beta integrin complex interaction partner or second cell in the presence of the sample; d) the modulator in the sample as: 1. an inhibitor of beta integrin complex interacting with the beta integrin complex interaction partner if the level of interaction determined in step (c) is less than the level of interaction determined in the presence of a sample known to not comprise the modulator under otherwise identical conditions, or 2. an activator of beta integrin complex interacting with the beta integrin complex interaction partner if the level of interaction determined in step (c) is greater than the level of interaction determined in the presence of a sample known to not comprise the modulator under otherwise identical conditions.

In some embodiments, the interaction comprises direct binding between the beta integrin complex or first cell to the beta integrin complex interaction partner or second cell. In some embodiments, the interaction comprises formation of a complex, wherein the complex comprises the beta integrin complex and the beta integrin complex interaction partner. In some embodiments, the beta integrin complex and the beta integrin complex interaction partner comprise human-derived amino acid sequences. In some embodiments, the beta integrin complex comprises a LFA-1 complex or constituent thereof. In some embodiments, the LFA-1 complex constituents comprise integrins beta 2 and alpha L. In some embodiments, the LFA-1 complex comprises the peptide sequences shown in SEQ ID NO: 4 and SEQ ID NO: 5. In some embodiments, the LFA-1 complex comprises an active conformation. In some embodiments, the LFA-1 complex is capable of being bound by an anti-LFA-1 m24 clone.

In some embodiments, the modulator is known or suspected to directly bind to the beta integrin complex. In some embodiments, the modulator is known or suspected to directly bind to the beta integrin complex interaction partner. In some embodiments, the modulator is selected from the group consisting of: a binding reagent, an RNAi nucleic acid, a CRISPR system complex, and a small molecule. In some embodiments, the modulator is capable of altering expression of the beta integrin complex or the beta integrin complex interaction partner. In some embodiments, the binding reagent comprises an antibody or antigen-binding fragment thereof. In some embodiments, the antibody comprises an anti-LFA-1 antibody or antigen-binding fragment thereof. In some embodiments, the antibody comprises an anti-layilin antibody or antigen-binding fragment thereof.

In some embodiments, the beta integrin complex interaction partner comprises a ligand. In some embodiments, the ligand comprises ICAM-1. In some embodiments, the beta integrin complex interaction partner comprises an intracellular domain known or suspected to interact with an intracellular domain of the beta integrin complex. In some embodiments, the beta integrin complex interaction partner comprises layilin. In some embodiments, the beta integrin complex interaction partner comprises talin. In some embodiments, the beta integrin complex interaction partner comprises an anti-LFA-1 m24 clone.

In some embodiments, the sample further comprises a second modulator. In some embodiments, the second modulator is known or suspected to inhibit the activity of the modulator of the beta integrin complex interacting with the beta integrin complex interaction partner. In some embodiments, the modulator of the beta integrin complex interacting with the beta integrin complex interaction partner is known or suspected to directly bind to the beta integrin complex interaction partner. In some embodiments, the modulator of the beta integrin complex interacting with the beta integrin complex interaction partner is known or suspected to directly bind to the beta integrin complex. In some embodiments, the identifying step (d) identifies the second modulator as an inhibitor of the activity of the beta integrin complex interacting with the beta integrin complex interaction partner. In some embodiments, the identifying step (d) identifies the second modulator as an activator of the activity of the modulator of the beta integrin complex interacting with the beta integrin complex interaction partner.

In some embodiments, the sample is selected from the group consisting of: protein, purified protein, lysate, blood, leukapheresis products, supernatant, saliva, urine, tissue, tissue homogenates, stool, and spinal fluid.

In some embodiments, the determining step (c) comprises an assay selected from the group consisting of: a competitive binding assay, a colorimetric assay, an ELISA, a proximity ligation assay, biosensor, flow cytometry, immunohistochemistry, and a cell adhesion assay. In some embodiments, the ELISA comprises a competitive ELISA.

The disclosure also provides a method of identifying a modulator of beta integrin complex interacting with a beta integrin complex interaction partner, comprising: a) providing a beta integrin complex, a constituent thereof, or a fragment thereof, or a first cell expressing the beta integrin complex, the constituent thereof, or the fragment thereof; b) exposing a beta integrin complex interaction partner, or a second cell expressing the beta integrin complex interaction partner, to the beta integrin complex or first cell in the presence of a sample, wherein the sample comprises a modulator known to be an activator of the beta integrin complex interacting with the beta integrin complex interaction partner, and wherein the sample is known or suspected to comprise a second modulator; c) determining the level of interaction between the beta integrin complex or first cell to the beta integrin complex interaction partner or second cell in the presence of the sample; d) identifying the sample as: 1. comprising the second modulator, wherein the second modulator is an inhibitor of the modulator of the beta integrin complex interacting with the beta integrin complex interaction partner if the level of interaction determined in step (c) is less than the level of interaction determined in the presence of a sample known to not comprise the second modulator under otherwise identical conditions, 2. comprising the second modulator, wherein the second modulator is an activator of the modulator of the beta integrin complex interacting with the beta integrin complex interaction partner if the level of interaction determined in step (c) is greater than the level of interaction determined in the presence of a sample known to not comprise the second modulator under otherwise identical conditions, or 3. not comprising the second modulator if the level of interaction determined in step (c) is the same, or fails to exceed a threshold considered greater or less than, the level of interaction determined in the presence of a sample known to not comprise the second modulator under otherwise identical conditions.

The disclosure also provides a method of identifying a modulator of beta integrin complex interacting with a beta integrin complex interaction partner, comprising: a) providing a beta integrin complex, a constituent thereof, or a fragment thereof, or a first cell expressing the beta integrin complex, the constituent thereof, or the fragment thereof; b) exposing a beta integrin complex interaction partner, or a second cell expressing the beta integrin complex interaction partner, to the beta integrin complex or first cell in the presence of a sample, wherein the sample comprises a modulator known to be an inhibitor of the beta integrin complex interacting with the beta integrin complex interaction partner, and wherein the sample is known or suspected to comprise a second modulator; c) determining the level of interaction between the beta integrin complex or first cell to the beta integrin complex interaction partner or second cell in the presence of the sample; d) identifying the sample as: 1. comprising the second modulator, wherein the second modulator is an inhibitor of the modulator of the beta integrin complex interacting with the beta integrin complex interaction partner if the level of interaction determined in step (c) is greater than the level of interaction determined in the presence of a sample known to not comprise the second modulator under otherwise identical conditions, 2. comprising the second modulator, wherein the second modulator is an activator of the modulator of the beta integrin complex interacting with the beta integrin complex interaction partner if the level of interaction determined in step (c) is less than the level of interaction determined in the presence of a sample known to not comprise the second modulator under otherwise identical conditions, or 3. not comprising the second modulator if the level of interaction determined in step (c) is the same, or fails to exceed a threshold considered greater or less than, the level of interaction determined in the presence of a sample known to not comprise the second modulator under otherwise identical conditions.

The disclosure also provides a composition identifying a modulator of a beta integrin complex interacting with a beta integrin complex interaction partner, comprising: a) a beta integrin complex, a constituent thereof, or a fragment thereof, or a first cell expressing the beta integrin complex, the constituent thereof, or the fragment thereof; b) a beta integrin complex interaction partner, or a second cell expressing the beta integrin complex interaction partner c) a sample, wherein the sample comprises the modulator; wherein the beta integrin complex and the beta integrin complex interaction partner are configured to interact in the presence of the sample.

In some embodiments, the beta integrin complex and the beta integrin complex interaction partner comprise human-derived amino acid sequences. In some embodiments, the beta integrin complex comprises a LFA-1 complex or constituent thereof. In some embodiments, the LFA-1 complex constituents comprise integrins beta 2 and alpha L. In some embodiments, the LFA-1 complex comprises the peptide sequences shown in SEQ ID NO: 4 and SEQ ID NO: 5. In some embodiments, the LFA-1 complex comprises an active conformation. In some embodiments, the LFA-1 complex is capable of being bound by an anti-LFA-1 m24 clone.

In some embodiments, the modulator is known or suspected to directly bind to the beta integrin complex. In some embodiments, the modulator is known or suspected to directly bind to the beta integrin complex interaction partner. In some embodiments, the modulator is selected from the group consisting of: a binding reagent, an RNAi nucleic acid, a genome editing system, and a small molecule. In some embodiments, the modulator is capable of altering expression of the beta integrin complex or the beta integrin complex interaction partner. In some embodiments, the binding reagent comprises an antibody or antigen-binding fragment thereof. In some embodiments, the antibody comprises an anti-LFA-1 antibody or antigen-binding fragment thereof. In some embodiments, the antibody comprises an anti-layilin antibody or antigen-binding fragment thereof.

In some embodiments, the beta integrin complex interaction partner comprises a ligand. In some embodiments, the ligand comprises ICAM-1. In some embodiments, the beta integrin complex interaction partner comprises an intracellular domain known or suspected to interact with an intracellular domain of the beta integrin complex. In some embodiments, the beta integrin complex interaction partner comprises layilin. In some embodiments, the beta integrin complex interaction partner comprises talin. In some embodiments, the beta integrin complex interaction partner comprises an anti-LFA-1 m24 clone.

In some embodiments, the sample further comprises a second modulator. In some embodiments, the second modulator is known or suspected to inhibit the activity of the modulator of the beta integrin complex interacting with the beta integrin complex interaction partner. In some embodiments, the modulator of the beta integrin complex interacting with the beta integrin complex interaction partner is known or suspected to directly bind to the beta integrin complex interaction partner. In some embodiments, the modulator of the beta integrin complex interacting with the beta integrin complex interaction partner is known or suspected to directly bind to the beta integrin complex.

### BRIEF DESCRIPTION OF THE DRAWINGS

FIG. 1A shows Layilin expression on CD8⁺ T cells enriched from human donor peripheral blood samples and cultured four days in the presence of anti-CD3/CD28 activation. A representative flow cytometry analysis is shown together with a summary quantifying data from donors (Symbol pairs correspond to individual donors).

FIG. 1B shows Layilin expression on CD8⁺ T cells enriched from human donor peripheral blood samples and cultured in the presence of anti-CD3/CD28 activation. Shown are the kinetics (Days 0, 2, 4, 7, and 10) of layilin expression . . . .

FIG. 2 shows that layilin was expressed on the most activated CD8⁺ T cells in lesional skin of psoriasis patients. Top 2 rows of display dimensionally reduced t-SNE (t-distributed stochastic neighbor embedding) plots of layilin protein expression and activation protein (CD25, CTLA-4, PD-1, and HLA-DR) expression on CD8⁺ T cells from lesional skin and non-lesional skin combined from 4 patients. Bottom row shows a representative example of a CyTOF contour plot showing high levels of layilin expression on CD8⁺ T cells in lesional psoriatic skin compared to non-lesional skin from a single patient

FIG. 3A shows that Layilin augments CD8⁺ TIL mediated anti-tumor immunity. Layn^{-/-} or wildtype animals were injected subcutaneously with the MC38 tumor cell line and tumor growth quantified by caliper measurements. Symbols and error bars represent mean and SEM at each time point, n=7 per group. Data is representative of two independent experiments. Statistical significance determined by two-way ANOVA (A and B) or unpaired two-tailed t test (C); *P<0.05, ****P<0.0001.

FIG. 3B shows that Layilin augments CD8⁺ TIL mediated anti-tumor immunity. CD8^{cre}Layn^{f/f} and CD8^{cre}Layn^{wt/wt} mice were injected subcutaneously with B16.F10 or MC38 tumor cell lines. Symbols and error bars represent mean and SEM at each time point, n=6-10 per group. Data is representative of three independent experiments. Statistical significance determined by two-way ANOVA (A and B) or unpaired two-tailed t test (C); *P<0.05, ****P<0.0001.

FIG. 3C shows that Layilin augments CD8⁺ TIL mediated anti-tumor immunity. Representative images and quantification of in vivo luciferin bioluminescence imaging taken of mice bearing MC38-LUC2 tumors. Symbols correspond to individual mice. Statistical significance determined by two-way ANOVA (A and B) or unpaired two-tailed t test (C); *P<0.05, ****P<0.0001.

FIG. 3D shows that Layilin is expressed in mouse models and protects against tumor growth. Schematic depiction of our strategy to generate conditional Layn knockout mice specific to CD8⁺ cells.

FIG. 3E shows that Layilin is expressed in mouse models and protects against tumor growth. CD8⁺ T cell frequencies in CD8^{cre}Layn^{f/f} mice were compared to littermate wild type counterparts across several tissues. Symbols represent individual mice.

FIG. 3F shows that Layilin is expressed in mouse models and protects against tumor growth. Quantitative PCR analysis was performed on CD8⁺TCRβ⁺ T cells isolated by FACS from MC38 tumors or spleens. Each symbol corresponds to an individual mouse. Data is representative of two independent experiments.

FIG. 3G shows that Layilin is expressed in mouse models and protects against tumor growth. CD8⁺TCRβ⁺ T cells isolated by FACS from MC38 tumors and spleens were analyzed by western blot.

FIG. 4A shows a schematic depicting the experiment directed to the accumulation of layilin-expressing CD8⁺ T cells in tissues, specifically a competitive adoptive transfer tumor model to elucidate layilin activity on TILs in vivo.

FIG. 4B shows that layilin expression on CD8⁺ T cells enhanced their accumulation in tissues. Two and three weeks following MC38 engraftment and T cell adoptive transfer into Rag^{-/-} hosts, tumor infiltrating T cells were analyzed by flow cytometry. Data is representative of two independent experiments; paired symbols represent single tumors from individual mice. Statistical significance determined by unpaired two-tailed t test (D-G); *P<0.05, **P<0.01, ***P<0.001.

FIG. 4C shows a comparison of granzyme B, IFNγ, and TNFα expression (left, middle, right panels, respectively) between layilin-deficient and control TILs. Two and three weeks following MC38 engraftment and T cell adoptive transfer into Rag^{-/-} hosts, tumor infiltrating T cells were analyzed by flow cytometry. Data is representative of two independent experiments; paired symbols represent single tumors from individual mice. Statistical significance determined by unpaired two-tailed t test (D-G); *P<0.05, **P<0.01, ***P<0.001.

FIG. 4D shows a comparison of PD-1 expression between layilin-deficient and control TILs. Two and three weeks following MC38 engraftment and T cell adoptive transfer into Rag^{-/-} hosts, tumor infiltrating T cells were analyzed by flow cytometry. Data is representative of two independent experiments; paired symbols represent single tumors from individual mice. Statistical significance determined by unpaired two-tailed t test (D-G); *P<0.05, **P<0.01, ***P<0.001.

FIG. 4E shows a comparison in proliferation between layilin-deficient and control TILs. Two and three weeks following MC38 engraftment and T cell adoptive transfer into Rag^{-/-} hosts, tumor infiltrating T cells were analyzed by flow cytometry. Data is representative of two independent experiments; paired symbols represent single tumors from individual mice. Statistical significance determined by unpaired two-tailed t test (D-G); *P<0.05, **P<0.01, ***P<0.001.

FIG. 4F shows a comparison in the number of granzyme B and IFNγ producing CD8⁺ T cells in tumors between layilin-deficient and control TILs. Two and three weeks following MC38 engraftment and T cell adoptive transfer into Rag^{-/-} hosts, tumor infiltrating T cells were analyzed by flow cytometry. Data is representative of two independent experiments; paired symbols represent single tumors from individual mice. Statistical significance determined by unpaired two-tailed t test (D-G); *P<0.05, **P<0.01, ***P<0.001.

FIG. 4G shows a comparison in the accumulation of CD4 T cells. Three weeks following MC38 engraftment and T cell adoptive transfer into Rag^{-/-} hosts tumor infiltrating T cells were analyzed by flow cytometry. Data is representative of two independent experiments; paired symbols represent single tumors from individual mice. Statistical significance determined by unpaired two-tailed t test (F); *P<0.05.

FIG. 5 shows three exemplary amino acid sequences of layilin (SEQ ID NOS: 1-3).

FIG. 6A shows that layilin enhances LFA-1 activation to promote T cell adhesion. Volcano plot comparing LAYN positive (+) and LAYN negative (−) cells from scRNA-seq analysis (as shown in FIG. 6F) highlighting the top differentially expressed genes between the two populations are shown.

FIG. 6B shows that layilin enhances LFA-1 activation to promote T cell adhesion. Comparison of differentially expressed genes coding for integrin proteins and other adhesion molecules between LAYN positive (+) and negative (−) cells from scRNA-seq analysis are shown.

FIG. 6C shows that layilin enhances LFA-1 activation to promote T cell adhesion. Flow plot of proximity ligation assay (PLA) on activated primary human CD8⁺ T cells are shown. Representative of three experiments.

FIG. 6D shows that layilin enhances LFA-1 activation to promote T cell adhesion. Shown is a static adhesion assay comparing the percentage of LAYN deleted and control primary human CD8⁺ T cells adhering to ICAM-1 coated plates under the following conditions: no stimulation, PMA stimulation, and with addition of an LFA-1-specific blocking antibody. Data is representative of 3 independent experiments; mean and SEM shown.

FIG. 6E shows that layilin enhances LFA-1 activation to promote T cell adhesion. Quantification of flow cytometric plots of the percentage of activated integrin LFA-1 (as detected by clone m24) between control and LAYN overexpressing Jurkat cells under the following conditions are shown: no stimulation, MnCl₂ stimulation, dose-response of addition of an anti-layilin cross-linking antibody (25 μg/ml, 50 μg/ml, 100 μg/ml), and with addition of a isotype (100 μg/ml) control for the layilin antibody. Data is representative of 2 independent experiments and normalized to MnCl₂ positive control; mean and SEM shown. Statistical significance determined by two-way ANOVA; ****P<0.0001.

FIG. 6F shows that layilin enhances LFA-1 activation to promote T cell adhesion. Representative flow cytometric plots of the percentage of activated integrin LFA-1 (as detected by clone m24) between control and LAYN overexpressing Jurkat cells under the following conditions are shown: no stimulation, MnCl₂ stimulation, dose-response of addition of an anti-layilin cross-linking antibody (25 μg/ml, 50 μg/ml, 100 μg/ml), and with addition of a isotype (100 μg/ml) control for the layilin antibody. Data is representative of 2 independent experiments and normalized to MnCl₂ positive control; mean and SEM shown. Statistical significance determined by two-way ANOVA; ****P<0.0001.

FIG. 7A shows that Layilin is highly expressed on CD8⁺PD-1^{hi}CTLA-4^{hi} TILs in human metastatic melanoma. Schematic of the project design and approach for sequencing of CD8⁺PD-1^{hi}CTLA-4^{hi} TILs and layilin's role is shown.

FIG. 7B shows that Layilin is highly expressed on CD8⁺PD-1^{hi}CTLA-4^{hi} TILs in human metastatic melanoma. Heat map from bulk RNA-seq comparing highest differentially expressed genes between sort-purified PD-1^{hi}CTLA-4^{hi} and PD-1^{lo}CTLA-4^{lo} CD8⁺ TILs is shown.

FIG. 7C shows that Layilin is highly expressed on CD8⁺PD-1^{hi}CTLA-4^{hi} TILs in human metastatic melanoma. Quantification of LAYN RNA counts from bulk RNA-seq; n=5 patients is shown. Each symbol represents an individual patient, mean and SEM shown.

FIG. 7D shows that Layilin is highly expressed on CD8⁺PD-1^{hi}CTLA-4^{hi} TILs in human metastatic melanoma. Representative flow cytometric plot and quantification of cell surface layilin protein expression of PD-1^{hi}CTLA-4^{hi} versus PD-1^{lo}CTLA-4^{lo} CD8⁺ TILs from 10 human melanoma samples are shown. Each symbol represents an individual patient, mean and SEM shown.

FIG. 7E shows the flow cytometric gating and sorting strategy for isolation of CD8⁺ TILs (live CD45⁺ CD3⁺ CD8⁺). Shown is a representative flow cytometric plot to quantify CTLA-4 and PD-1 expression on CD8⁺ TILs. Also shown is a sorting strategy demonstrating how an intracellular staining control including CTLA-4 was used to set the PD-1 gate so that >80% of the sorted PD-1^{hi}CTLA-4^{hi} population expressed high levels of both markers.

FIG. 7F shows comparative analysis of human melanoma TIL subsets with gene set enrichment analysis (GSEA). GSEA showing enrichment of exhaustion, tissue-resident memory, and activation and effector function signatures genes within the ranked gene expression of PD-1^{hi}CTLA-4^{hi} compared to PD-1^{lo}CLTA-4^{lo} CD8⁺ TILs from human melanoma (n=5) are shown.

FIG. 8A shows that layilin expression is enriched on highly activated, clonally expanded CD8⁺ TILs. Feature plots of single-cell RNA-seq (scRNA-seq); n=20,018 cells from four human melanoma samples are shown.

FIG. 8B shows that layilin expression is enriched on highly activated, clonally expanded CD8⁺ TILs. Heat maps comparing selected differentially expressed genes in LAYN positive (+) and LAYN negative (−) cells from scRNA-seq analysis are shown.

FIG. 8C shows that layilin expression is enriched on highly activated, clonally expanded CD8⁺ TILs. scRNA-seq analysis of LAYN expression in peripheral blood, metastatic lymph nodes (involved LN) and primary tumor from patient K-409 is shown.

FIG. 8D shows scRNA-seq analysis of LAYN expression in matched peripheral blood and metastatic lymph node (involved LN). Data shows scRNA-sequencing of CD8⁺ T cells isolated from patient K-411.

FIG. 8E shows that layilin expression is enriched on highly activated, clonally expanded CD8⁺ TILs. Shown are UMAP plots generated from single cell RNA and TCR sequencing demonstrating LAYN expression and clone size from K-409 involved lymph node. Clones are defined as sets of cells with perfect matches for all called TCR α and β chains from single cell TCR data (sc-TCR).

FIG. 8F shows that TCR sequencing of human melanoma sample K-409 primary tumor sample demonstrates that LAYN is associated with clonal expansion. Shown are UMAP plots generated from single cell RNA and TCR sequencing demonstrating LAYN expression and clone size from K-409 primary tumor. Clones are defined as sets of cells with perfect matches for all called TCR α and β chains from single cell TCR data (sc-TCR).

FIG. 8G shows that layilin expression is enriched on highly activated, clonally expanded CD8⁺ TILs. Shown are coxcomb plots showing the 20 most expanded LAYN⁺ and LAYN⁻ clones in K-409 involved lymph node. Each pie slice represents a unique CD8⁺ T cell clonotype, and pie slice height is proportional to clone size. FIG. 8G discloses SEQ ID NOS 13-20, 19, 21-45, 41, 31 and 27, respectively, in order of appearance.

FIG. 8H shows that layilin expression is enriched on highly activated, clonally expanded CD8⁺ TILs. Shown are coxcomb plots showing the 20 most expanded LAYN⁺ and LAYN⁻ clones in K-409 primary tumor. Each pie slice represents a unique CD8⁺ T cell clonotype, and pie slice height is proportional to clone size. FIG. 8H discloses SEQ ID NOS 13-20, 19, 21-23, 46, 14, 24-27, 30-31, 34-37, 47, 35, 40-45, 48, 37, 18, 31 and 25, respectively, in order of appearance.

FIG. 8I shows that layilin expression is enriched on highly activated, clonally expanded CD8⁺ TILs. Shown are representative flow cytometric plot and quantification of cell surface layilin and CD39 protein expression of CD8⁺ TILs from 8 human melanoma samples. Each symbol represents an individual patient, mean and SEM shown.

FIG. 9A shows that Layilin enhances human CD8⁺ T cell cytotoxicity without affecting cellular proliferation, cytokine production or inhibitory receptor expression. Top panel presents the schematic outlining the strategy for CRISPR-Cas9 electroporation-mediated LAYN deletion and introduction of the 1G4 TCR to human CD8⁺ T cells. Representative flow cytometric plot of layilin protein expression between LAYN guide treated and non-targeted guide (Control) is shown. Bottom panels show efficiency of CRISPR/CAS9 deletion of LAYN as quantified by flow cytometry.

FIG. 9B shows that Layilin enhances human CD8⁺ T cell cytotoxicity without affecting cellular proliferation, cytokine production or inhibitory receptor expression. Shown are quantification and representative images of A375 growth and clearance when co-cultured with CRISPR control or LAYN deleted 1G4⁺ T cells. Data is a composite from two donors and representative of three independent experiments; mean and SEM shown.

FIG. 9C shows that Layilin enhances human CD8⁺ T cell cytotoxicity without affecting cellular proliferation, cytokine production or inhibitory receptor expression. Shown is quantification of A375 growth and clearance when co-cultured with CRISPR control or LAYN deleted 1G4⁺ T cells. Data is a composite from two donors and representative of three independent experiments; mean and SEM shown

FIG. 9D shows that Layilin enhances human CD8⁺ T cell cytotoxicity without affecting cellular proliferation, cytokine production or inhibitory receptor expression. A375 melanoma-T cell co-culture supernatants were collected on day five and measured for IFNγ and TNFα secretion by multiplex ELISA is shown. Data is representative of two independent experiments; mean and SD shown.

FIG. 9E shows that Layilin enhances human CD8⁺ T cell cytotoxicity without affecting cellular proliferation, cytokine production or inhibitory receptor expression. A375 melanoma-T cell co-culture supernatants were collected on day five and measured for IFNγ and TNFα secretion by multiplex ELISA. Data is representative of two independent experiments; mean and SD shown. Human CD8⁺ T cells activated with anti-CD3/CD28 were electroporated with Cas9 preloaded with control or LAYN targeting guide RNA, cultured for four days, and analyzed by flow cytometry. Shown is surface receptor expression. Data is representative of three experiments; mean and SD shown for (D). Statistical significance determined by two-way ANOVA, *P<0.05.

FIG. 9F shows that Layilin enhances human CD8⁺ T cell cytotoxicity without affecting cellular proliferation, cytokine production or inhibitory receptor expression. A375 melanoma-T cell co-culture supernatants were collected on day five and measured for IFNγ and TNFα secretion by multiplex ELISA. Data is representative of two independent experiments; mean and SD shown. Human CD8⁺ T cells activated with anti-CD3/CD28 were electroporated with Cas9 preloaded with control or LAYN targeting guide RNA, cultured for four days, and analyzed by flow cytometry. Shown is proliferation. Data is representative of three experiments; mean and SD shown for (D). Statistical significance determined by two-way ANOVA, *P<0.05.

FIG. 9G shows that Layilin enhances human CD8⁺ T cell cytotoxicity without affecting cellular proliferation, cytokine production or inhibitory receptor expression. A375 melanoma-T cell co-culture supernatants were collected on day five and measured for IFNγ and TNFα secretion by multiplex ELISA. Data is representative of two independent experiments; mean and SD shown. Human CD8⁺ T cells activated with anti-CD3/CD28 were electroporated with Cas9 preloaded with control or LAYN targeting guide RNA, cultured for four days, and analyzed by flow cytometry. Shown is intracellular granzyme B. Data is representative of three experiments; mean and SD shown for (D). Statistical significance determined by two-way ANOVA, *P<0.05.

FIG. 9H shows that Layilin enhances human CD8⁺ T cell cytotoxicity without affecting cellular proliferation, cytokine production or inhibitory receptor expression. A375 melanoma-T cell co-culture supernatants were collected on day five and measured for IFNγ and TNFα secretion by multiplex ELISA. Data is representative of two independent experiments; mean and SD shown. Human CD8⁺ T cells activated with anti-CD3/CD28 were electroporated with Cas9 preloaded with control or LAYN targeting guide RNA, cultured for four days, and analyzed by flow cytometry. Shown is IFNγ and TNFα secretion. Data is representative of three experiments; mean and SD shown for (D). Statistical significance determined by two-way ANOVA, *P<0.05.

FIG. 10 shows percentage of CD8 T cells expressing granzyme-B in LAYN⁺ and LAYN⁻ CD8 T cells from skin explants treated with the anti-layilin antibody (clone 3F7D7E2).

FIG. 11A shows layilin is preferentially and highly expressed on a subset of activated Tregs in healthy and diseased human skin. RNA-Seq of Tregs and Teff cells FACS-purified from normal human skin. Tregs and Teffs were sorted purified based on CD25 and CD27 expression. A representative flow plot is shown (left panel). Cells were pre-gated on live CD45⁺ CD3⁺ CD4⁺ CD8⁻ cells. Volcano plot comparing expression profile of Tregs versus Teffs is shown (right panel).

FIG. 11B shows layilin is preferentially and highly expressed on a subset of activated Tregs in healthy and diseased human skin. Shown is RNA-Seq of Tregs and Teff cells FACS-purified from normal human skin. Expression of specific genes identified by RNA-Seq is shown, including layilin, Foxp3, CD27, CTLA-4, CD25 and CD3ε, by skin Tregs relative to skin Teffs.

FIG. 11C shows layilin is preferentially and highly expressed on a subset of activated Tregs in healthy and diseased human skin. RNA-S eq of Tregs and Teff cells FACS-purified from normal human skin. Shown is gene counts of layilin transcripts on Teff and Tregs are shown a. n=5 healthy donors.

FIG. 11D shows layilin expression on Tregs, CD4⁺ Teffs, CD8⁺ T cells, dendritic cells (DC) and keratinocytes (KC), sort-purified from normal human skin, as determined by RNA-Seq. n=7 normal healthy donors. ANOVA used for analysis.

FIG. 11E shows flow cytometric analysis of percentage of layilin⁺ cells within CD4⁺Foxp3⁺ Tregs and CD4⁺Foxp3⁻ Teff populations in human skin versus peripheral blood. n=5-12 healthy donors/group.

FIG. 11F shows flow cytometric analysis of median fluorescence intensity (MFI) of CD25, Foxp3, CTLA4, ICOS and CD27 expression on Layn^{high} Tregs, Layn^{low} Tregs, and CD4⁺ Teff in human skin. n=4 healthy donors. Representative flow plots and their quantification for Tregs is shown.

FIG. 11G shows RNA-Seq analysis of Tregs and Teffs FACS-purified from metastatic tumors of melanoma patients. n=12 melanoma patients. A representative flow plot is shown. Cells were pre-gated on live CD45⁺ CD3⁺ CD4⁺ CD8⁻ cells. Volcano plot comparing expression profile of Tregs versus Teffs is shown.

FIG. 11H shows RNA-Seq analysis of Tregs and Teffs FACS-purified from metastatic tumors of melanoma patients. n=12 melanoma patients. Expression of specific genes identified by RNA-Seq is shown, including layilin, Foxp3, CD27, CTLA-4, CD25 and CD3E.

FIG. 11I shows RNA-Seq analysis of Tregs and Teffs FACS-purified from metastatic tumors of melanoma patients. n=12 melanoma patients. Gene counts of layilin transcripts on Teff and Tregs are shown.

FIG. 11J shows RNA-Seq analysis of Tregs and Teffs FACS-purified from lesional skin of psoriasis patients. n=4-5 psoriasis patients. A representative flow plot is shown (left panel). Cells were pre-gated on live CD45⁺ CD3⁺ CD4⁺ CD8⁻ cells. Volcano plot comparing expression profile of Tregs versus Teffs is shown (right panel).

FIG. 11K shows RNA-Seq analysis of Tregs and Teffs FACS-purified from lesional skin of psoriasis patients. n=4-5 psoriasis patients. Expression of specific genes identified by RNA-Seq is shown, including layilin, Foxp3, CD27, CTLA-4, CD25 and CD3E.

FIG. 11L shows RNA-Seq analysis of Tregs and Teffs FACS-purified from lesional skin of psoriasis patients. n=4-5 psoriasis patients. Gene counts of layilin transcripts on Teff and Tregs are shown.

FIG. 11M shows Uniform Manifold Approximation and Projection (UMAP) embeddings of mass cytometric data with indicated scaled marker intensities. Gated CD4+ T cells (n=11,465 cells) were proportionally sampled from 4 lesional psoriasis skin punch biopsies (top). Paired median signal intensities (MSI) of CD25, FOXP3, CTLA4, and CD27 on LAYN+ and LAYN− Tregs (bottom).

FIG. 12A shows Tregs expressing Layilin have attenuated suppression and activation in vitro. Shown is the experimental scheme of an in vitro Treg suppression assay. CTV-stained Teffs were cocultured with varying proportions of sorted Tregs retrovirally transduced with either Layn-eGFP-pMIG vector (mLayn-Treg) or empty pMIG vector (EV-Treg), in the presence of mitomycin C-treated APCs and 0.5 ug/ml a-CD3 on a fibroblast-coated plate for 72 hours.

FIG. 12B shows Tregs expressing Layilin have attenuated suppression and activation in vitro. Representative histograms and quantification of Teff proliferation, as measured by percentage of undivided Teffs and proliferating Teffs (% of Ki67⁺ Teffs). n=3 replicates/condition. Data representative of 4 independent experiments. Two-way ANOVA with Bonferroni's test for multiple comparisons.

FIG. 12C shows Tregs expressing Layilin have attenuated suppression and activation in vitro. Shown is an in vitro Treg activation assay. Flow cytometric analysis of MFI of CD25, ICOS, LAG3 and FOXP3 expression on mLayn-Tregs compared to EV-Tregs, stimulated for 72 hours, in the presence of APCs and 0.5 ug/ml a-CD3. n=2-3 replicates/condition. Data representative of 5 independent experiments. Unpaired Student's t-test.

FIG. 13A shows that layilin attenuates Treg suppressive capacity in vivo. Foxp3^{Cre}Layn^{fl/fl} or control Foxp3^{Cre} mice were injected s.c. with the MC38 tumor cell line and tumor growth quantified by caliper measurements over time. n=7-8 mice/group. Data representative of 4 independent experiments. Two-way ANOVA with Bonferroni's test for multiple comparisons.

FIG. 13B shows that layilin attenuates Treg suppressive capacity in vivo. Flow cytometric analysis of specific leukocyte populations is shown: IFNγ⁺ and Ki67⁺ CD8⁺ T cells 24 days after MC38 tumor engraftment. Representative flow plots and their quantification is shown. n=7-8 mice/group. Data representative of 3 independent experiments. Unpaired Student's t-test.

FIG. 13C shows that layilin attenuates Treg suppressive capacity in vivo. Flow cytometric analysis of specific leukocyte populations is shown: IFNγ⁺ and Ki67⁺ CD4⁺ Teff cells 24 days after MC38 tumor engraftment. Representative flow plots and their quantification is shown. n=7-8 mice/group. Data representative of 3 independent experiments. Unpaired Student's t-test.

FIG. 13D shows that layilin attenuates Treg suppressive capacity in vivo. Flow cytometric analysis of specific leukocyte populations is shown: total, Ly6C⁺, and CD206⁺ CD11c⁻ macrophages, infiltrating tumors of Foxp3^{Cre}Layn^{fl/fl} or control Foxp3^{Cre} mice, 24 days after MC38 tumor engraftment. Representative flow plots and their quantification is shown. n=7-8 mice/group. Data representative of 3 independent experiments. Unpaired Student's t-test.

FIG. 14A shows layilin expression on Tregs promotes their accumulation in tissues. Shown is flow cytometric quantification of live CD4⁺ CD25⁺Foxp3⁺ Tregs in tumor, tumor draining lymph nodes (DLN) and skin of Foxp3^{Cre}Layn^{fl/fl} mice compared to Foxp3^{Cre} control mice injected s.c. with MC38 cells, represented as percentages and absolute number of cells. Data representative of 3 independent experiments. n=6-7 mice/group. Unpaired Student's t-test.

FIG. 14B shows layilin expression on Tregs promotes their accumulation in tissues using adoptive transfer of Layn-overexpressing Tregs into Foxp3^{DTR} mice. Shown is the experimental scheme. Tregs sorted from CD45.1 mice were expanded ex vivo and retrovirally transduced with either Layn-eGFP-pMIG vector or empty pMIG vector. These cells were i. v. injected into 6-10 weeks old CD45.2 Foxp3^{DTR} mice and host Tregs depleted through administration of DT.

FIG. 14C shows layilin expression on Tregs promotes their accumulation in tissues using adoptive transfer of Layn-overexpressing Tregs into Foxp3^{DTR} mice. Shown is flow cytometric quantification of total CD45.1⁺ CD4⁺ CD25⁺Foxp3⁺ Tregs in skin of CD45.2 Foxp3^{DTR} mice, represented as percentages and absolute number of cells. Data representative of 3 independent experiments. n=3-5 mice/group. Unpaired Student's t-test.

FIG. 14D shows layilin expression on Tregs promotes their accumulation in tissues using adoptive transfer of Layn-overexpressing Tregs into Foxp3^{DTR} mice. Shown is flow cytometric quantification of GFP⁺ CD45.1⁺ Tregs in skin of CD45.2 Foxp3^{DTR} mice, represented as percentages and absolute number of cells. Data representative of 3 independent experiments. n=3-5 mice/group. Unpaired Student's t-test.

FIG. 14E shows layilin expression on Tregs promotes their accumulation in tissues using adoptive transfer of Layn-overexpressing Tregs into Foxp3^{DTR} mice. Shown is flow cytometric quantification of expression of Ki67 on CD45.1⁺ Tregs in skin of CD45.2 Foxp3^{DTR} mice. Data representative of 3 independent experiments. n=3-5 mice/group. Unpaired Student's t-test.

FIG. 15A shows layilin functions to ‘anchor’ Tregs in mouse skin. Shown is intravital two-photon imaging of Tregs in skin of Layn^{--/--} Foxp3GFP mice compared to WT Foxp3GFP mice at steady state, over a period of 60 minutes with xy plots of cell tracks shown.

FIG. 15B shows layilin functions to ‘anchor’ Tregs in mouse skin. Shown is intravital two-photon imaging of Tregs in skin of Layn^{--/--} Foxp3GFP mice compared to WT Foxp3GFP mice at steady state, over a period of 60 minutes with track displacement length shown.

FIG. 15C shows layilin functions to ‘anchor’ Tregs in mouse skin. Shown is intravital two-photon imaging of Tregs in skin of Layn^{--/--} Foxp3^{GFP} mice compared to WT Foxp3^{GFP} mice at steady state, over a period of 60 minutes with track speed means of the tracks shown.

FIG. 15D shows layilin functions to ‘anchor’ Tregs in mouse skin. Shown is intravital two-photon imaging of Tregs in skin of Layn^{--/--} Foxp3^{GFP} mice compared to WT Foxp3^{GFP} mice at steady state, over a period of 60 minutes with sphericity of cells over time shown.

FIG. 15E shows layilin functions to ‘anchor’ Tregs in mouse skin. Shown is intravital two-photon imaging of Tregs in skin of Layn^{--/--} Foxp3^{GFP} mice compared to WT Foxp3^{GFP} mice at steady state, over a period of 60 minutes with mean sphericity of each cell shown.

FIG. 15F shows layilin functions to ‘anchor’ Tregs in mouse skin. Shown is intravital two-photon imaging of Tregs in skin of RAG2^{--/--} mice 6 weeks after being adoptively transferred with Tregs from either Layn^{--/--} Foxp3^{GFP} mice or WT Foxp3^{GFP} mice, over a period of 60 minutes. Shown is the experimental scheme of adoptive transfer of cells.

FIG. 15G shows layilin functions to ‘anchor’ Tregs in mouse skin. Shown is intravital two-photon imaging of Tregs in skin of RAG2^{--/--} mice 6 weeks after being adoptively transferred with Tregs from either Layn^{--/--} Foxp3^{GFP} mice or WT Foxp3^{GFP} mice, over a period of 60 minutes. Shown is xy plots of cell tracks. n=at least 100 cells/group. Data representative of 2-3 independent experiments. Unpaired Student's t-test.

FIG. 15H shows layilin functions to ‘anchor’ Tregs in mouse skin. Shown is intravital two-photon imaging of Tregs in skin of RAG2^{--/--} mice 6 weeks after being adoptively transferred with Tregs from either Layn^{--/--} Foxp3^{GFP} mice or WT Foxp3^{GFP} mice, over a period of 60 minutes. Shown is track displacement length. n=at least 100 cells/group. Data representative of 2-3 independent experiments. Unpaired Student's t-test.

FIG. 15I shows layilin functions to ‘anchor’ Tregs in mouse skin. Shown is intravital two-photon imaging of Tregs in skin of RAG2^{--/--} mice 6 weeks after being adoptively transferred with Tregs from either Layn^{--/--} Foxp3^{GFP} mice or WT Foxp3^{GFP} mice, over a period of 60 minutes. Shown is track speed means of the tracks. n=at least 100 cells/group. Data representative of 2-3 independent experiments. Unpaired Student's t-test.

FIG. 16 shows that layilin is expressed on a subset of activated Tregs in human metastatic melanoma. Shown is a flow cytometric analysis of median fluorescence intensity (MFI) of CD25, FOXP3, CTLA4, and ICOS expression on Layn^{high} Tregs, Layn^{low} Tregs, and CD4⁺ Teff in melanoma samples. n=11 melanoma patients. Representative flow plots and their quantification for Tregs is shown.

FIG. 17A shows layilin mRNA expression on mouse Tregs and in vitro assays. Shown is mRNA expression of Layn relative to HPRT in Tregs and Teffs sort-purified from mouse skin and sdLN. n=3 mice.

FIG. 17B shows layilin mRNA expression on mouse Tregs and in vitro assays. Shown is retroviral transduction efficiency of sorted mouse Tregs, transduced with Layn-eGFP or empty vector-eGFP, as measured by % of GFP⁺ cells, compared to untransduced Tregs directly before adoptive transfer. Representative flow plots are shown. n=3 replicates/condition. Data representative of 4 independent experiments. Two-way ANOVA with Bonferroni's test for multiple comparisons.

FIG. 17C shows layilin mRNA expression on mouse Tregs and in vitro assays. Shown is mRNA expression of mouse Layn relative to HPRT in Tregs transduced with Layn-eGFP vector and compared to untransduced Tregs and a water only control, as measured by qPCR. Data representative of 2-3 independent experiments. n=3 replicates/condition. Data representative of 4 independent experiments. Two-way ANOVA with Bonferroni's test for multiple comparisons.

FIG. 17D shows layilin mRNA expression on mouse Tregs and in vitro assays. Shown is in vitro suppression assay. Suppression of Teff proliferation by Tregs, as measured by division index of proliferating Teffs, in presence of Tregs. n=3 replicates/condition. Data representative of 4 independent experiments. Two-way ANOVA with Bonferroni's test for multiple comparisons.

FIG. 18A shows generation and characterization of Layn^{fl/fl} mice at baseline and in MC38 tumors. Shown is a schematic representation of our strategy to generate conditional Layn knockout mice specific to Tregs.

FIG. 18B shows generation and characterization of Layn^{fl/fl} mice at baseline and in MC38 tumors. Shown is steady state characterization of Layn^{fl/fl} Foxp3^{ERT2Cre} mice injected tamoxifen to specifically knockout layn expression on Tregs mouse compared to control mice injected with corn oil (vehicle) only. Shown is quantification of total live CD45⁺ cells in skin and sdLN of mice by flow cytometry.

FIG. 18C shows generation and characterization of Layn^{fl/fl} mice at baseline and in MC38 tumors. Shown is steady state characterization of Layn^{fl/fl} Foxp3^{ERT2Cre} mice injected tamoxifen to specifically knockout layn expression on Tregs mouse compared to control mice injected with corn oil (vehicle) only. Shown is quantification of CD4⁺ CD25⁺Foxp3⁺ Tregs in skin of mice. Both percentages and absolute numbers of Tregs/gram of skin are shown.

FIG. 18D shows generation and characterization of Layn^{fl/fl} mice at baseline and in MC38 tumors. Shown is steady state characterization of Layn^{fl/fl} Foxp3^{ERT2Cre} mice injected tamoxifen to specifically knockout layn expression on Tregs mouse compared to control mice injected with corn oil (vehicle) only. Shown is MFI expression of CD25, ICOS and CTLA4 on Tregs from skin of mice. n=3-5 mice/group. Data representative of 2 independent experiments.

FIG. 18E shows generation and characterization of Layn^{fl/fl} mice at baseline and in MC38 tumors. Shown is Foxp3^{ERT2-Cre}Layn^{fl/fl} and control Foxp3^{ERT2-Cre} mice both treated with tamoxifen were injected s.c. with the MC38 tumor cell line and tumor growth quantified by caliper measurements over time. n=7-8 mice/group. Data representative of 2 independent experiments. Two-way ANOVA with Bonferroni's test for multiple comparisons.

FIG. 18F shows generation and characterization of Layn^{fl/fl} mice at baseline and in MC38 tumors. Shown is quantification of number of leukocytes infiltrating tumors of Foxp3^{Cre}Layn^{fl/fl} or control Foxp3^{Cre} mice, 24 days after MC38 tumor engraftment. Shown is IFNγ⁺ and Ki67⁺ CD8⁺ T cells. n=5-8 mice/group. Data representative of 3 independent experiments. Unpaired Student's t-test.

FIG. 18G shows generation and characterization of Layn^{fl/fl} mice at baseline and in MC38 tumors. Shown is quantification of number of leukocytes infiltrating tumors of Foxp3^{Cre}Layn^{fl/fl} or control Foxp3^{Cre} mice, 24 days after MC38 tumor engraftment. Shown is IFNγ⁺ and Ki67⁺ CD4⁺ Teff cells. n=5-8 mice/group. Data representative of 3 independent experiments. Unpaired Student's t-test.

FIG. 18H shows generation and characterization of Layn^{fl/fl} mice at baseline and in MC38 tumors. Shown is quantification of number of leukocytes infiltrating tumors of Foxp3^{Cre}Layn^{fl/fl} or control Foxp3^{Cre} mice, 24 days after MC38 tumor engraftment. Shown is total, Ly6C⁺, and CD206⁺ CD11c⁻ macrophages. n=58 mice/group. Data representative of 3 independent experiments. Unpaired Student's t-test.

FIG. 19A shows layilin expression on Tregs promotes their accumulation in tissues. Co-adoptive transfer of Layn-overexpressing (mLayn-Treg) and control empty vector Tregs (EV-Treg) into Foxp3^{DTR} mice. Shown is the experimental scheme. Sorted and ex vivo expanded CD45.1 Tregs were transduced with Layn-eGFP-pMIG and CD45.1.2 Tregs were transduced with empty pMIG vector. Cells were mixed at 1:1 ratio and 3.5×10⁵ total cells were i.v. injected into 6-10 weeks old CD45.2 Foxp3^{DTR} mice. Host Tregs were depleted using DT.

FIG. 19B shows layilin expression on Tregs promotes their accumulation in tissues. Co-adoptive transfer of Layn-overexpressing (mLayn-Treg) and control empty vector Tregs (EV-Treg) into Foxp3^{DTR} mice. Shown is flow cytometric analysis of accumulation of GFP⁺ cells within CD45.1⁺ or CD45.1.2⁺ Treg gate in skin of CD45.2 Foxp3^{DTR} mice, represented as percentages and absolute number of cells. Representative flow plots and their quantification is shown. Data representative of 2 independent experiments. n=4-5 mice/group. Paired Student's t-test

FIG. 19C shows layilin expression on Tregs promotes their accumulation in tissues. Co-adoptive transfer of Layn-overexpressing (mLayn-Treg) and control empty vector Tregs (EV-Treg) into Foxp3^{DTR} mice. Shown is expression of Ki67 on CD45.1⁺ or CD45.1.2⁺ Tregs in skin of CD45.2 Foxp3^{DTR} mice, represented as percentage of cells and MFI of Ki67 expression. Representative flow plots and their quantification is shown. Data representative of 2 independent experiments. n=4-5 mice/group. Paired Student's t-test

FIG. 19D shows layilin expression on Tregs promotes their accumulation in tissues. Co-adoptive transfer of Layn-overexpressing (mLayn-Treg) and control empty vector Tregs (EV-Treg) into Foxp3^{DTR} mice. Shown is flow cytometric analysis of percentage of dead cells, as measured by aqua+ cells, within CD45.1⁺ or CD45.1.2⁺ Treg gate in skin of CD45.2 Foxp3^{DTR} mice. Representative flow plots and their quantification is shown. Data representative of 2 independent experiments. n=4-5 mice/group. Paired Student's t-test.

FIG. 20A shows generation and characterization of Layn^{--/--} mice. Layilin gene consists of 8 exons. Layn^{--/--} mice were created using CRISPR-Cas9 technology by designing single guide RNAs that target exon 1 and exon 4. The sequence targeted within exon 1 (SEQ ID NO: 49) and exon 4 (SEQ ID NO: 50) is shown. Three different founder lines were generated—2 founders had exons 1-4 deleted and one founder had a SNP introduced in exon 4.

FIG. 20B shows generation and characterization of Layn^{--/--} mice. Shown is exon 1-4 deletion confirmed by PCR genotyping using primers specific to the deleted region to compare WT (Layn^{+/+}), Layn^{+/-} and Lay^{-/-} mice. Expected band size in WT mice is ˜210 bp. Two Layn^{-/-} founder mice are shown. SNP mutation was confirmed by qPCR using primers specific to mutation (data not shown).

FIG. 20C shows generation and characterization of Layn^{--/--} mice. Shown is steady state characterization of Layn^{--/--} mouse compared to WT mice. Shown is body weights. n=10-12 mice/group pooled from 4 independent experiments. Similar results were obtained for all 3 founder lines. Results are shown for founder line with SNP mutation, used in FIG. 14.

FIG. 20D shows generation and characterization of Layn^{--/--} mice. Shown is steady state characterization of Layn^{--/--} mouse compared to WT mice. Shown is skin histology by H&E staining.

FIG. 20E shows generation and characterization of Layn^{--/--} mice. Shown is steady state characterization of Layn^{--/--} mouse compared to WT mice. Shown is quantification of total live CD45⁺ cells in skin and sdLN of Layn^{--/--} mice by flow cytometry. n=10-12 mice/group pooled from 4 independent experiments. Similar results were obtained for all 3 founder lines. Results are shown for founder line with SNP mutation, used in FIG. 14.

FIG. 20F shows generation and characterization of Layn^{--/--} mice. Shown is steady state characterization of Layn^{--/--} mouse compared to WT mice. Shown is quantification of CD4⁺ CD25⁺Foxp3⁺ Tregs in skin of Layn^{--/--} mice. Both percentages and absolute numbers of Tregs/gram of skin are shown. n=10-12 mice/group pooled from 4 independent experiments. Similar results were obtained for all 3 founder lines. Results are shown for founder line with SNP mutation, used in FIG. 14.

FIG. 20G shows generation and characterization of Layn^{--/--} mice. Shown is steady state characterization of Layn^{--/--} mouse compared to WT mice. Shown is MFI expression of CD25. CTLA4 and ICOS on Tregs from skin of Layn^{--/--} mice, n=10-12 mice/group pooled from 4 independent experiments. Similar results were obtained for all 3 founder lines. Results are shown for founder line with SNP mutation, used in FIG. 14.

FIG. 21 is an illustration of the active (right) or inactive (left) conformations of LFA-1.

FIG. 22 shows the structure of hyaluronic acid [(C₁₄H₂₁NO₁₁)ₙ)].

### DETAILED DESCRIPTION OF THE EMBODIMENTS

### I. Introduction

The present disclosure provides methods for treating autoimmune disorders and cancer in a subject using proteins that bind layilin or modified cells having high layilin expression, respectively. In methods of treating autoimmune disorders, a layilin-binding protein (e.g., an anti-layilin antibody) may be administered to inhibit or prevent layilin interactions, e.g., inhibiting or preventing the binding of layilin to its natural ligand(s) e.g. hyaluronic acid and/or inhibiting or preventing the binding of a beta integrin complex expressed on CD8+ T cells to cell adhesion molecules and/or inhibiting beta integrin complex activation. In methods of treating cancer, modified T cells (e.g., modified CD8⁺ T cells) having an increased layilin expression relative to unmodified T cells (e.g., unmodified CD8⁺ T cells) may be introduced to a subject. In methods of treating cancer, a layilin-binding protein (e.g., an anti-layilin antibody) may be administered to enhance layilin interactions, e.g., promoting the binding of layilin to its natural ligand(s) e.g. hyaluronic acid and/or promoting the binding of a beta integrin complex expressed on CD8+ T cells to cell adhesion molecules and/or promoting beta integrin complex activation.

### II. Definitions

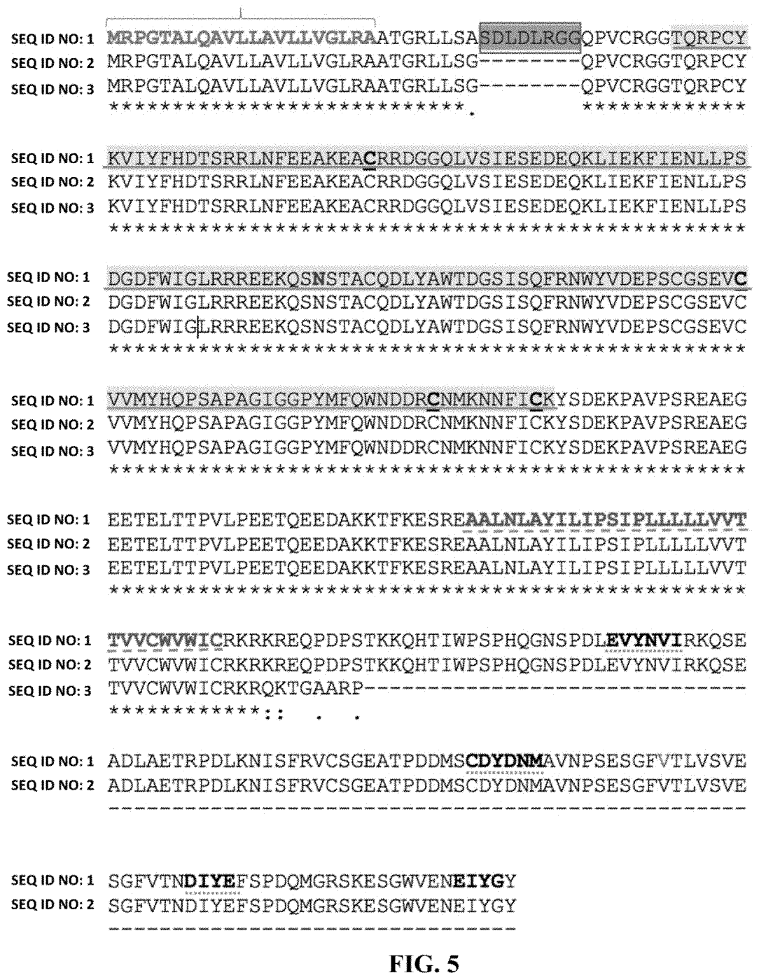

As used herein, the term “layilin” refers a human protein encoded by the LAYN gene on chromosome 11 in the human genome. Layilin can refer to any isoform of layilin including, but not limited to, UniProt Accession numbers Q6UX15-1, Q6UX15-2, Q6UX15-3, herein incorporated by reference for all purposes, with amino acid sequences shown in SEQ ID NOs: 6-8, respectively. Other isoforms include, but are not limited to, UniProt Accession numbers E9PMI0, E9PQU7, A0A0D9SFG0, E9PK64, E9PR90, E9PQY8, herein incorporated by reference for all purposes. Other isoforms include, but are not limited to, Ensembl Accession numbers ENSG00000204381, ENST00000533265, ENST00000533999, ENST00000530962, ENST00000525126, ENST00000525866, ENST00000528924, ENST00000436913, ENST00000375614, herein incorporated by reference for all purposes. Layilin can have the amino acid sequence of any one of SEQ ID NOS: 1-3 (FIG. 5). In some embodiments, layilin has an amino acid sequence that has at least 95% sequence identity (e.g., 97%, 99%, or 100% sequence identity) to the sequence of any one of SEQ ID NOS: 1-3 or 6-8.

As used herein, the term “layilin-binding protein” refers to a molecule that preferentially binds to layilin. In some embodiments, a layilin-binding protein specifically binds to layilin. In some embodiments, a layilin-binding protein may disrupt layilin interactions or cell signaling involving layilin, i.e., inhibit the interaction between layilin and its natural ligand(s) e.g. hyaluronic acid. The structure of hyaluronic acid [(C₁₄H₂₁NO₁₁)ₙ] is shown in FIG. 22. In some embodiments, a layilin-binding protein may interfere with the binding or interaction between layilin and a beta integrin. In some embodiments, by interfering with the binding or interaction between layilin and the beta integrin, the layilin-binding protein can indirectly interfere with the binding of the beta integrin complex expressed on CD8+ T cells to cell adhesion molecules and/or inhibit the beta integrin complex activation. In some embodiments, a layilin-binding protein may promote layilin interactions or cell signaling involving layilin, i.e., promote the interaction between layilin and its natural ligand(s) e.g. hyaluronic acid. In some embodiments, a layilin-binding protein may bind to layilin and stabilize its interaction with a beta integrin. In some embodiments, by binding to layilin and stabilize its interaction with the beta integrin, the layilin-binding protein can also promote the binding of a beta integrin complex expressed on CD8+ T cells to cell adhesion molecules and/or promote beta integrin complex activation. A layilin-binding protein may be an anti-layilin antibody or a fragment thereof. In some embodiments, a layilin-binding protein may alter (e.g., promote or interfere) another protein's interaction with its respective interaction partner. For example, without wishing to be bound by theory, layilin is proposed to form a complex with LFA-1, and the layilin-binding protein may alter LFA-1 interacting with an LFA-1 interaction partner, such as talin or extracellular matrix proteins (e.g., ICAM1).

As used herein, the term “specifically binds” to a target, e.g., layilin, when referring to a layilin-binding protein as described herein, refers to a binding reaction whereby the layilin-binding protein binds to layilin with greater affinity, greater avidity, and/or greater duration than it binds to a different target. In some embodiments, a layilin-binding protein has at least 2-fold, 3-fold, 4-fold, 5-fold, 6-fold, 7-fold, 8-fold, 9-fold, 10-fold, 20-fold, 25-fold, 50-fold, 100-fold, 1,000-fold, 10,000-fold, or greater affinity for layilin compared to an unrelated target when assayed under the same binding affinity assay conditions. The term “specific binding,” “specifically binds to,” or “is specific for” a particular target (e.g., layilin), as used herein, can be exhibited, for example, by a molecule (e.g., a layilin-binding protein) having an equilibrium dissociation constant K_{D} for layilin of, e.g., 10⁻² M or smaller, e.g., 10⁻³ M, 10⁻⁴ M, 10⁻⁵ M, 10⁻⁶ M, 10⁻⁷ M, 10⁻⁸ M, 10⁻⁹M, 10⁻¹⁰ M, 10⁻¹¹ M, or 10⁻¹² M.

As used herein, the term “antibody” herein is used in the broadest sense and encompasses various antibody structures (e.g., full-length or intact antibodies as well as antibody fragments), including but not limited to monoclonal antibodies, polyclonal antibodies, multispecific antibodies (e.g., bispecific antibodies), and antibody fragments. An antibody refers to a polypeptide encoded by an immunoglobulin gene or fragments thereof that specifically binds and recognizes an antigen. Immunoglobulin sequences include the kappa, lambda, alpha, gamma, delta, epsilon, and mu constant region sequences, as well as myriad immunoglobulin variable region sequences. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies include human and other animal antibodies, e.g., mouse and camelid antibodies (including camelid heavy chain only antibodies) and chimeric antibodies (e.g., humanized antibodies). An anti-layilin antibody may be a full-length or intact antibody (i.e. comprises 6 CDRs), or may be a fragment or construct thereof, e.g., a Fab, a F(ab′)₂, an Fv, a single chain Fv (scFv) antibody, a V_{H}, or a V_{H}H.

As used herein, the term “antibody fragments” refers to a portion of a full-length or intact antibody, preferably the antigen binding or variable region of the intact antibody. Examples of antibody fragments include, but are not limited to, a Fab, a F(ab′)₂, an Fv, a single chain Fv (scFv) antibody, a V_{H}, a V_{H}H, and diabodies.

As used herein, the terms “variable region” and “variable domain” refer to the portions of the light and heavy chains of an antibody that include amino acid sequences of complementary determining regions (CDRs, e.g., CDR L1, CDR L2, CDR L3, CDR H1, CDR H2, and CDR H3) and framework regions (FRs). In some embodiments, the amino acid positions assigned to CDRs and FRs are defined according to Kabat (Sequences of Proteins of Immunological Interest, 5th Ed. Public Health Service, National Institutes of Health, Bethesda, Md. (1991)) or EU index of Kabat. Using this numbering system, the actual linear amino acid sequence may contain fewer or additional amino acids corresponding to a shortening of, or insertion into, a CDR or FR of the variable region. The Kabat numbering of residues may be determined for a given antibody by alignment at regions of homology of the sequence of the antibody with a “standard” Kabat numbered sequence.

As used herein, the term “antigen” refers to a polypeptide, glycoprotein, lipoprotein, lipid, carbohydrate, or other agent that is bound (e.g., recognized as “foreign”) by a T cell receptor and/or antibody. Antigens are commonly derived from bacterial, viral, or fungal sources. The term “derived from” may indicate that the antigen is essentially as it exists in its natural antigenic context or that the antigen has been modified to be expressed under certain conditions, i.e., to include only the most immunogenic portion, or to remove other potentially harmful associated components, etc. In the case of an anti-layilin antibody, a layilin protein (e.g., the sequence of any one of SEQ ID NOS: 1-3 or 6-8) or a fragment thereof (e.g., a soluble fragment of layilin; e.g., a domain of layilin that binds to its natural ligand(s) e.g. hyaluronic acid; e.g., a fragment or portion of the sequence of any one of SEQ ID NOS: 1-3 or 6-8) may be used as an antigen.

As used herein, the term “modified T cell” refers to a T cell that has undergone a change (e.g., a genetic change) that causes the modified T cell to exhibit genotypic or phenotypic differences compared to an unmodified T cell. For example, a T cell may be transfected with an expression vector (e.g., a viral vector) containing an expression cassette comprising a nucleic acid encoding a layilin protein to become a modified T cell that has high layilin expression. In another example, a T cell may undergo genomic editing, i.e., by a nuclease, to alter the expression level of the nucleic acid encoding layilin, such that the modified T cell may have a higher or lower expression level of layilin relative to an unmodified T cell. In some embodiments, a modified T cell (e.g., a modified CD8⁺ T cell) may express CD8. In another example, a modified T cell may be a chimeric antigen receptor (CAR) T cell that is derived from an autologous T cell. In some embodiments, the CAR T cell may express CD8.

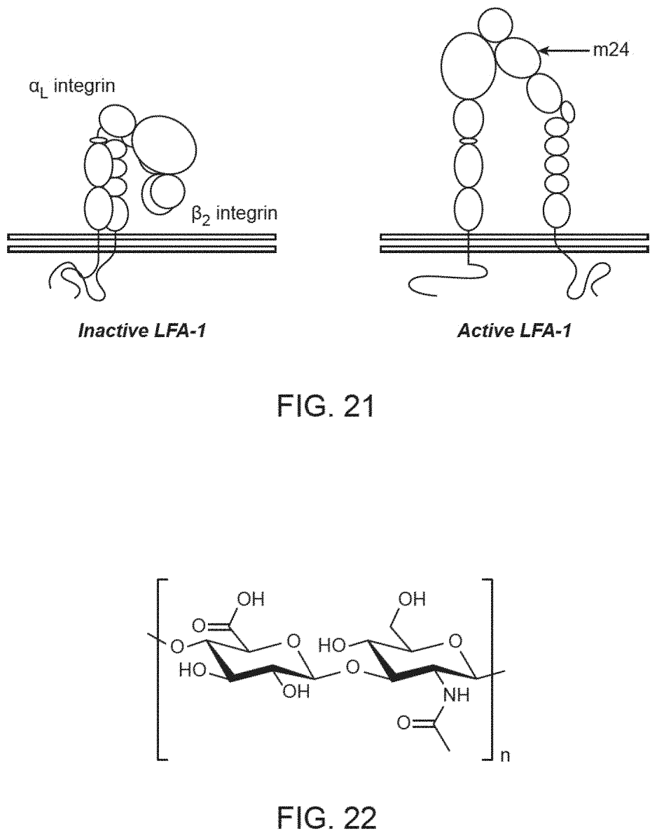

As used herein the term “beta integrin complex” refers to a functional heterodimer complex involving a beta integrin, for example, lymphocyte function-associated antigen 1 (LFA-1). LFA-1 is formed by dimerization of integrins beta 2 and alpha L. LFA-1 is important in immune synapse formation and adhesion of cytotoxic CD8+ T cells during the killing of target cells. Beta integrin complexes can interact with other molecules (also referred to as “beta integrin complex interaction partners”), such other molecules involved in immune synapse formation and/or adhesion. The interaction can be intracellular (e.g., interaction with talin) or extracellular (e.g., an LFA-1 ligand, such as ICAM-1 or other extracellular matrix proteins). The interaction can be directly binding to a partner, such as binding to talin or LFA-1. LFA-1 can interact indirectly with other molecules, such as forming in a complex with other molecules. For example, without wishing to be bound by theory, layilin is proposed to form a complex with (interact indirectly with) LFA-1, where the interaction between layilin and LFA-1 is mediated by both directly binding to talin. LFA-1 can be mammalian LFA-1. LFA-1 can be human LFA-1, such as the complex of human Integrin-Beta 2 (UniProt Accession number P05107, herein incorporated by reference for all purposes), e.g., SEQ ID NO: 4, and human Integrin-Alpha L (UniProt Accession number P20701, herein incorporated by reference for all purposes), e.g., SEQ ID NO: 5. LFA-1 can be in an active or inactive conformation, as illustrated in FIG. 21.

As used herein, the term “unmodified T cell” refers to a wild-type T cell. An unmodified T cell may be one that is isolated from a subject (e.g., a human) having an autoimmune disorder or cancer before the subject has undergone any treatment. In some embodiments, an unmodified T cell may express CD8, e.g., an unmodified CD8⁺ T cell.

As used herein, the term “expression cassette” refers to a nucleic acid construct that, when introduced into a host cell, results in transcription and/or translation of an RNA or polypeptide, respectively. In some embodiments, an expression cassette comprises a promoter operably linked to a polynucleotide encoding a layilin protein. An expression cassette may be placed in an expression vector.

As used herein, the term “subject” refers to a mammal, e.g., preferably a human. Mammals include, but are not limited to, humans and domestic and farm animals, such as monkeys (e.g., a cynomolgus monkey), mice, dogs, cats, horses, and cows, etc.

As used herein, the term “pharmaceutical composition” refers to a medicinal or pharmaceutical formulation that contains an active ingredient as well as one or more excipients and diluents to enable the active ingredient suitable for the method of administration. The pharmaceutical composition may be in aqueous form for intravenous or subcutaneous administration or in tablet or capsule form for oral administration.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present invention, the pharmaceutically acceptable carrier must provide adequate pharmaceutical stability to the active ingredient. The nature of the carrier differs with the mode of administration. For example, for intravenous administration, an aqueous solution carrier is generally used; for oral administration, a solid carrier is preferred.

As used herein, the term “treat” refers to a therapeutic treatment of a disease, e.g., an autoimmune disorder or cancer, in a subject, as well as prophylactic or preventative measures towards the disease. A therapeutic treatment slows the progression of the disease, ameliorates disease symptoms, improves the subject's outcome (e.g., survival), eliminates the disease, and/or reduces or eliminates the symptoms of the disease. Beneficial or desired clinical results include, but are not limited to, alleviation of disease symptoms, diminishment of the extent of the disease, stabilization (i.e., not worsening) of the disease, delay or slowing of the disease progression, amelioration or palliation of the disease state, remission (whether partial or total, whether detectable or undetectable) and prevention of relapse or recurrence of the disease. “Treatment” can also mean prolonging survival as compared to expected survival if not receiving treatment. Those in need of treatment include those already having the disease, condition, or disorder, as well as those at high risk of having the disease, condition, or disorder, and those in whom the disease, condition, or disorder is to be prevented.

### III. T Cells and Layilin

A T cell, or T lymphocyte, is a type of lymphocyte (a subtype of white blood cell) that plays a central role in cell-mediated immunity. T cells can be distinguished from other lymphocytes, such as B cells and natural killer cells, by the presence of a T-cell receptor on the cell surface. A subset of T cells express CD8 glycoprotein on the cell surface, e.g., CD8⁺ T cells. CD8⁺ T cells play a major role in immune responses, such as protection against viral infections and tumors. They perform this function by cytotoxic damage of target cells expressing MHC class I molecules and the relevant antigenic peptide, as well as by the production of effector cytokines such as IFNγ.

Autoreactive CD8⁺ T cells are key players in autoimmune diseases. In particular, CD8⁺ T cells can oppose or promote autoimmune diseases through acting as suppressor cells and as cytotoxic effectors. Studies in several distinct autoimmune models and data from patient samples have established the importance of CD8⁺ T cells in these diseases and defined the mechanisms by which these cells influence autoimmunity. CD8⁺ effectors can promote autoimmune diseases, for example, via dysregulated secretion of inflammatory cytokines, skewed differentiation profiles, and inappropriate induction of apoptosis of target cells. CD8⁺ cells can also protect against autoimmune diseases, for example, by eliminating self-reactive cells and self-antigen sources.

CD8⁺ T cells also play a central role in cancer through their capacity to kill malignant cells upon recognition by T-cell receptor (TCR) of specific antigenic peptides presented on the surface of target cells by human leukocyte antigen class I (HLA-0/beta-2-microglobulin (β2m) complexes. TCR and associated signaling molecules thus are often clustered at the center of the T cell/tumor cell contact area, resulting in formation of an immune synapse (IS) and initiation of a transduction cascade that leads to execution of cytotoxic T lymphocyte (CTL) effector functions. Major CTL activities are mediated either directly, through synaptic exocytosis of cytotoxic granules (e.g., cytotoxic granules containing perforin and granzymes) into the target, resulting in cancer cell destruction, or indirectly, through secretion of cytokines, including interferon (e.g., IFNγ) and tumor necrosis factor (TNF). IFNγ, which is produced by CD8⁺ T cells, can increase the expression of MHC class I antigens by tumor cells, thereby rendering them better targets for CD8⁺ T cells.

Layilin is a cell surface, C-type lectin-like receptor (Borowsky and Hynes, J. Cell Biol. 143:429-442, 1998). Its only currently known ligand is hyaluronic acid (HA) (Bono et al., Exp. Cell Res. 308:177-187, 2001). The intracellular domain of layilin binds to, for example, talin, radixin, and merlin, adaptor molecules that link transmembrane proteins with the actin cytoskeleton (Borowsky and Hynes, supra; Bono et al., supra). Thus, it is thought that layilin plays a role in cell motility and adhesion, linking the extracellular matrix with the cytoskeleton. However, layilin is expressed on both motile and non-motile cells and it is unknown whether it mediates different functions in these different cell types. Accordingly, layilin can interact with other molecules (also referred to as “layilin interaction partners”), such other molecules involved in signaling, motility, and/or adhesion. The interaction can be intracellular (e.g., interaction with talin) or extracellular (e.g., an layilin ligand, such as hyaluronic acid). Layilin can interact directly other molecules, such as talin, a layilin ligand, and/or a layilin-binding protein (e.g., an anti-layilin antibody). Layilin can interact indirectly with other molecules, such as forming in a complex with other molecules. For example, without wishing to be bound by theory, layilin is proposed to form a complex with (interact indirectly with) LFA-1, where the interaction between layilin and LFA-1 is mediated by both directly binding to talin.

In some embodiments of the methods for treating cancer described herein, T cells (e.g., CD8⁺ T cells) may be modified ex vivo to increase the expression level of layilin. Modified T cells (e.g., modified CD8⁺ T cells) having a high expression level of layilin may be introduced into a subject having cancer (e.g., skin cancer) and accumulate in tissues (e.g., tumorous or cancerous tissues) to treat cancer (e.g., skin cancer).

In some embodiments of the methods for treating autoimmune disorders described herein, a layilin-binding protein (e.g., an anti-layilin antibody) may be used to disrupt layilin interactions or cell signaling involving layilin. Without being bound by any theory, a layilin-binding protein (e.g., an anti-layilin antibody), by disrupting layilin interactions or cell signaling involving layilin, may reduce T cell accumulation and/or T cell activity (e.g., autoreactive CD8⁺ T cells accumulation and/or autoreactive CD8⁺ T cells activity) in tissues, hence treating or ameliorating autoimmune disorders (e.g., autoimmune skin disorders (e.g., psoriasis)). As described in the Examples, the inventors have discovered that layilin colocalizes with LFA-1 and enhances LFA-1 activation on T cells to augment cellular adhesion. Thus, in methods for treating autoimmune disorders described herein, a layilin-binding protein may be used to interfere with the binding of a beta integrin complex expressed on CD8⁺ T cells to cell adhesion molecules and/or inhibit beta integrin complex activation.

### IV. Methods for Treating Cancer

The disclosure provides methods for treating cancer in a subject in need thereof by administering to the subject a modified T cell (e.g., a modified CD8⁺ T cell) having an increased layilin expression relative to an unmodified T cell (e.g., a wild-type CD8⁺ T cell). In some embodiments, the expression level of layilin in a modified T cell (e.g., a modified CD8⁺ T cell) is at least 10% higher (e.g., at least 15%, 20%, 25%, 30%, 35%, 40%, 45%, or 50%) than the expression level of layilin in an unmodified T cell (e.g., a wild-type CD8⁺ T cell) when measured under the same assay or experimental conditions. In some embodiments, the modified CD8⁺ T cell may be a CAR T cell. The disclosure also provides a modified chimeric antigen receptor (CAR) T cell comprising an increased layilin expression relative to an unmodified T cell. In some embodiments, the modified CAR T cell is CD8⁺. As demonstrated herein, high layilin expression is correlated with less cell mobility and more cell activation. Modified CD8⁺ T cells having high layilin expression may be introduced to the subject, such that the modified CD8⁺ T cells can accumulate in tumorous or cancer tissue to treat cancer.

In some embodiments, T cells (e.g., CD8⁺ T cells) may be isolated from the subject having cancer (e.g., autologous T cells). The isolated T cells (e.g., CD8⁺ T cells) may be modified ex vivo via one or more techniques described further herein (e.g., by transfection with an expression cassette comprising a nucleic acid encoding a layilin protein) to increase the expression of layilin. In some embodiments, the expression cassette may be placed in an expression vector. The modified T cells (e.g., modified CD8⁺ T cells) having high layilin expression may be further expanded ex vivo before being introduced into the subject. In some embodiments, the modified T cells (e.g., modified CD8⁺ T cells) having high layilin expression may be grown on a bioscaffold to the desired density or confluency before being introduced into the subject.

Furthermore, T cells (e.g., CD8⁺ T cells) may be isolated from the subject having cancer (e.g., autologous T cells). The isolated T cells (e.g., CD8⁺ T cells) may be modified to become CAR T cells. The chimeric antigen receptors on the surface of CAR T cells provide the cells the ability to target specific proteins, in particular, cancer antigens on the surface of cancer cells. Examples of cancer antigens include, but are not limited to, alphafetoprotein (AFP), carcinoembryonic antigen (CEA), CA-125, MUC-1, epithelial tumor antigen (ETA), tyrosinase, melanoma-associated antigen (MAGE), and p53. Various CAR T cells are known in the art, for example, as described in U.S. Pat. Nos. 9,499,629, 9,629,877, and 8,916,381 and US Patent Publication Nos. 20180112003 and 20180021418, each of which is incorporated herein by reference in its entirety. The CAR T cells (e.g., CD8⁺ CAR T cells) may be further modified to increase the expression of layilin. The CART cells (e.g., CD8⁺ CART cells) having high layilin expression may be further expanded ex vivo before being introduced into the subject. In some embodiments, the CAR T cells (e.g., CD8⁺ CAR T cells) having high layilin expression may be grown on a bioscaffold to the desired density or confluency before being introduced into the subject.

In some embodiments of the methods for treating cancer described herein, a layilin-binding protein (e.g., an anti-layilin antibody) may be used to enhance layilin interactions or cell signaling involving layilin. Without being bound by any theory, a layilin-binding protein (e.g., an anti-layilin antibody), by enhancing layilin interactions or cell signaling involving layilin, may increase T cell accumulation and/or T cell activity (e.g., anti-cancer CD8⁺ T cells accumulation and/or anti-cancer CD8⁺ T cells activity) in cancerous tissues, hence treating or ameliorating cancer. As described in the Examples, the inventors have discovered that layilin colocalizes with LFA-1 and enhances LFA-1 activation on T cells to augment cellular adhesion. Thus, in methods for treating cancer described herein, a layilin-binding protein may be used to promote the binding of a beta integrin complex expressed on CD8⁺ T cells to cell adhesion molecules and/or promote beta integrin complex activation.

Cancers that may be treated or ameliorated by methods described herein include, but are not limited to, skin cancer, bladder cancer, pancreatic cancer, lung cancer, liver cancer, ovarian cancer, colon cancer, stomach cancer, breast cancer, prostate cancer, renal cancer, testicular cancer, thyroid cancer, uterine cancer, rectal cancer, a cancer of the respiratory system, a cancer of the urinary system, oral cavity cancer, skin cancer, leukemia, sarcoma, carcinoma, basal cell carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, astrocytoma, oligoastrocytoma, biliary tract cancer, choriocarcinoma, CNS cancer, larynx cancer, small cell lung cancer, adenocarcinoma, giant (or oat) cell carcinoma, squamous cell carcinoma, anaplastic large cell lymphoma, non-small-cell lung cancer, neuroblastoma, rhabdomyosarcoma, neuroectodermal cancer, glioblastoma, breast carcinoma, inflammatory myofibroblastic tumor cancer, and soft tissue tumor cancer. In some embodiments, a cancer that may be treated or ameliorated by methods described herein is a metastatic cancer. In particular, a cancer that may be treated or ameliorated by methods described herein is skin cancer, such as melanoma (e.g., cutaneous melanoma).

### V. Methods for Treating Autoimmune Disorders

The disclosure provides methods for treating autoimmune disorders in a subject in need thereof, comprising administering to the subject a therapeutically effective amount of a layilin-binding protein. In some embodiments, the autoimmune disorder has a pathogenicity associated with the presence of CD8⁺ T cells in a diseased tissue (e.g., a diseased skin tissue). In other words, an autoimmune disorder can have a pathogenicity associated with an accumulation of CD8⁺ T cells (e.g., an accumulation of activated or autoreactive CD8⁺ T cells) in a diseased tissue (e.g., a diseased skin tissue). A diseased tissue may have an accumulation of CD8⁺ T cells (e.g., an accumulation of activated or autoreactive CD8⁺ T cells) that is greater than 10% (e.g., greater than 15%, 20%, 25%, 30%, 35%, 40%, 45%, or 50%) compared to the amount of CD8⁺ T cells present in a healthy tissue. The layilin-binding protein may disrupt layilin interactions or cell signaling involving layilin. Without being bound by any theory, a layilin-binding protein (e.g., an anti-layilin antibody) may reduce T cell accumulation (e.g., CD8⁺ T cells accumulation (e.g., autoreactive CD8⁺ T cells accumulation)) in tissues (e.g., diseased tissues), hence treating or ameliorating autoimmune disorders (e.g., autoimmune skin disorders (e.g., psoriasis)). As described in the Examples, the inventors have discovered that layilin enhances LFA-1 activation on T cells to augment cellular adhesion. Thus, in methods for treating autoimmune disorders described herein, a layilin-binding protein may be used to interfere with the binding of a beta integrin complex expressed on CD8+ T cells to cell adhesion molecules and/or inhibit beta integrin complex activation.

As demonstrated herein, it is discovered that psoriatic skin tissue contains highly activated CD8⁺ T cells expressing layilin at high levels, whereas normal skin tissue does not. Layilin expression may confer a selective advantage on CD8⁺ T cells to accumulate in tissues. Accordingly, without being bound by any theory, the accumulation of CD8⁺ T cell (e.g., autoreactive CD8⁺ T cells) in tissues can be prevented by targeting layilin on such T cells with a molecule that inhibits layilin interactions (i.e., a layilin-binding protein).

In other embodiments, the methods for treating autoimmune disorders in a subject in need thereof may include administering to the subject modified T cells (e.g., modified CD8⁺ T cells) that have a decreased layilin expression relative to unmodified T cells (e.g., wild-type CD8⁺ T cells). T cells (e.g., CD8⁺ T cells) may be modified ex vivo via one or more techniques described further herein (e.g., by nuclease-mediated genome editing) to decrease the expression of layilin.

Autoimmune disorders that may be treated or ameliorated by methods described herein include, but are not limited to, pemphigus vulgaris, pemphigus foliaceus, bullous pemphigoid, cicatricial pemphigoid, autoimmune alopecia, Graves' disease, Hashimoto's thyroiditis, autoimmune haemolytic anaemia, cryoglobulinemia, pernicious anaemia, myasthenia gravis, neuromyelitis optica, autoimmune epilepsy, encephalitis, autoimmune hepatitis, chronic autoimmune urticaria, linear IgA disease, IgA nephropathy, vitiligo, primary biliary cirrhosis, primary sclerosing cholangitis, autoimmune thrombocytopenic purpura, autoimmune Addison's disease, multiple sclerosis, Type 1 diabetes mellitus, dermatitis herpetiformis, coeliac disease, psoriasis, dermatomyositis, polymyositis, interstitial lung disease, Crohn's disease, ulcerative colitis, thyroid autoimmune disease, autoimmune uveitis, undifferentiated connective tissue disease, discoid lupus erythematosus, an immune-mediated inflammatory disease (IMID) such as scleroderma, rheumatoid arthritis or Sjogren's disease, an autoimmune connective tissue disease such as systemic lupus erythematosus, graft versus host disease, mixed connective tissue disease, atopic asthma, atopic dermatitis, Churg-Strauss vasculitis, allergic rhinitis, allergic eye disease, chronic non-autoimmune urticaria, and eosinophilic oesophagitis.

Methods for treating an autoimmune disorder described herein may be used to treat or ameliorate one or more symptoms of an autoimmune skin disorder. The immunological response associated with autoimmune disorders can destroy healthy tissue and cause tissue damage. Patients may experience short term or long-term symptoms including swelling, redness, a rash, hives, pustules, dryness, itching, and burst capillaries. Depending on the duration and severity of the symptoms, as well as the location of the lesions on the patient's body, autoimmune skin disorders can range from merely bothersome, mildly discomforting, to disfiguring. Further, autoimmune skin disorders can be painful.

In one embodiment of the present disclosure, the autoimmune disorder is an autoimmune disorder of the skin. The autoimmune skin disorder may be one or more of psoriasis, vitiligo, pemphigus vulgaris, pemphigus foliaceus, bullous pemphigoid, cicatricial pemphigoid, autoimmune alopecia, dermatitis herpetiformis, atopic dermatitis and chronic autoimmune urticaria. Accordingly, the invention provides methods for treating (decreasing or ameliorating one or more symptoms of) psoriasis, vitiligo, pemphigus vulgaris, pemphigus foliaceus, bullous pemphigoid, cicatricial pemphigoid, autoimmune alopecia, dermatitis herpetiformis, atopic dermatitis, and chronic autoimmune urticaria.

Methods for treating an autoimmune disorder described herein may be used to treat or ameliorate an autoimmune lung disorder (e.g., lung scleroderma). Methods for treating an autoimmune disorder described herein may be used to treat or ameliorate an autoimmune gut disorder (e.g., Crohn's disease, ulcerative colitis, or celiac disease).

### VI. Layilin-Binding Proteins

In methods for treating autoimmune disorders described herein, a layilin-binding protein (e.g., an anti-layilin antibody) may be used to disrupt layilin interactions or cell signaling involving layilin. Without being bound by any theory, a layilin-binding protein (e.g., an anti-layilin antibody), by disrupting layilin interactions or cell signaling involving layilin, may reduce T cell accumulation and/or T cell activity (e.g., autoreactive CD8⁺ T cells accumulation and/or autoreactive CD8⁺ T cells activity) in tissues, hence treating or ameliorating autoimmune disorders (e.g., autoimmune skin disorders (e.g., psoriasis)).

A layilin-binding protein may be an anti-layilin antibody or a fragment thereof. An anti-layilin antibody may be a full-length or intact antibody, a Fab, a F(ab′)₂, an Fv, a single chain Fv (scFv) antibody, a V_{H}, or a V_{H}H. In some embodiments, the anti-layilin antibody is a bispecific antibody, in which a first variable domain of the bispecific antibody binds to layilin and a second variable domain of the bispecific antibody binds to an antigen expressed on the CD8⁺ T cells (e.g., CD8⁺). In some embodiments, a layilin-binding protein (e.g., an anti-layilin antibody) may bind to a soluble fragment of layilin, a domain of layilin that binds to its natural ligand(s) e.g. hyaluronic acid or a fragment thereof, or a fragment or portion of the sequence of any one of SEQ ID NOS: 1-3 or 6-8. In some embodiments, a layilin-binding protein (e.g., an anti-layilin antibody) may bind to an epitope on a domain of layilin that binds to its natural ligand(s) e.g. hyaluronic acid.

Examples of anti-layilin antibodies include, but are not limited to, 3F7D7E2 (Sino Biological, mouse IgG1, immunogen=His-tagged human Layilin ECDaa1-220), Clone 7 (Sino Biological, mouse isotype not specified, immunogen=His-tagged human Layilin ECDaa1-220), Clone 8 (Sino Biological, mouse isotype not specified, His-tagged human Layilin ECDaa1-220), OTI4C11 (Novus Biologicals, mouse IgG1, immunogen=full-length human Layilin), 328024 (Novus Biologicals, mouse IgG1, immunogen=human Layilin ECDaa1-220); each of which is herein incorporated by reference in its entirety for all purposes. Anti-layilin antibodies can be blocking antibodies (also referred to as an antagonist antibody), e.g., blocking the interaction between layilin and a protein or other molecule. Anti-layilin antibodies can be cross-linking. Anti-layilin antibodies can be activating (also referred to as an agonist antibody). Anti-layilin antibodies can lead to depletion/clearance of a target, e.g., a cell expressing layilin.

In some embodiments, the layilin-binding protein inhibits the activity of layilin by interfering with the binding of a beta integrin complex such as LFA-1 expressed on CD8⁺ T cells to cell adhesion molecules such as ICAM-1 expressed on target cells e.g. cells of the skin and/or inhibits beta integrin complex (such as LFA-1) activation. In some embodiments, the layilin-binding protein enhances the activity of layilin e.g. it promotes the binding of a beta integrin complex expressed on CD8+ T cells to cell adhesion molecules and/or promotes beta integrin complex activation. A layilin-binding protein (e.g. antibody) that enhances the activity of layilin may, for example, be a cross-linking layilin-binding protein (e.g. antibody, particularly a full-length antibody).

In some embodiments, an anti-layilin antibody may be a monoclonal antibody. In other embodiments, an anti-layilin antibody may be a polyclonal antibody. In some embodiments, an anti-layilin antibody may be a chimeric antibody, an affinity matured antibody, a humanized antibody, or a human antibody. In certain embodiments, an anti-layilin antibody may be an antibody fragment, e.g., a Fab, a F(ab′)₂, an Fv, a single chain Fv (scFv) antibody, a V_{H}, or a V_{H}H.

In some embodiments, an anti-layilin antibody may be a chimeric antibody. For example, an antibody may contain antigen binding sequences from a non-human donor grafted to a heterologous non-human, human, or humanized sequence (e.g., framework and/or constant domain sequences). In one embodiment, the non-human donor may be a mouse. In another embodiment, an antigen binding sequence may be synthetic, e.g., obtained by mutagenesis (e.g., phage display screening, etc.). In a further embodiment, a chimeric antibody may have non-human (e.g., mouse) variable regions and human constant regions. In one example, a mouse light chain variable region may be fused to a human κ light chain. In another example, a mouse heavy chain variable region may be fused to a human IgG1 constant region.

An anti-layilin antibody may be generated using known techniques and methods in the art. Anti-layilin antibodies that are generated may be determined to inhibit or enhance layilin activity. An anti-layilin antibody that inhibits or prevents the activity of layilin is one that prevents or inhibits the binding of layilin to its natural ligand(s) e.g. hyaluronic acid and/or diminishes the binding of a beta integrin complex expressed on CD8+ T cells to cell adhesion molecules and/or diminishes beta integrin complex activation. An anti-layilin-binding protein that enhances the activity of layilin is one that promotes the binding of layilin to its natural ligand(s) e.g. hyaluronic acid and/or promotes the binding of a beta integrin complex expressed on CD8+ T cells to cell adhesion molecules and/or promotes beta integrin complex activation. Whether the generated anti-layilin antibody is one that inhibits or enhances layilin activity may be determined by means of assays, for example, layilin functional assays. Such functional assays are known in the art and may include, but may not be limited to, cell adhesion assays, fluorescent microscopy and/or flow cytometry. In some embodiments, antibodies are prepared by immunizing an animal or animals (e.g., mice, rabbits, or rats) with an antigen or a mixture of antigens for the induction of an antibody response. In some embodiments, the antigen or mixture of antigens is administered in conjugation with an adjuvant (e.g., Freund's adjuvant). A layilin protein or a fragment thereof (e.g., a soluble fragment of layilin; e.g., a domain of layilin that binds to its natural ligand(s) e.g. hyaluronic acid) may be used to immunize an animal. After an initial immunization, one or more subsequent booster injections of the antigen or antigens may be administered to improve antibody production. Following immunization, antigen-specific B cells are harvested, e.g., from the spleen and/or lymphoid tissue. Methods of preparing antibodies are described in, e.g., Delves et al., Antibody Production: Essential Techniques (2013), Wiley Sciences.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Optionally, phage or yeast display technology can be used to identify antibodies and Fab fragments that specifically bind to layilin and/or other selected antigen of a bispecific antibody. Techniques for the production of single chain antibodies or recombinant antibodies can also be adapted to produce antibodies. Antibodies can also be made bispecific, i.e., able to recognize two different antigens. Antibodies can also be heteroconjugates, e.g., two covalently joined antibodies.

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus. Methods of generating and screening hybridoma cell lines, including the selection and immunization of suitable animals, the isolation and fusion of appropriate cells to create the hybridomas, the screening of hybridomas for the secretion of desired antibodies, and characterization of the antibodies are known to one of ordinary skill in the art.

In some embodiments, the antibody is a chimeric antibody. Methods for making chimeric antibodies are known in the art. For example, chimeric antibodies can be made in which the antigen-binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody is a humanized antibody. Generally, a non-human antibody is humanized in order to reduce its immunogenicity. Humanized antibodies typically comprise one or more variable regions (e.g., CDRs) or portions thereof that are non-human (e.g., derived from a mouse variable region sequence), and possibly some framework regions or portions thereof that are non-human, and further comprise one or more constant regions that are derived from human antibody sequences. Methods for humanizing non-human antibodies are known in the art. Transgenic mice, or other organisms such as other mammals, can be used to express humanized or human antibodies. Other methods of humanizing antibodies include, for example, variable region resurfacing, CDR grafting, grafting specificity-determining residues (SDR), guided selection, and framework shuffling.

In some embodiments, antibody fragments (such as a Fab, a Fab′, a F(ab′)₂, a scFv, a V_{H}, a V_{HH}, or a diabody) are generated. Various techniques have been developed for the production of antibody fragments. Traditionally, these fragments were derived via proteolytic digestion of intact antibodies. However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments. According to another approach, F(ab′)₂ fragments can be isolated directly from recombinant host cell culture. Other techniques for the production of antibody fragments will be apparent to those skilled in the art.

### VII. Methods for Modifying T Cells

Methods for treating cancer or autoimmune disorders described herein may use modified T cells (e.g., modified CD8⁺ T cells). The T cells (e.g., CD8⁺ T cells) may be modified to increase or decrease layilin expression. In some embodiments, methods for treating cancer (e.g., a skin cancer) may use modified T cells (e.g., modified CD8⁺ T cells) that have an increased layilin expression relative to unmodified T cells (e.g., wild-type CD8⁺ T cells). In some embodiments, methods for treating an autoimmune disorder may use modified T cells (e.g., modified CD8⁺ T cells) that have a decreased layilin expression relative to unmodified T cells (e.g., wild-type CD8⁺ T cells). In some embodiments of the methods for treating cancer or autoimmune disorders described herein, T cells (e.g., CD8⁺ T cells) may first be isolated from the subject under treatment (e.g., autologous T cells) to undergo T cell modification ex vivo, then reintroduced into the subject. In other embodiments, T cells (e.g., CD8⁺ T cells) may be obtained from a donor to undergo T cell modification ex vivo, then introduced into the subject under treatment (e.g., heterologous T cells). In yet other embodiments, T cells (e.g., CD8⁺ T cells) may be obtained from a cell bank, modified ex vivo, then introduced into the subject under treatment.

Various methods and techniques are available to modify T cells (e.g., CD8⁺ T cells) to have an increased or decreased layilin expression relative to unmodified T cells (e.g., wild-type CD8⁺ T cells). In some embodiments, T cells (e.g., CD8⁺ T cells) may be modified by transfection with an expression vector containing an expression cassette comprising a nucleic acid encoding a layilin protein. In some embodiments, an expression cassette comprises a promoter operably linked to a polynucleotide encoding a layilin protein. In some embodiments, the promoter of the expression cassette is heterologous to the polynucleotide. In some embodiments, the promoter is inducible. In some embodiments, the promoter is tissue-specific (e.g., skin tissue-specific). Various transcription and translation control elements (e.g., promoter, transcription enhancers, transcription terminators, and the like) that may be used in an expression cassette are described further herein. In some embodiments, an expression cassette may be placed in an expression vector. In some embodiments, an expression vector may be a viral vector, such as viral vectors based on vaccinia virus, poliovirus, adenovirus, adeno-associated virus, SV40, herpes simplex virus, human immunodeficiency virus, and the like.

In other embodiments, a layilin nucleic acid sequence in a T cell (e.g., a CD8⁺ T cell) may be modified by a DNA nuclease, such as an engineered (e.g., programmable or targetable) DNA nuclease, to induce genome editing and hence increase or decrease the expression of the layilin nucleic acid sequence. Different nuclease-mediated genome editing techniques are described the subsections below.

In some embodiments, a nucleotide sequence encoding the DNA nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 polypeptide are commercially available from, e.g., Addgene, Life Technologies, Sigma-Aldrich, and Origene.

Depending on the target cell/expression system used, any of a number of transcription and translation control elements, including promoter, transcription enhancers, transcription terminators, and the like, may be used in an expression cassette, which may be placed in an expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

In other embodiments, a DNA nuclease may be introduced as a nucleotide. In some embodiments, a nucleotide sequence encoding a DNA nuclease may be present as an RNA (e.g., mRNA). The RNA can be produced by any method known to one of ordinary skill in the art. As non-limiting examples, the RNA can be chemically synthesized or in vitro transcribed. In certain embodiments, the RNA comprises an mRNA encoding a Cas nuclease such as a Cas9 polypeptide or a variant thereof. For example, the Cas9 mRNA can be generated through in vitro transcription of a template DNA sequence such as a linearized plasmid containing a Cas9 open reading frame (ORF). The Cas9 ORF can be codon optimized for expression in mammalian systems. In some instances, the Cas9 mRNA encodes a Cas9 polypeptide with an N- and/or C-terminal nuclear localization signal (NLS). In other instances, the Cas9 mRNA encodes a C-terminal HA epitope tag. In yet other instances, the Cas9 mRNA is capped, polyadenylated, and/or modified with 5-methylcytidine. Cas9 mRNA is commercially available from, e.g., TriLink BioTechnologies, Sigma-Aldrich, and Thermo Fisher Scientific.

In yet other embodiments, a DNA nuclease may be introduced as a polypeptide. The polypeptide can be produced by any method known to one of ordinary skill in the art. As non-limiting examples, the polypeptide can be chemically synthesized or in vitro translated. In certain embodiments, the polypeptide comprises a Cas protein such as a Cas9 protein or a variant thereof. For example, the Cas9 protein can be generated through in vitro translation of a Cas9 mRNA described herein. In some instances, the Cas protein such as a Cas9 protein or a variant thereof can be complexed with a single guide RNA (sgRNA) such as a modified sgRNA to form a ribonucleoprotein (RNP). Cas9 protein is commercially available from, e.g., PNA Bio (Thousand Oaks, Calif., USA) and Life Technologies (Carlsbad, Calif., USA).

CRISPR/Cas System

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “single guide RNA” or “sgRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas nuclease has DNA cleavage activity. The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. CRISPR-related endonucleases that are useful in the present invention are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983 or D986, as well as a mutation at H840 or N863. In some instances, the dCas9 enzyme contains a D10A or DION mutation. Also, the dCas9 enzyme can include a H840A, H840Y, or H840N. In some embodiments, the dCas9 enzyme of the present invention comprises D10A and H840A; D10A and H840Y; D10A and H840N; DION and H840A; D10N and H840Y; or DION and H840N substitutions. The substitutions can be conservative or non-conservative substitutions to render the Cas9 polypeptide catalytically inactive and able to bind to target DNA.

For genome editing methods, the Cas nuclease can be a Cas9 fusion protein such as a polypeptide comprising the catalytic domain of the type IIS restriction enzyme, FokI, linked to dCas9. The FokI-dCas9 fusion protein (fCas9) can use two guide RNAs to bind to a single strand of target DNA to generate a double-strand break.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) [also referred to as eSpCas9(1.0)], and SpCas9 (K848A/K1003A/R1060A) [also referred to as eSpCas9(1.1)] variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

In some embodiments, a CRISPR/Cas nuclease system may be used to gene edit T cells (e.g., CD8⁺ T cells) expressing layilin that were isolated from patients having cancer or an autoimmune disorder. In some embodiments, the CRISPR/Cas nuclease system may be used to increase the expression level of layilin in the T cells (e.g., CD8⁺ T cells) and the modified T cells may be used for cancer treatment. In other embodiments, the CRISPR/Cas nuclease system may be used to decrease the expression level of layilin in the T cells (e.g., CD8⁺ T cells) and the modified T cells may be used for treatment of an autoimmune disorder.

Other methods and techniques that can be used to modify T cells are available in the art. In one example, zinc finger nucleases (ZFNs) may be used. ZFNs are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. Examples of ZFNs include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140. In another example, TAL-effector nucleases (TALENS) may be used. TALENS are engineered transcription activator-like effector nucleases that contain a central domain of DNA-binding tandem repeats, a nuclear localization signal, and a C-terminal transcriptional activation domain. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55. In yet another example, meganucleases may be used. Meganucleases are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric. Detailed descriptions of useful meganucleases and their application in gene editing are found, e.g., in Silva et al., Curr Gene Ther, 2011, 11(1): 11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

### VIII. Introducing Expression Cassettes or Nuclease-Mediated Genome Editing Systems into Cells

Methods for introducing polypeptides, nucleic acids, and viral vectors (e.g., viral particles) into a target cell (e.g., a CD8⁺ T cell) are known in the art. Any known method can be used to introduce a polypeptide or a nucleic acid (e.g., a nucleotide sequence encoding the DNA nuclease or a modified sgRNA) into a target cell (e.g., a CD8⁺ T cell). Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Any known method can be used to introduce a viral vector (e.g., viral particle) into a target cell (e.g., a CD8⁺ T cell). In some embodiments, the homologous donor adeno-associated viral (AAV) vector described herein is introduced into a target cell (e.g., a CD8⁺ T cell) by viral transduction or infection. Useful methods for viral transduction are described in, e.g., Wang et al., Gene Therapy, 2003, 10: 2105-2111.

In some embodiments, the polypeptide and/or nucleic acids of the gene modification system can be introduced into a target cell (e.g., a CD8⁺ T cell) using a delivery system. In certain instances, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct) and one or more nucleic acid components such as an sgRNA and/or a donor template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

### IX. Methods for Cell Expansion

Modified T cells (e.g., modified CD8⁺ T cells) having an increased or decreased layilin expression relative to unmodified T cells (e.g., wild-type CD8⁺ T cells) may be expanded ex vivo. For example, modified T cells (e.g., modified CD8⁺ T cells) may be cultured by embedding the cells in a bioscaffold. A bioscaffold refers to a substrate or matrix on which cells can grow and may be derived from or made from natural or synthetic tissues or cells or other natural or synthetic materials. In some embodiments, a bioscaffold may be derived from, made from, and/or comprises natural or synthetic materials such as extracellular matrix, collagen Type I, collagen Type IV, fibronectin, polycarbonate, and polystyrene. In some embodiments, a bioscaffold may include a decellularized extracellular matrix (ECM) membrane. A bioscaffold may be used for tissue or cell engineering and/or ex vivo expansion or regeneration. A bioscaffold may be in the form of a membrane, a matrix, a microbead, or a gel (e.g., a hydrogel), and/or a combination thereof. A bioscaffold can be made out of materials that have the physical or mechanical attributes required for grafting or implantation. In some embodiments, the bioscaffold is made of a semi-permeable material which may include collagen (e.g., collagen Type-I, collagen Type-IV), which may be cross-linked or uncross-linked. The bioscaffold may also include polypeptides or proteins obtained from natural sources or by synthesis, such as small intestine submucosa (SIS), peritoneum, pericardium, polylactic acids and related acids, blood (i.e., which is a circulating tissue including a fluid portion (plasma) with suspended formed elements (red blood cells, white blood cells, platelets)), or other materials that are bioresorbable (e.g., bioabsorbable polymers, such as elastin, fibrin, laminin, and fibronectin).

A bioscaffold may have one or several surfaces, such as a porous surface, a dense surface, or a combination of both. The bioscaffold may also include semi-permeable, impermeable, or fully permeable surfaces. The bioscaffold may be autologous or allogeneic. A bioscaffold may be a solid, semi-solid, gel, or gel-like scaffold characterized by being able to hold a stable form for a period of time to enable the adherence and/or growth of cells thereon, both before grafting and after grafting, and to provide a system similar to the natural environment of the cells to optimize cell growth. Some examples of bioscaffolds include, but are not limited to, Vitrogen™, a collagen-containing solution which gels to form a cell-populated matrix, and the connective-tissue scaffolds described in US Patent Publication No. 20040267362). A bioscaffold can be cut or formed into any regular or irregular shape. In some embodiments, the bioscaffold can be cut to correspond to the shape of the area where it is to be grafted. The bioscaffold can be flat, round, and/or cylindrical in shape. In some embodiments, a bioscaffold may include type I/III collagen (e.g., collagen Type-I). In some embodiments, a bioscaffold may include small intestinal submucosa.

In some embodiments, a bioscaffold may be a decellularized ECM membrane. A decelluarlized ECM membrane may include collagen (e.g., collagen Type-I), elastic fibers, glycosoaminoglycans, proteoglycans, and adhesive glycoproteins. The decellularized ECM membrane serves as a network or scaffold supporting the attachment and proliferation of the modified T cells (e.g., modified CD8⁺ T cells). The decellularized ECM membrane may mimic the microenvironment of the tissue or organ.

A bioscaffold may be derived from a mammalian tissue source, such as a tissue from human, monkey, pig, cow, sheep, horse, goat, mouse, and rat. The tissue source from which to make the bioscaffold may be from any organ or tissue of a mammal, including without limitation, intestine tissue, pancreas tissue, liver tissue, lung tissue, trachea tissue, esophagus tissue, kidney tissue, bladder tissue, skin tissue, heart tissue, brain tissue, placenta tissue, and umbilical cord tissue. Further, a bioscaffold may include any tissue obtained from an organ, including, for example and without limitation, submucosa, epithelial basement membrane, and tunica propria. In some embodiments, a bioscaffold may be made from small intestinal submucosal (SIS) membrane.

A bioscaffold can have suitable viscoelasticity, flow behavior, and thickness for grafting or injecting to the desired area (e.g., skin) for clinical treatment. In some embodiments, a bioscaffold can contain components that are present in tissue from which it was derived. In certain embodiments, the bioscaffold can contain components that are present in a skin to mimic the characteristics of the skin tissue and its organization and function. For example, and not by way of limitation, the bioscaffold can include collagen (e.g., collagen Type-I), glycosaminoglycan, laminin, elastin, non-collagenous protein and the like.

Techniques and methods of culturing cells in a bioscaffold for grafting purposes are known in the art. An optimal plating density to achieve a certain percentage of coverage in a certain period of time may be determined by a skilled artisan. Depending on the number of days before the expanded cells are used for grafting, the plating density may be adjusted accordingly to achieve the desired number of cells and the percentage of coverage in the bioscaffold for grafting.

Methods of preparing a bioscaffold are known in the art. Examples of methods of preparing a bioscaffold are described in, e.g., U.S. Patent Application Publication Nos. 2004/0076657, 2003/0014126, 20050191281, 2005/0256588, and U.S. Pat. Nos. 6,933,103, 6,743,574, 6,734,018, 5,855,620, each of which is incorporated herein by reference in its entirety.

### X. Pharmaceutical Compositions

A pharmaceutical composition for use in methods for treating an autoimmune disorder or cancer in a subject as described herein may include a layilin-binding protein (e.g., an anti-layilin antibody) or modified T cells (e.g., modified CD8⁺ T cells) having an increased or decreased layilin expression relative to unmodified T cells (e.g., wild-type CD8⁺ T cells), respectively. In some embodiments, a pharmaceutical composition for use in methods for treating cancer in a subject as described herein may include modified T cells (e.g., modified CD8⁺ T cells) having an increased layilin expression relative to unmodified T cells (e.g., wild-type CD8⁺ T cells). In some embodiments, a pharmaceutical composition for use in methods for treating an autoimmune disorder in a subject as described herein may include a layilin-binding protein (e.g., an anti-layilin antibody). In other embodiments, a pharmaceutical composition for use in methods for treating an autoimmune disorder in a subject as described herein may include modified T cells (e.g., modified CD8⁺ T cells) having a decreased layilin expression relative to unmodified T cells (e.g., wild-type CD8⁺ T cells).

Pharmaceutical compositions typically must be sterile and stable under the conditions of manufacture and storage. Pharmaceutical compositions of the disclosure may comprise additional active ingredients. In therapeutic applications, compounds may be administered to a subject already suffering from a disorder or condition as described herein, in an amount sufficient to cure, alleviate, or partially arrest the condition or one or more of its symptoms. Such therapeutic treatment may result in a decrease in the severity of disease symptoms, or an increase in frequency or duration of symptom-free periods.

In some embodiments, in particular in respect of treatments for autoimmune disorders, a pharmaceutical composition may further include other agents, such as immunosuppressants, to be used in a combination therapy. Examples of immunosuppressants include, but are not limited to, corticosteroids (e.g., prednisone, budesonide, and prednisolone), kinase inhibitors (e.g., tofacitinib), calcineurin inhibitors (e.g., cyclosporine and tacrolimus), mTOR inhibitors (e.g., sirolimus and everolimus), IMDH inhibitors (e.g., azathioprine, leflunomide, and mycophenolate), and other biologics (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab, and daclizumab).

In some embodiments, a pharmaceutical composition may further include other agents, such as anti-cancer agents, to be used in a combination therapy. Examples of anti-cancer agents include, but are not limited to, an anti-PD-1 antibody (e.g., nivolumab, pembrolizumab), an anti-CTLA-4 (cytotoxic T-lymphocyte-associated protein 4) antibody, and an anti-LAG3 antibody. Other examples of anti-cancer agents include, but are not limited to, alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CBI-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Nicolaou et al. Angew. Chem Intl. Ed. Engl., 33: 183-186 (1994)); CDP323, an oral alpha-4 integrin inhibitor; dynemicin, including dynemicin A; an esperamicin; neocarzinostatin chromophore and related chromoprotein enediyne antibiotic chromophores), aclacinomysins, actinomycin, authramycin, azaserine, bleomycin, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, 5-azacytidine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINEO, FILDESINO); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Further examples of anti-cancer agents include, but are not limited to, cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, methotrexate, 6-mercaptopurine, fludarabine, 5-azacytidine, pentostatin, cytarabine, gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, or docetaxel.

In addition, the pharmaceutical composition may contain one or more pharmaceutically acceptable carriers or excipients, which can be formulated by methods known to those skilled in the art. Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions may be administered parenterally in the form of an injectable formulation. Pharmaceutical compositions for injection (i.e., intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

The pharmaceutical composition may be formed in a unit dose form as needed. The amount of active component, e.g., a layilin-binding protein (e.g., an anti-layilin antibody), included in the pharmaceutical preparations is such that a suitable dose within the designated range is provided (e.g., a dose within the range of 0.01-500 mg/kg of body weight).

### XI. Administration, Routes, and Dosage

Pharmaceutical compositions described herein may be formulated for subcutaneous administration, intramuscular administration, intravenous administration, parenteral administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. In some embodiments, pharmaceutical compositions may administered locally or systemically (e.g., locally). In particular embodiments, pharmaceutical compositions may be administered locally at the affected area, such as skin or cancerous tissue.

The dosage of the pharmaceutical compositions depends on factors including the route of administration, the disease to be treated, and physical characteristics, e.g., age, weight, general health, of the subject. In some embodiments, the amount of active ingredient (e.g., a layilin-binding protein (e.g., an anti-layilin antibody) or modified T cells (e.g., modified CD8⁺ T cells)) contained within a single dose may be an amount that effectively prevents, delays, or treats the disease without inducing significant toxicity. The dosage may be adapted by the physician in accordance with conventional factors such as the extent of the disease and different parameters of the subject.

The pharmaceutical compositions may be administered in a manner compatible with the dosage formulation and in such amount as is therapeutically effective to result in an improvement or remediation of the symptoms. The pharmaceutical compositions may be administered in a variety of dosage forms, e.g., subcutaneous dosage forms, intravenous dosage forms, and oral dosage forms (e.g., ingestible solutions, drug release capsules). Pharmaceutical compositions containing the active ingredient (e.g., a layilin-binding protein (e.g., an anti-layilin antibody) or modified T cells (e.g., modified CD8⁺ T cells)) may be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines.

### XII. Methods for Identifying Modulators of Layilin and Beta-Integrin Complexes

The compositions and methods described herein or presented in the examples herein can be used to identify modulators that alter interaction between any of the compositions described herein, such as any of the proteins (e.g., layilin, layilin ligands, constituents of layilin complexes, beta-integrin complexes or constituents thereof, any of the antibodies described herein), molecules, or compounds (e.g., hyaluronic acid) described herein. The compositions and methods described or presented in the examples herein can be used to identify modulators of layilin interaction with its ligand or member of a complex that can have layilin present. The compositions and methods described or presented in the examples herein can be used to identify modulators of beta-integrin complexes (e.g., LFA-1) interaction with a ligand of the complex or member of a constituent in the complex. Modulators include but are not limited to binding reagents (e.g., antibodies or antigen binding fragments thereof), an RNAi nucleic acid (e.g., siRNAs, miRNAs, antisense oligonucleotides, shRNAs, etc.), a genome editing system (e.g., a nuclease genomic editing system, a transposon system, viral vector editing platforms, etc.), and a small molecule (e.g., a small molecule inhibitor). A nuclease genomic editing system can use a variety of nucleases to cut a target genomic locus, including, but not limited to, a Transcription activator-like effector nuclease (TALEN) or derivative thereof, a homing endonuclease (HE) or derivative thereof, a zinc-finger nuclease (ZFN) or derivative thereof, or any of the CRISPR-based systems described herein.

Modulators can be identified using an assay, such as a binding assay (e.g., any of the binding assays methods described herein or presented in the examples herein). Examples of binding assays include, but are not limited to ELISAs (e.g., a competition ELISA), proximity ligation assays, biosensor assays (e.g., surface plasmon resonance and interferometry assays), flow cytometry, immunohistochemistry, and cell adhesion assays. Binding activity may be determined, for example, by competition for binding to the binding domain of the cognate molecule (i.e. competitive binding assays). Competitive binding assays can be performed using standard methodology. One configuration of a competitive binding assay for a recombinant fusion protein comprising a ligand uses a labeled (e.g., radio-, enzyme-, chromogen-, or fluorochrome-labeled, soluble receptor as a competing binder, and intact cells expressing a native form of the ligand. The binding of the recombinant fusion protein can be determined by measuring a decrease in binding to the cells by the labeled, soluble receptor. Similarly, a competitive assay for a recombinant fusion protein comprising a receptor uses a labeled, soluble ligand, and intact cells expressing a native form of the receptor. Instead of intact cells expressing a native form of the cognate molecule, one could substitute purified cognate molecule bound to a solid phase. Qualitative or semi-quantitative results can be obtained by standard methodology (e.g., competitive binding assays, colorimetric assay, ELISA, or flow cytometry). Scatchard plots, linear regression, or nonlinear regression may be utilized to generate quantitative results. Assays can be used to determine increased binding, e.g., assays designed to determine allosteric activation by a modulator. Modulators can also be identified and/or assessed using other assays known in the art, such as assays that measure biological activity (e.g., proliferation, killing, activation, cytokine secretion, integrin activation, cell adhesion, etc.).

### EXAMPLES

Statistical analyses were performed with Prism software (GraphPad). For wet laboratory experiments, a two-tailed unpaired Student's t-test or two way ANOVA were used to calculate P values and appropriate statistical analysis assuming a normal sample distribution was applied, as indicated. RNA-seq experiments were analyzed as described in the above section. All experiments were performed with at least 2 independent trials, as indicated. P values correlate with symbols as follows: ns=not significant; *p<0.05; **p<0.01; ***p<0.001; ****p<0.0001.

### Example 1 Expression of Layilin on Activated CD8⁺ T Cells

Methods and Materials

Human PBMCs from two individual donors were purchased from AllCells (Alameda, Calif.). CD8+ T cells were enriched from these samples using a negative selection kit (STEMCELL Technologies). Isolated T cells were activated with αCD3/CD28 ImmunoCult™ reagent and grown in ImmunoCult™-XF T cell Expansion Medium (STEMCELL Technologies) with the addition of 10 ng/mL IL-15 and 100 U/mL IL-2.

Single-cell suspensions prepared as described above were stained with Ghost 510 Viability dye (Tonbo Biosciences) in PBS. Following a wash step, cells were stained for surface markers in PBS with 2% FCS. For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

Fluorophore conjugated antibodies specific for mouse and human antigens were purchased from eBioscience, BD Biosciences, and Biolegend. The following clones were used for staining human cells: α-layilin (clone 3F7D7E2) and α-CD8a (clone SK1). The α-layilin antibody was conjugated to biotin using the One-step Antibody Biotinylation Kit (Miltenyi Biotec, catalog no. 130-093-385) and detected with Streptavidin-Phycoerythrin (PE) (Biolegend).

Results

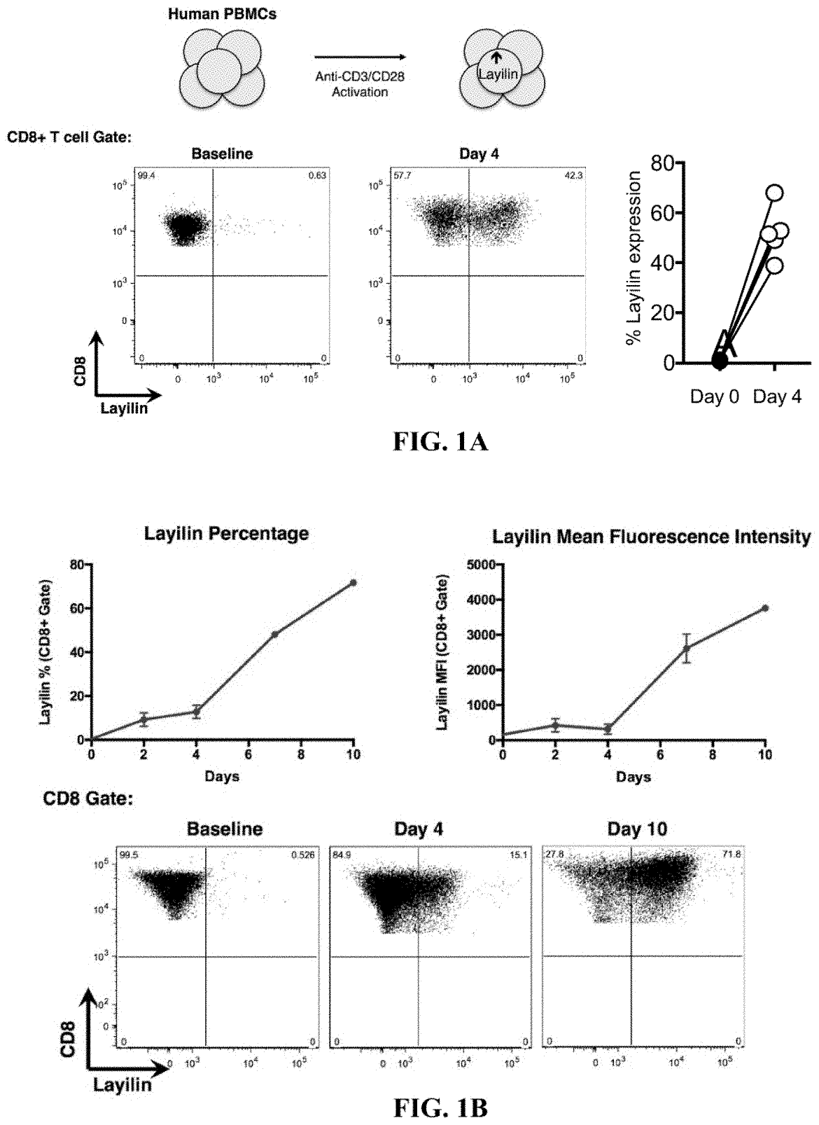

CD8⁺ T cells were purified from human peripheral blood samples and either left untreated (baseline) or treated for up to 10 days with anti-CD3 and anti-CD28 coated beads to induce T cell activation through the T cell receptor and the costimulatory receptor CD28. As shown in FIG. 1A, flow cytometric quantification of layilin protein expression revealed negligible levels on freshly isolated naive CD8⁺ T cells at baseline. However, 4-days after activation, approximately 50% of these cells expressed appreciable levels of layilin on the cell surface. FIG. 1B further shows the kinetics (Days 0, 2, 4, 7, and 10) of layilin expression on human peripheral blood CD8⁺ T cells after activation with anti-CD3 and anti-CD28 coated beads for a separate set of patient derived samples. Accordingly, the results demonstrate layilin was highly expressed on peripheral blood CD8⁺ T cells after activation through the T cell receptor.

### Example 2 Expression of Layilin on CD8⁺ T Cells in Lesional Skin

Methods and Materials

Single cell suspensions were obtained from 4 mm punch biopsies of psoriatic lesions (defined as a clinically inflamed psoriatic plaque) and non-lesional skin (defined as >10 cm away from a lesional psoriatic plaque in the same anatomic location) from 4 patients with active cutaneous psoriasis. Cells were first washed with 5 mM EDTA-PBS and centrifuged at 600 g for 5 minutes at 4° C. Cells were then resuspended with equal volumes of 5 mM EDTA-PBS and 50 uM cisplatin (Sigma, P4394) for 1 minute at room temperature (RT) before quenching with 5 mM EDTA-PBS with 0.5% BSA. After centrifugation, cells were fixed with 1.6% PFA in PBS with 0.5% BSA and 5 mM EDTA for 10 minutes at RT and then washed twice with PBS. Cells were then resuspended in PBS with 0.5% BSA and 10% DMSO and stored at −80° C. Prior to staining, cells were left to thaw at RT and washed in Cell Staining Media (CSM, PBS with 0.5% BSA and 0.02% NaN3) and then vortexed with FC Receptor Blocking Solution (BioLegend, 422302). LAYN (Sino Biological, 10208-MM02), PD-1 (BioLegend, EH12.2H7), and CD8a (BioLegend, RPA-T8) antibodies were metal-conjugated at the UCSF Parnassus Flow Cytometry Core using Maxpar Antibody Labeling Kits (Fluidigm). All other metal conjugated antibodies were obtained from Fluidigm. Cells were stained as previously described (Spitzer et al., 2015). Briefly, cells were stained in an extracellular antibody cocktail for 30 minutes at RT on a shaker and then washed with CSM. Cells were then permeabilized with the Foxp3/Transcription Factor Staining Buffer Set (eBioscience, 00-5523-00) for 30 minutes at RT on a shaker and then washed twice with Permeabilization Buffer (eBioscience, 00-8333-56) before staining in an intracellular antibody cocktail for 1 hour at RT on a shaker. Following intracellular staining, cells were washed once with Permeabilization Buffer and once with CSM, and then resuspended in PBS with 1.6% PFA and 100 nM Cell-ID Intercalator-Ir (Fluidigm, 201192B) and kept at 4° C. Before data acquisition, cells were washed sequentially in CSM, PBS, and MilliQ H₂O. Cells were then resuspended in MilliQ H₂O containing EQ Four Elements Calibration Beads (Fludigm, 201078) and analyzed with a CyTOF2 Mass Cytometer (Fluidigm). Mass cytometry files were normalized to the bead standards (Finck et al., 2013) in R (3.6.1) using the premessa package (0.2.4, github.com/ParkerlCI/premesa). Analysis was performed on viable singlets as determined by the iridium, event length, and cisplatin channels. t-SNE analysis was performed as described in Kalekar et al. (Sci Immunol. 2019 Sep. 6; 4(39). pii: eaaw2910. doi: 10.1126/sciimmunol.aaw2910), herein incorporated by reference for all purposes.

Results

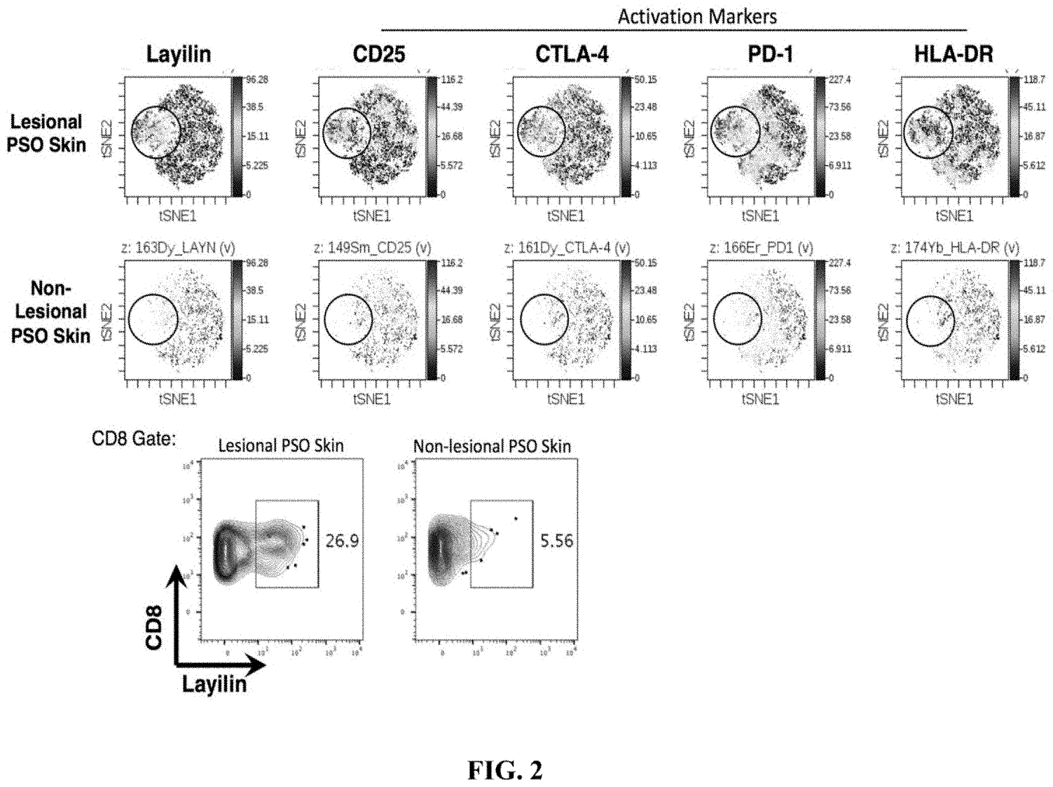

Single cell suspensions from lesional and non-lesional skin were obtained from 4 patients with active cutaneous psoriasis and stained for cell surface protein expression using CyTOF, as described above. Data in top 2 rows of FIG. 2 is displayed as a dimensionally reduced t-SNE (t-distributed stochastic neighbor embedding) plot of layilin protein expression and activation protein (CD25, CTLA-4, PD-1, and HLA-DR) expression on CD8⁺ T cells from lesional skin and non-lesional skin combined from 4 patients. Data shows the presence of a highly activated CD8⁺ T cell subset (circled populations) in lesional psoriatic (PSO) skin that was relatively absent in non-lesional psoriatic skin. This highly activated CD8⁺ T cell subset expressed high levels of layilin. Bottom row shows a representative example of a CyTOF contour plot showing high levels of layilin expression on CD8⁺ T cells in lesional psoriatic skin compared to non-lesional skin from a single patient.

### Example 3 Effects of Layilin Expression on Tumor Regression

Methods and Materials

Rag2^{-/-} and Ptprc^{a} (CD45.1) animals were purchased from The Jackson Laboratory (Bar Harbor, Me.) while the E8I^{Cre} strain was a gift from Dr. Shomyseh Sanjabi at University of California, San Francisco (UCSF). Germline Layn^{-/-} and Layn^{f/f} mice were created using a CRISPR-Cas9 approach. Guide RNAs were designed to introduce either a premature stop codon into exon 4 (Layn^{-/-}) or a complete exon 4 deletion and delivered with Cas9 into C57BL/6 embryos. Founder pups were backcrossed to wildtype C57BL/6 mice. All animal experiments were performed on littermate age and gender matched 8-20 week old mice maintained through routine breeding at the UCSF School of Medicine in a specific pathogen free facility. Experimental procedures were approved by IACUC and performed in accordance with guidelines established by the Laboratory Animal Resource Center (LARC) at UCSF. MC38-LUC2 and B16.F10 cell lines were provided by Dr. Jeffrey Bluestone (UCSF) and verified to be mycoplasma free. 1×10⁵ B16.F10 or 5×10⁵ MC38 cells were injected subcutaneously. Tumor growth was measured either manually with calipers or bioluminescence IVIS imaging, as indicated. Tumor volume was calculated according to the formula V=(W²*L)/2 (A. Faustino-Rocha et al., Estimation of rat mammary tumor volume using caliper and ultrasonography measurements. Lab Anim. (NY). 42, 217-224 (2013)). For adoptive transfer experiments 2.5×10⁵ CD4⁺ and 7.5×10⁵ CD8⁺ T cells from CD8^{Cre}LAYN^{f/f} were co-injected intravenously with equal ratios of wildtype Ptprc^{a} T cells two days prior to tumor challenge.

Single cell suspensions of mouse tumors were obtained by finely mincing tissues and digesting in a buffer cocktail containing collagenase XI, DNase, and hyaluronidase in complete RPMI for 45 minutes in an 37° C. incubator shaker at 225 rpm. Tissue samples were then vortexed and strained through a 100 μm filter and the resulting flow through washed and pelleted for flow cytometric analysis.

Single-cell suspensions prepared as described above were stained with Ghost 510 Viability dye (Tonbo Biosciences) in PBS. Following a wash step, cells were stained for surface markers in PBS with 2% FCS. For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

Fluorophore conjugated antibodies specific for mouse and human antigens were purchased from eBioscience, BD Biosciences, and Biolegend. Antibodies for staining mouse cells: α-CD8a (clone 53-6.7); α-TCR-f3 (clone H57-597); α-CD45.1 (clone A20); α-CD45.2 (clone 104).

Results

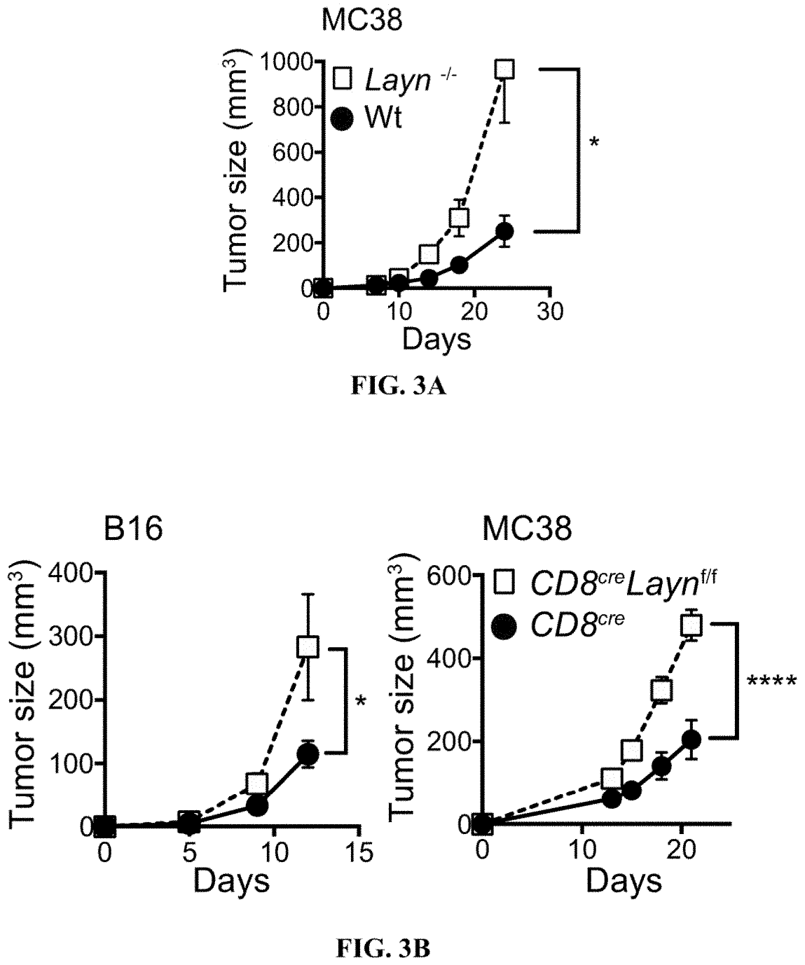

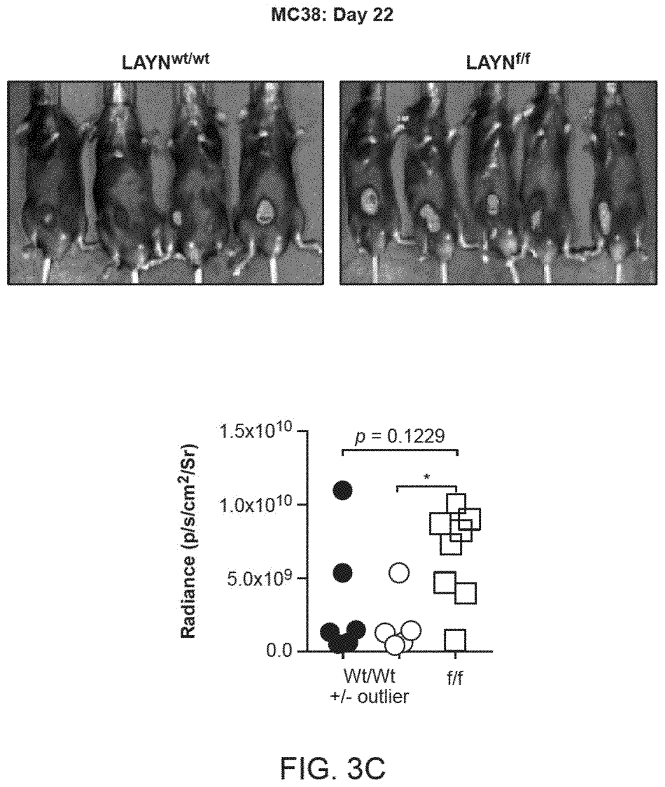

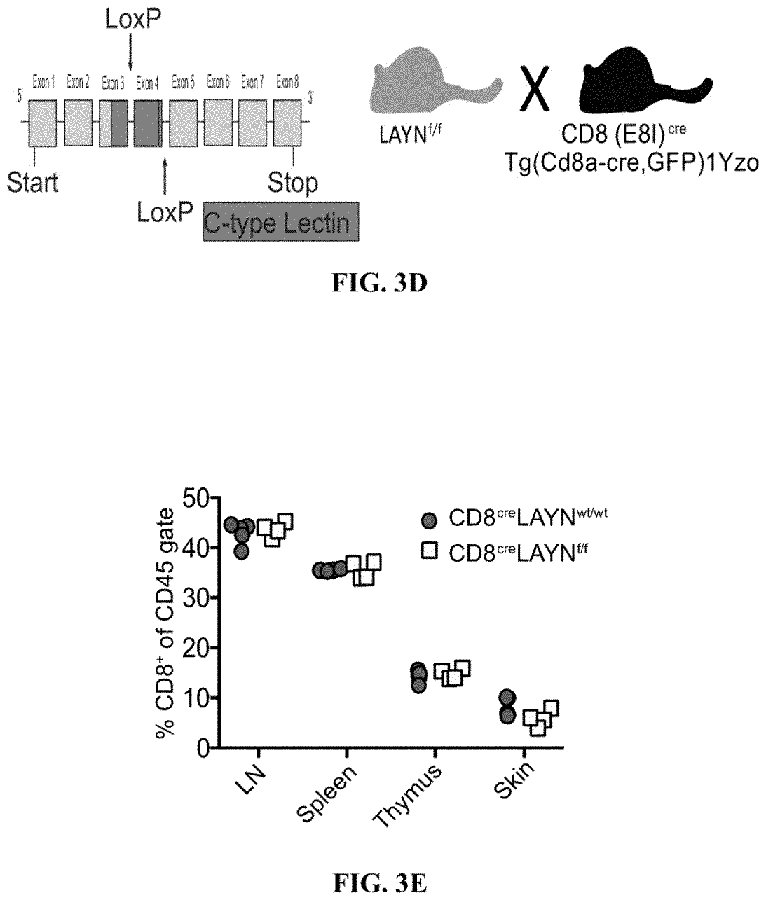

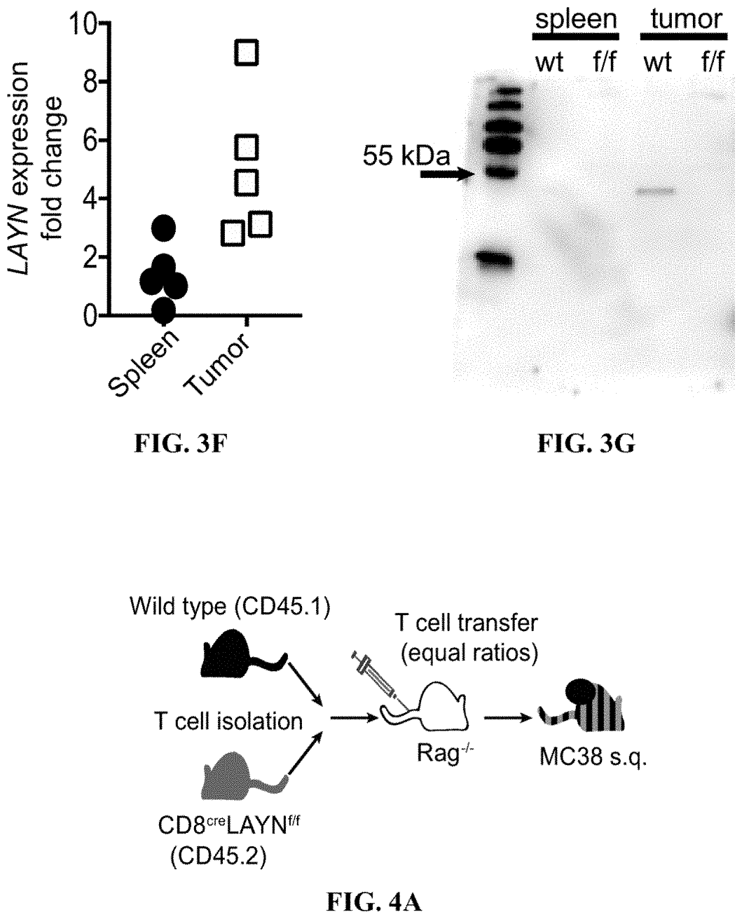

To examine the functional role of layilin on CD8⁺ T cell mediated anti-tumor immunity, a germline Layn knockout mouse strain as well a strain in which Layn could be conditionally deleted in specific cell types was generated (i.e., Layn^{flox/flox} mice). FIG. 3D illustrates the general strategy for the conditional deletion of Layn. Flox sequences were inserted to flank exon 4 of the layilin gene using CRISPR/Cas9 technology (Cong et al., 2013). This results in complete deletion of exon 4, corresponding to the C-type lectin domain of LAYN, when crossed to mice expressing Cre-recombinase in specific cell lineages (Borowsky and Hynes, 1998).

To elucidate the function of layilin on TILs, MC38 adenocarcinoma was transplanted into Layn^{-/-} or wildtype control mice and the kinetics of tumor growth were measured. Layilin-deficient animals demonstrated increased tumor growth (FIG. 3A). To determine if layilin-expressing CD8⁺ TILs play a role in limiting tumor growth, Layn was specifically deleted in CD8⁺ T cells by crossing Layn^{f/f} mice to a CD8^{cre} (E8I^{cre}) strain where Cre-recombinase activity is present only in post-thymic CD8⁺ cells (Zou et al., 2001). At steady-state, these mice exhibited normal CD8 frequencies across multiple organs (FIG. 3E). CD8^{cre}Layn^{f/f} mice were utilized in two separate tumor models. Either B16-F10 melanoma or MC38 cell lines into CD8^{cre}Layn^{wt/wt} or CD8^{cre}Layn^{f/f} mice and layilin expression and tumor growth kinetics was quantified. Layilin deletion on CD8⁺ T cells resulted in enhanced tumor growth in both the B16-F10 model (FIG. 3B, left panel) and MC38 model (FIG. 3B, right panel) by caliper quantification of tumor size. Layilin deletion on CD8⁺ T cells resulted in enhanced tumor growth in the MC38 model by quantification of bioluminescence (FIG. 3C—images top panel; quantification bottom panel). CD8⁺ T cells purified from MC38 tumors growing in CD8^{cre}Layn^{wt/wt} hosts had increased expression of layilin at the mRNA and protein levels when compared to their splenic counterparts, and layilin expression was absent in CD8^{cre}Layn^{f/f} animals (FIGS. 3F and G). Taken together, these results suggest that layilin expression is increased on murine CD8⁺ T cells in the tumor microenvironment and that expression of this protein on TILs results in reduced tumor growth.

### Example 4 Effects of Layilin Expression on CD8⁺ T Cell Accumulation

Methods and Materials

Rag2^{-/-} and Ptprc^{a} (CD45.1) animals were purchased from The Jackson Laboratory (Bar Harbor, Me.) while the E8I^{Cre} strain was a gift from Dr. Shomyseh Sanjabi at University of California, San Francisco (UCSF). Germline Layn^{-/-} and Layn^{f/f} mice were created using a CRISPR-Cas9 approach. Guide RNAs were designed to introduce either a premature stop codon into exon 4 (Layn^{-/-}) or a complete exon 4 deletion and delivered with Cas9 into C57BL/6 embryos. Founder pups were backcrossed to wildtype C57BL/6 mice. All animal experiments were performed on littermate age and gender matched 8-20 week old mice maintained through routine breeding at the UCSF School of Medicine in a specific pathogen free facility. Experimental procedures were approved by IACUC and performed in accordance with guidelines established by the Laboratory Animal Resource Center (LARC) at UCSF. MC38-LUC2 cell lines were provided by Dr. Jeffrey Bluestone (UCSF) and verified to be mycoplasma free. 5×10⁵ MC38 cells were injected subcutaneously. For adoptive transfer experiments 2.5×10⁵ CD4⁺ and 7.5×10⁵ CD8⁺ T cells from CD8^{Cre}LAYN^{f/f} were co-injected intravenously with equal ratios of wildtype Ptprc^{a} T cells two days prior to tumor challenge.

Single cell suspensions of mouse tumors were obtained by finely mincing tissues and digesting in a buffer cocktail containing collagenase XI, DNase, and hyaluronidase in complete RPMI for 45 minutes in an 37° C. incubator shaker at 225 rpm. Tissue samples were then vortexed and strained through a 100 μm filter and the resulting flow through washed and pelleted for flow cytometric analysis.

Single-cell suspensions prepared as described above were stained with Ghost 510 Viability dye (Tonbo Biosciences) in PBS. Following a wash step, cells were stained for surface markers in PBS with 2% FCS. For intracellular staining, cells were fixed and permeabilized with the Foxp3/Transcription Factor Staining Buffer Set (eBiosciences, catalog 00-5523-00). For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

Fluorophore conjugated antibodies specific for mouse and human antigens were purchased from eBioscience, BD Biosciences, and Biolegend. Antibodies for staining mouse cells: α-CD8α (clone 53-6.7); α-TCR-f3 (clone H57-597); α-CD4 (clone GK1.5); α-CD45.1 (clone A20); α-CD45.2 (clone 104); α-Ki67 (clone B56); α-IFNγ (clone XMG1.2); α-TNFα (clone MP6-XT22); α-granzyme B (clone GB11); α-PD-1 (clone 29F.1A12).

Results

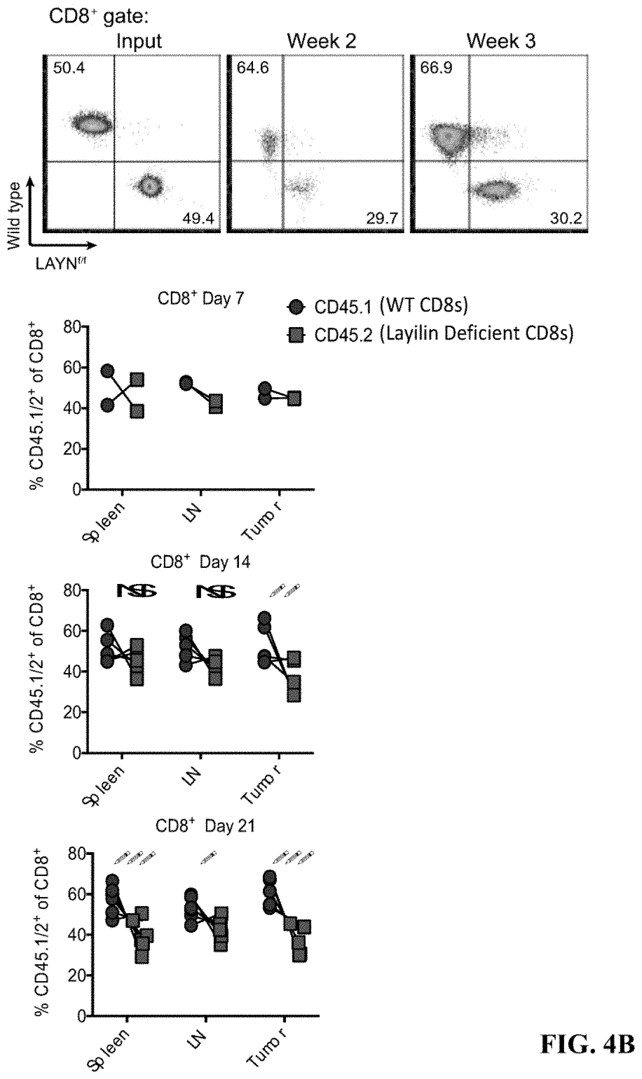

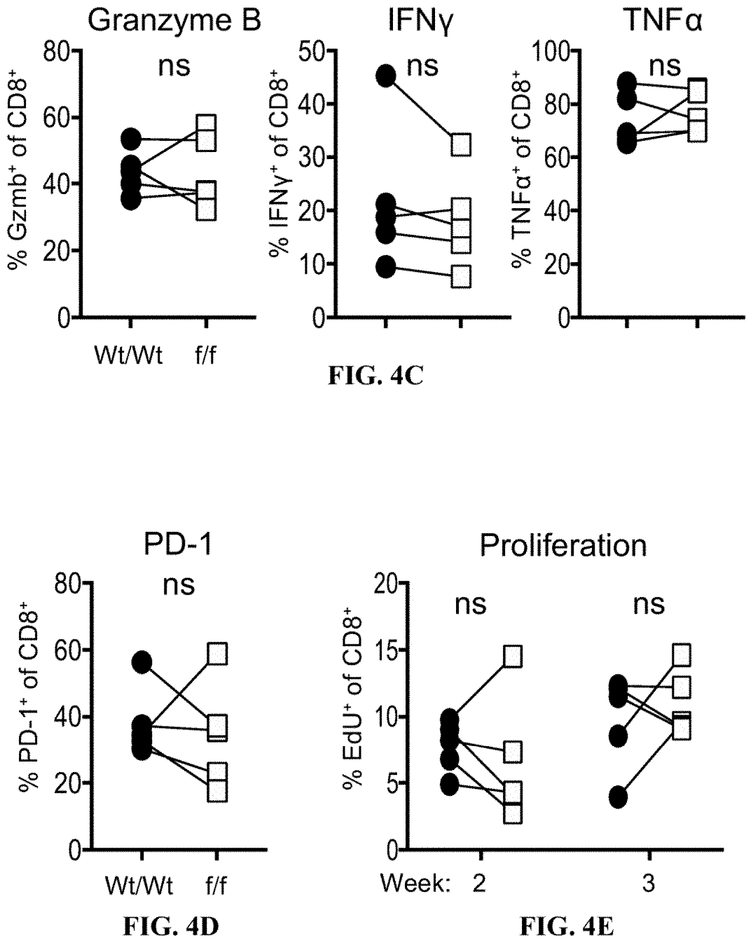

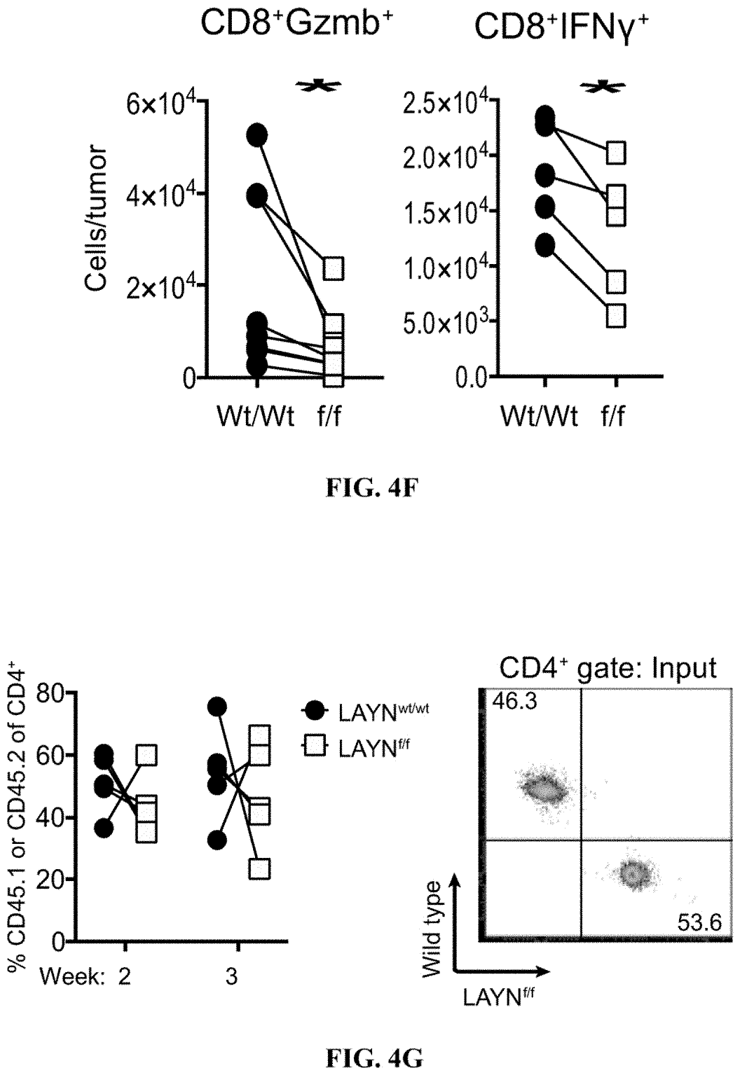

A competitive adoptive transfer approach was used to assess the cellular and molecular mechanisms by which layilin expression on CD8⁺ T cells attenuates tumor growth. Lymph node-derived CD8⁺ T cells from wildtype CD45.1⁺ and CD8^{Cre}Layn^{f/f} CD45.2⁺ mice were purified and transferred at 1:1 ratios into immunodeficient Rag2^{-/-} hosts. A schematic of the experimental design is illustrated in FIG. 4A. Two days later, mice were challenged subcutaneously with 5×10⁵ MC38-LUC2 tumor cells. The percentages of wild type (CD45.1) and LAYN^{-/-} (CD45.2) CD8⁺ T cells among tissue-infiltrating lymphocytes were quantified by flow cytometry. As shown in FIG. 4B, there was a marked reduction in the accumulation of layilin-deficient TILs when compared to layilin expressing controls at both 2- and 3-weeks (top panels are representative flow analyses for tumor samples; bottom three panel is quantification of individual mice at timepoints and tissues indicated). This data shows that layilin expression on CD8⁺ T cells conferred a selective advantage to accumulate in tumors

CD8+ TILs were also quantitatively phenotyped by flow cytometry. Paired comparison of wildtype and layilin-deficient CD8⁺ TILs revealed no cell intrinsic differences in granzyme B, IFNγ, or TNFα expression (FIG. 4C; left, middle, right panels, respectively). Similarly, PD-1 expression and proliferative capacity were unchanged between layilin-deficient and control TILs (FIGS. 4D and E, respectively). Increased accumulation of WT TILs (FIG. 4B) also resulted in a significant enrichment in the number of granzyme B and IFNγ producing CD8⁺ T cells in tumors when compared to layilin-deficient TILs (FIG. 4F). The difference in accumulation was not observed between co-injected CD4⁺ T cells from wildtype and CD8^{cre}Layn^{f/f} mice (FIG. 4G). Taken together, these results suggest that layilin does not significantly influence activation, proliferation or cytokine expression in CD8⁺ TILs, but instead predominantly enhances their accumulation in tumors.

### Example 5 Effect of Layilin on Beta Integrin Complex Activation

Methods and Materials

All human melanoma tumor samples were digested and prepared into single-cell suspensions as previously reported (R. S. Rodriguez et al., Memory regulatory T cells reside in human skin. J. Clin. Invest. 124, 1027-1036 (2014)). Briefly, samples were finely minced and digested for 12-14 hours at 37° C. in RPMI media containing 10% FBS, 1% HEPES, collagenase type IV (4188; Worthington Biochemical Corp.), DNase (SDN25-1G; Sigma-Aldrich), 10% FBS, 1% HEPES, and 1% penicillin-streptavidin. The resulting suspension was then filtered through a 100 μm sieve, washed, and pelleted in a 50 ml conical. The cells were then re-suspended and used for either multiparameter flow cytometry or FACS for bulk or single-cell RNA sequencing

Single-cell suspensions prepared as described above were stained with Ghost 510 Viability dye (Tonbo Biosciences) in PBS. Following a wash step, cells were stained for surface markers in PBS with 2% FCS. For intracellular staining, cells were fixed and permeabilized with the Foxp3/Transcription Factor Staining Buffer Set (eBiosciences, catalog 00-5523-00). For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

After the staining protocol described above, human single-cell suspensions from samples intended for RNA sequencing were sorted into TIL populations of interest using a FACSaria Fusion sorter (BD Biosciences). For the sort for the bulk RNA-seq comparing PD-1^{hi}CTLA-4^{hi} and PD-1^{lo}CLTA-4^{lo} CD8⁺ TILs, a small portion of each sample was set aside to serve as an intracellular staining control as only viable cells were sent for RNA sequencing which precluded the use of fixation and permeabilization. Intracellular staining controls included CTLA-4, and the PD-1 sorting gates were set based upon the CTLA-4 control gates so that >80% of sorted PD-1^{hi}CTLA-4^{hi} TILs had high levels of both markers. Viable CD45⁺ CD3⁺ CD8⁺ TILs were sorted for single-cell RNA-seq. For both bulk and single-cell RNA seq, cells were sorted into RPMI media containing 10% FBS and retained on ice. Samples for bulk RNA seq were pelleted and flash frozen prior in liquid nitrogen.

Fluorophore conjugated antibodies specific for mouse and human antigens were purchased from eBioscience, BD Biosciences, and Biolegend. The following clones were used for staining human cells: α-layilin (clone 3F7D7E2); α-CD8α (clone SK1); α-CD3 (clone SK7); α-CD18 (clone 1B4/CD18); α-Ki-67 (clone B56); α-PD-1 (EH12.2H7); α-LAG3 (3DS223H); α-TIGIT (MBSA43); α-CTLA-4 (14D3); α-granzyme B (clone GB11); α-IFNγ (4S.B3); and α-TNFα (MAb11). The α-layilin antibody was conjugated to biotin using the One-step Antibody Biotinylation Kit (Miltenyi Biotec, catalog no. 130-093-385) and detected with Streptavidin-Phycoerythrin (PE) (Biolegend). Antibodies for staining mouse cells: α-CD8α (clone 53-6.7); α-TCR-β (clone H57-597); α-CD4 (clone GK1.5); α-CD45.1 (clone A20); α-CD45.2 (clone 104); α-Ki67 (clone B56); α-IFNγ (clone XMG1.2); α-TNFα (clone MP6-XT22); α-granzyme B (clone GB11); α-PD-1 (clone 29F.1A12). EdU was detected using Click-iT™ flow cytometry kit (ThemoFisher Scientific).

Single-cell RNA-seq and TCR-seq libraries were prepared by the UCSF Core Immunology lab using the 10× Chromium Single Cell 5′ Gene Expression and V(D)J Profiling Solution kit, according to the manufacturer's instructions (10× Genomics, Pleasanton, Calif.). Briefly, individual cells were partitioned into barcoded Gel Beads-in emulsion (GEMs) with a mixture containing reverse transcriptase reagents. Incubation of the GEMs within a Chromium instrument resulted in 10× Barcoded and full-length cDNA that was thereafter purified and amplified with a thermal cycler. Amplified cDNA was then used to generate both a 5′ gene expression (GEX) library as well as a TCR library by using primers specific to the TCR constant regions. 150 paired-end sequencing was performed on a Novaseq 6000 instrument.

The Cell Ranger analysis pipelines (version 3.0.2, 10× Genomics) were then used to process the generated sequencing data. Data was demultiplexed into FASTQ files, aligned to the GRCh38 human reference genome and counted, and TCR library reads were assembled into single cell V(D)J sequences and annotations. For gene expression analysis, the R package Seurat (version 3.0) (Stuart, Butler, el al, biorxiv 2018) was used. Filtered gene-barcode matrices were loaded and quality-control steps were performed (low quality or dying cells and cell douplets/multiplets were excluded from subsequent analysis). Data was normalized and scaled, and then linear dimensional reduction with principle component analysis (PCA) was performed.

Proximity ligation assays were performed using the Duolink® PLA flow cytometry kit (Millipore Sigma) with the following antibodies: mouse α-layilin (clone 3F7D7E2; Sino Biological), rabbit α-CD18 (polyclonal; proteintech), and rabbit α-CD11a (clone EP1285Y; Abcam).

For measurement of LFA-1 activation, Jurkat E6-1 cells were transduced with a lentivirus (kind gift of Jeff Glasgow) containing a full length LAYN construct. Expressing cells were selected to form a stable line. LFA-1 activation was reported by staining the cells at 37° C. with clone m24 (Biolegend) in 20 mM HEPES; 140 mM NaCl; 1 mM MgCl₂; 1mMCaCl2; 2 mg/mL glucose; and 0.5% BSA. 2 mM MnCl₂ was used as a positive control, and 2 mM EDTA was added as a negative control.

Static adhesion experiments were performed by coating non-tissue culture treated polystyrene 96-well flat bottom plates with recombinant human ICAM-1 (R&D Systems) at 10 μg/mL. T cells were labeled with calcein AM (ThermoFisher Scientific) and loaded onto plates at 2×10⁶ cells/mL together with the indicated stimulus. PMA was added at 10 ng/mL while LFA-1 blocking was accomplished with 10 μg/mL anti-CD11a (clone HI111; ThermoFisher Scientific). After incubating for 15 minutes at 37° C., plates were flipped upside down and centrifuged at 50 g for 5 minutes. Fluorescence intensity was measured with a plate reader (PerkinElmer).

Results

The role of layilin on CD8⁺ T cells in enhancing cellular adhesion was explored. In addition, because layilin has a defined talin binding domain, the mechanism of layilin mediating its effects through modulation of talin binding integrins was explored.

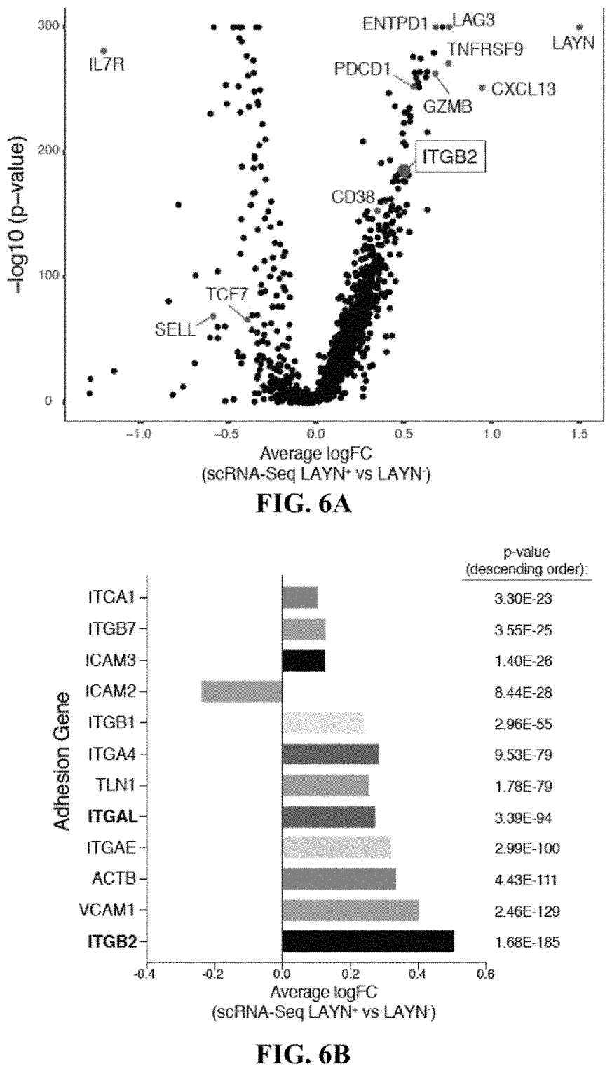

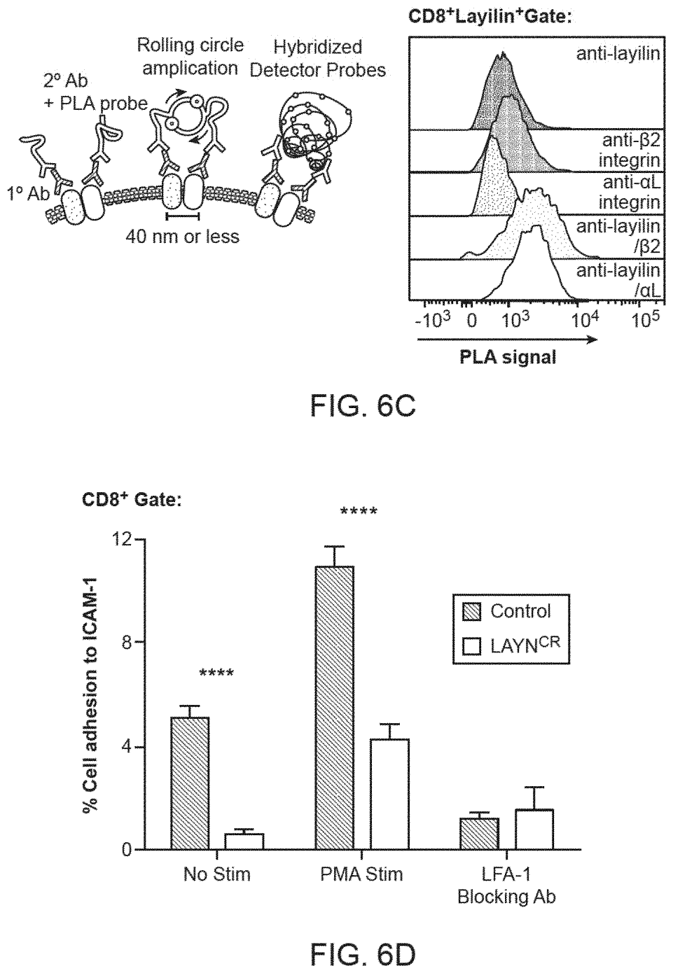

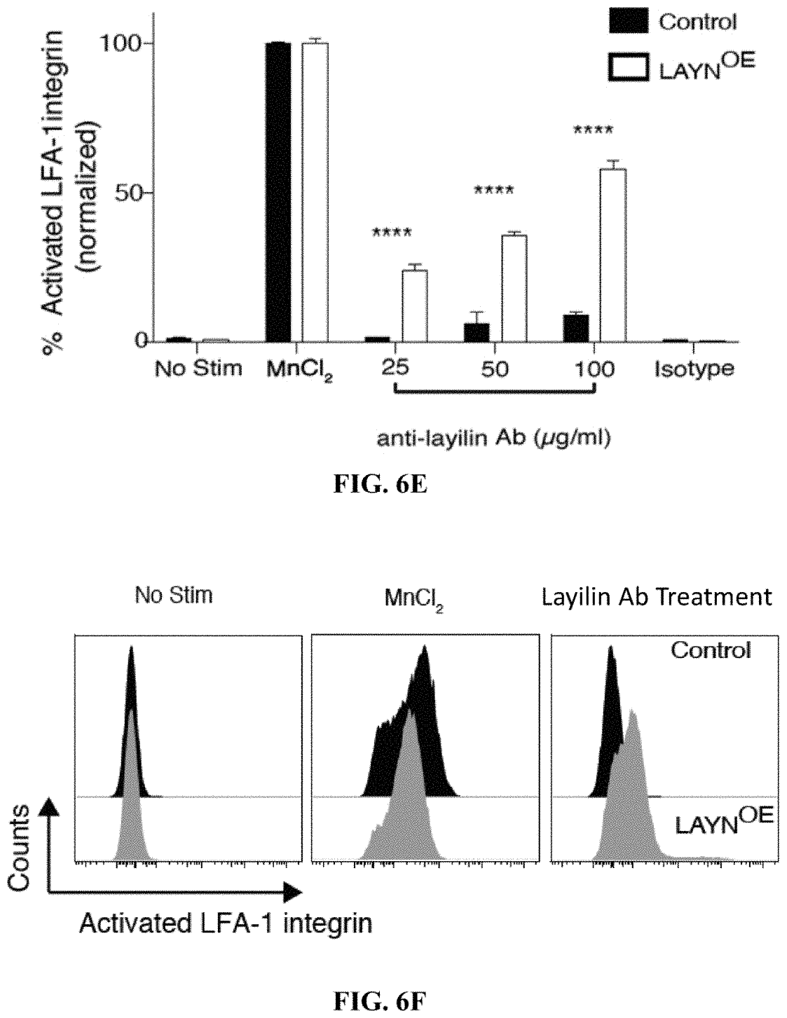

scRNA-seq data was analyzed to determine if genes involved in cellular adhesion were differentially expressed between LAYN⁺ and LAYN⁻ tumour infiltrating lymphocytes (TILs) isolated from patients with metastatic melanoma. Among genes enriched in LAYN⁺ TILs, ITGB2, which codes for integrin β2, separated out as one of the most differentially expressed genes (FIGS. 6A and 6B). While multiple integrin genes, including ITGB2's binding partner ITGAL, were significantly enriched in LAYN⁺ TILs, ITGB2 had the highest log fold change (p-value of 1.68×10-185) (FIG. 6B).

Integrins β2 and αL form the functional heterodimer, LFA-1, that is important in immune synapse formation and adhesion of cytotoxic T cells during killing of target cells (Anikeeva et al., 2005; Franciszkiewicz et al., 2013; Hammer et al., 2019). To determine if layilin is in close proximity and could potentially interact with LFA-1, a flow cytometric-based proximity ligation assay was performed. In this assay, a productive fluorescent signal is only observed if individual cell surface proteins are co-localized within 40 nm. Antibodies against αL, β2 or layilin alone generated minimal fluorescent signal. However, the combination of anti-layilin with anti-β2 or anti-αL antibodies generated a marked increase in fluorescent intensity (FIG. 6C). These data suggest that layilin co-localizes with LFA-1 on the surface of CD8⁺ T cells. Given this close association, whether layilin could influence LFA-1 activity in a static adhesion assay was functionally tested. Control and LAYN^{CR} CD8⁺ T cells were plated on ICAM-1 (the natural ligand for LFA-1) coated plates, and the number of cells remaining after centrifugal washing was quantified. Both in the presence and absence of T cell activation with phorbol 12-myristate 13-acetate, LAYN^{CR} cells displayed significantly reduced adhesion (FIG. 6D). Importantly, addition of LFA-1 blocking antibody (anti-CD11a clone HI111) abrogated all ICAM-1 binding, confirming that layilin-mediated enhancement of cell adhesion in this assay was dependent on LFA-1.

At steady-state LFA-1 integrin assumes a ‘closed’ low affinity confirmation and intracellular signaling or extracellular interactions induce a transformation to the ‘open’ high affinity form (Abram and Lowell, 2009; Sun et al., 2019). This conformational change is an important step in how LFA-1 mediates ligand binding and increased cell adhesion (Anikeeva et al., 2005; Franciszkiewicz et al., 2013). Whether the mechanism by which layilin enhances LFA-1-dependent adhesion is by enhancing the activation state of this integrin was explored. A Jurkat human T cell line was transduced with LAYN and the activated ‘open’ state of LFA-1 was quantified by flow cytometry. The m24 antibody that specifically recognizes the activated conformation of LFA-1 was used. While expression of layilin only minimally increased levels of activated LFA-1, a pronounced dose dependent increase in LFA-1 activation was observed upon addition of an anti-layilin monoclonal antibody (clone 3F7D7E2) (FIG. 6E and FIG. 6F). These data suggest that layilin enhances LFA-1 activation on T cells to augment cellular adhesion.

### Example 6—A Subset of Highly Activated TILs in Human Melanoma Express Layilin

Methods and Materials

All human melanoma tumor samples were digested and prepared into single-cell suspensions as previously reported (R. S. Rodriguez et al., Memory regulatory T cells reside in human skin. J. Clin. Invest. 124, 1027-1036 (2014)). Briefly, samples were finely minced and digested for 12-14 hours at 37° C. in RPMI media containing 10% FBS, 1% HEPES, collagenase type IV (4188; Worthington Biochemical Corp.), DNase (SDN25-1G; Sigma-Aldrich), 10% FBS, 1% HEPES, and 1% penicillin-streptavidin. The resulting suspension was then filtered through a 100 μm sieve, washed, and pelleted in a 50 ml conical. The cells were then re-suspended and used for either multiparameter flow cytometry or FACS for bulk or single-cell RNA sequencing

Single-cell suspensions prepared as described above were stained with Ghost 510 Viability dye (Tonbo Biosciences) in PBS. Following a wash step, cells were stained for surface markers in PBS with 2% FCS. For intracellular staining, cells were fixed and permeabilized with the Foxp3/Transcription Factor Staining Buffer Set (eBiosciences, catalog 00-5523-00). For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

After the staining protocol described above, human single-cell suspensions from samples intended for RNA sequencing were sorted into TIL populations of interest using a FACSaria Fusion sorter (BD Biosciences). For the sort for the bulk RNA-seq comparing PD-1^{hi}CTLA-4^{hi} and PD-1^{lo}CLTA-4^{lo} CD8⁺ TILs, a small portion of each sample was set aside to serve as an intracellular staining control as only viable cells were sent for RNA sequencing which precluded the use of fixation and permeabilization. Intracellular staining controls included CTLA-4, and the PD-1 sorting gates were set based upon the CTLA-4 control gates so that >80% of sorted PD-1^{hi}CTLA-4^{hi} TILs had high levels of both markers. Viable CD45⁺ CD3⁺ CD8⁺ TILs were sorted for single-cell RNA-seq. For both bulk and single-cell RNA seq, cells were sorted into RPMI media containing 10% FBS and retained on ice. Samples for bulk RNA seq were pelleted and flash frozen prior in liquid nitrogen.

Fluorophore conjugated antibodies specific for mouse and human antigens were purchased from eBioscience, BD Biosciences, and Biolegend. The following clones were used for staining human cells: α-layilin (clone 3F7D7E2); α-CD8α (clone SK1); α-CD3 (clone SK7); α-CD18 (clone 1B4/CD18); α-Ki-67 (clone B56); α-PD-1 (EH12.2H7); α-LAG3 (3DS223H); α-TIGIT (MBSA43); α-CTLA-4 (14D3); α-granzyme B (clone GB11); α-IFNγ (4S.B3); and α-TNFα (MAb11). The α-layilin antibody was conjugated to biotin using the One-step Antibody Biotinylation Kit (Miltenyi Biotec, catalog no. 130-093-385) and detected with Streptavidin-Phycoerythrin (PE) (Biolegend). Antibodies for staining mouse cells: α-CD8α (clone 53-6.7); α-TCR-β (clone H57-597); α-CD4 (clone GK1.5); α-CD45.1 (clone A20); α-CD45.2 (clone 104); α-Ki67 (clone B56); α-IFNγ (clone XMG1.2); α-TNFα (clone MP6-XT22); α-granzyme B (clone GB11); α-PD-1 (clone 29F.1A12). EdU was detected using Click-iT™ flow cytometry kit (ThemoFisher Scientific).

For bulk RNA sequencing, samples were sent as frozen cell pellets to Expression Analysis, Quintiles (Morrisville, N.C.) for all sample processing and sequencing steps. RNA isolation was performed with QIAGEN RNeasy Spin Columns, and RNA quality was assessed using an Agilent Bioanalyzer Pico Chip. RNA was then converted to complementary DNA (cDNA) libraries using the Illumina TruSeq Stranded mRNA sample preparation kit. Sequencing of cDNA libraries was performed to a 25 M read depth using an Illumina sequencing platform. After sequencing, TopHat (version 2.0.12) was used to align reads to the Ensembl GRCh38 reference genome, and SAMtools was used to generate SAM files. Htseq-count (0.6.1p1, with union option) was then used to generate read counts. Once the counts were obtained, differentially expressed genes between paired samples were determined using the R/Bioconductor package DESeq2.

Single-cell RNA-seq and TCR-seq libraries were prepared by the UCSF Core Immunology lab using the 10× Chromium Single Cell 5′ Gene Expression and V(D)J Profiling Solution kit, according to the manufacturer's instructions (10× Genomics, Pleasanton, Calif.). Briefly, individual cells were partitioned into barcoded Gel Beads-in emulsion (GEMs) with a mixture containing reverse transcriptase reagents. Incubation of the GEMs within a Chromium instrument resulted in 10× Barcoded and full-length cDNA that was thereafter purified and amplified with a thermal cycler. Amplified cDNA was then used to generate both a 5′ gene expression (GEX) library as well as a TCR library by using primers specific to the TCR constant regions. 150 paired-end sequencing was performed on a Novaseq 6000 instrument.

The Cell Ranger analysis pipelines (version 3.0.2, 10× Genomics) were then used to process the generated sequencing data. Data was demultiplexed into FASTQ files, aligned to the GRCh38 human reference genome and counted, and TCR library reads were assembled into single cell V(D)J sequences and annotations. For gene expression analysis, the R package Seurat (version 3.0) (cite Stuart, Butler, el al, biorxiv 2018) was used. Filtered gene-barcode matrices were loaded and quality-control steps were performed (low quality or dying cells and cell douplets/multiplets were excluded from subsequent analysis). Data was normalized and scaled, and then linear dimensional reduction with principle component analysis (PCA) was performed.

Results

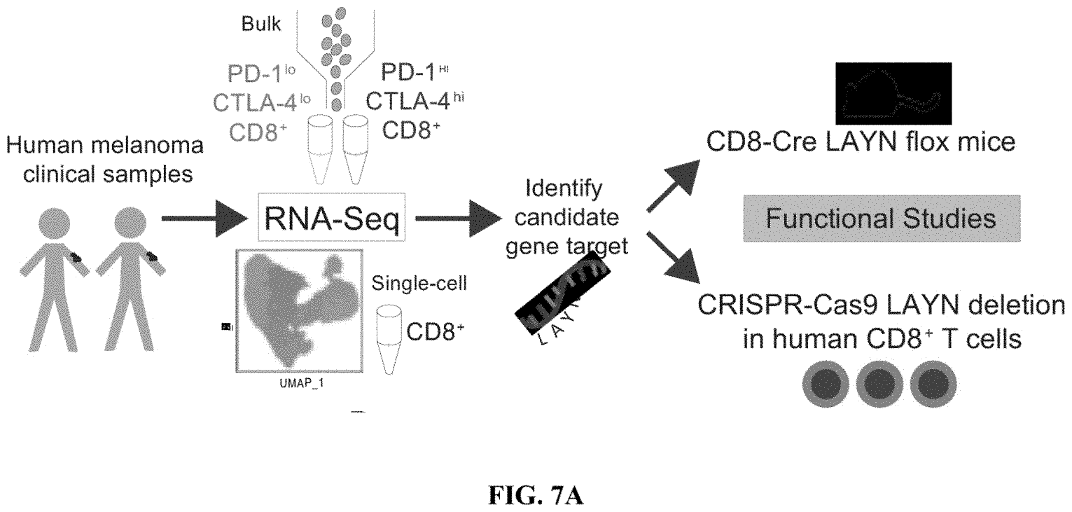

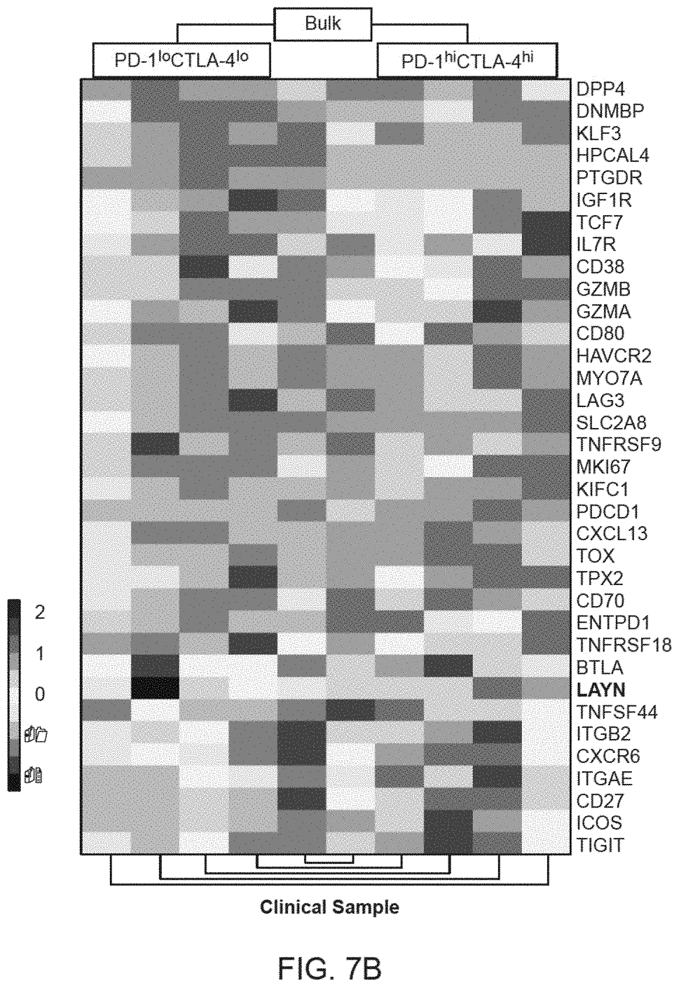

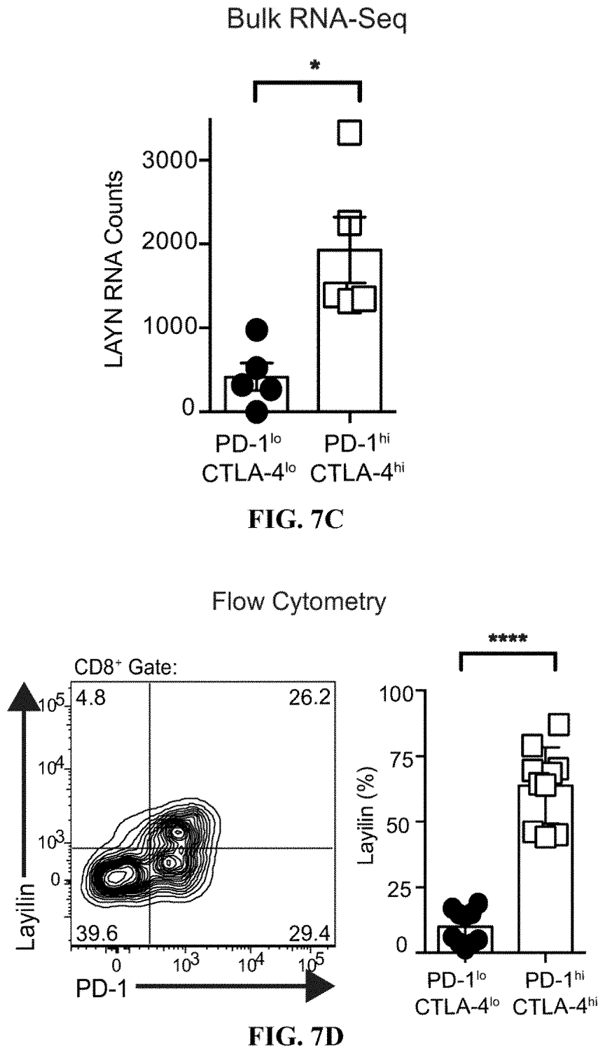

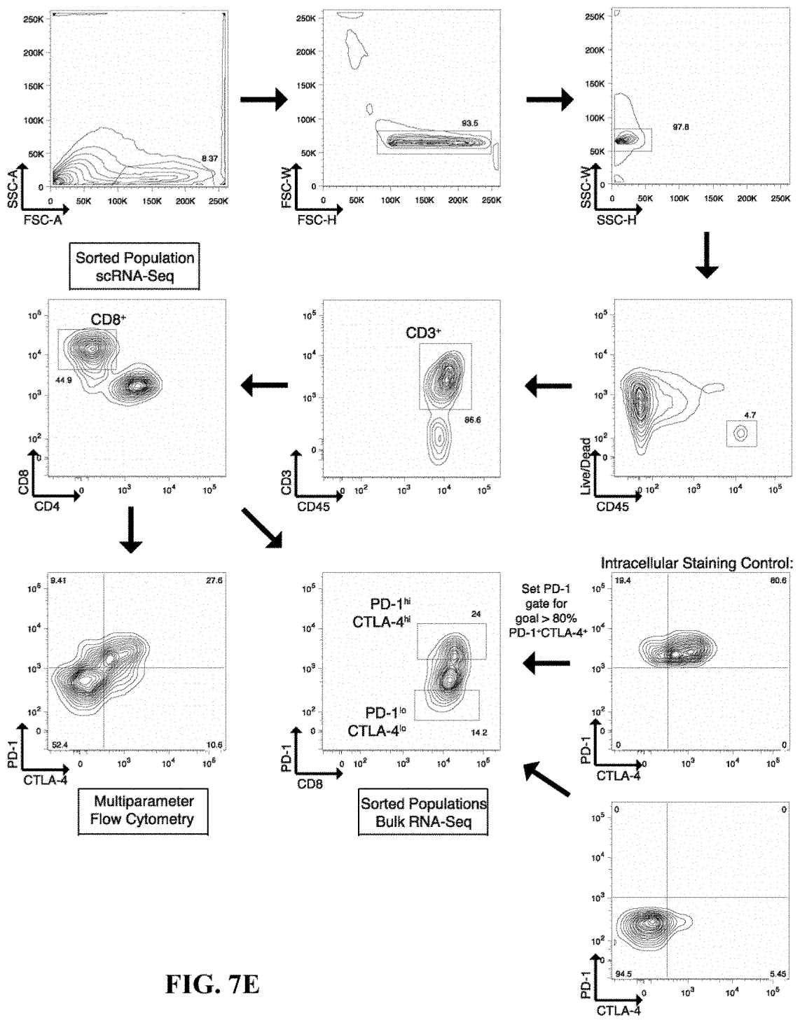

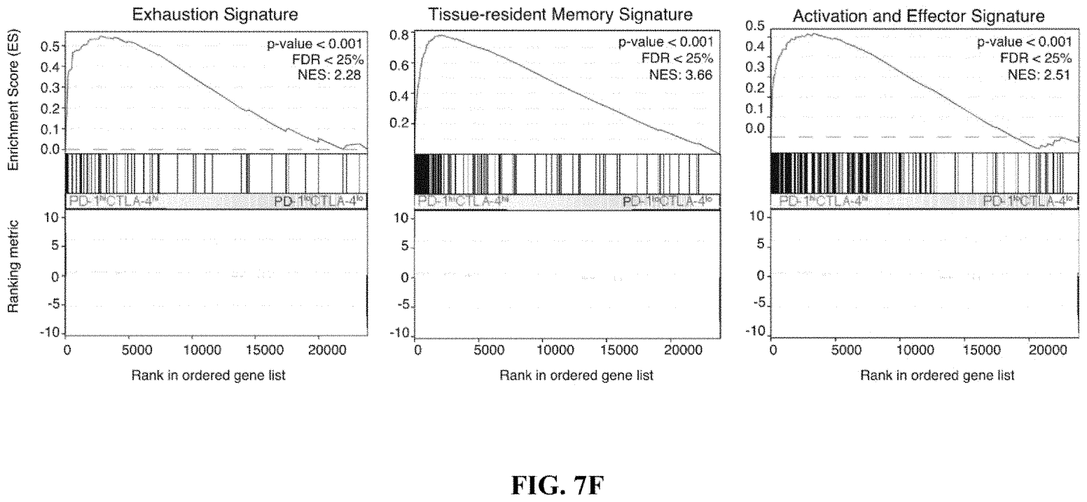

To understand the fundamental biology of PD-1^{hi}CTLA-4^{hi} CD8⁺ TILs, a fluorescence-activated cell sorting (FACS) strategy was used to isolate these cells from 8 melanoma patients and either bulk or single cell whole transcriptome RNA-sequencing (RNA-Seq) was performed, as schematized in FIG. 7A. The gating strategy used is shown in FIG. 7E. Table 1 presents the demographics of the donors. As expected, PD-1^{hi}CTLA-4^{hi} cells were enriched for expression of immune checkpoint receptors, activation markers and tissue resident memory genes (FIG. 7F). Differential expression analysis revealed the gene LAYN to be highly expressed in the PD-1^{hi}CTLA-4^{hi} TIL subset (FIG. 7B and FIG. 7C). LAYN codes for layilin, a C-type lectin domain containing cell surface glycoprotein (Borowsky and Hynes, 1998; Bono et al., 2001). Flow cytometric quantification of layilin validated its preferential expression on the cell surface of PD-1^{hi}CTLA-4^{hi} TILs in human metastatic melanoma (FIG. 7D).

TABLE 1
Clinical sample and human donor demographics

| Donor ID | Age | Gender | Site | Treatment Status | Study |
|---|---|---|---|---|---|
| K-254 | 74 | Male | Extremity | Naïve | Bulk RNA-seq |
| K-288 | 46 | Male | Axillary LN | Naïve | Bulk RNA-seq |
| K-312 | 61 | Male | Axillary LN | Naïve | Bulk RNA-seq |
| K-314 | 60 | Female | Axillary LN | Naïve | Bulk RNA-seq |
| K-315 | 67 | Male | Inguinal LN | Naïve | Bulk RNA-seq |
| K-383 | 45 | Male | Inguinal LN | Naïve | scRNA-seq |
| K-404 | 52 | Female | Mediastinal LN | Gamma Knife | Flow cytometry |
| K-406 | 76 | Female | Extremity | Naïve | Flow cytometry |
| K-409 | 65 | Male | Extremity, | Naive | scRNA-seq, Flow |
| | | | Inguinal LN, | | cytometry |
| | | | Blood | | |
| K-411 | 58 | Female | Neck LN | Naïve | scRNA-seq |
| K-414 | 88 | Male | Chest | Naive | Flow cytometry |
| K-427 | 60 | Male | Gluteus | Naive | Flow cytometry |
| K-447 | 66 | Male | Chest | Naïve | Flow cytometry |
| K-458 | 62 | Male | Extremity | Naive | Flow cytometry |
| K-459 | 62 | Female | Trunk | Trametinib/Nivolumab | Flow cytometry |
| K-479 | 71 | Male | Neck LN | Naive | Flow cytometry |
| K-483 | 70 | Male | Axillary LN | Naive | Flow cytometry |
| 11197 | 24 | Male | PBMCs | N/A: healthy | In vitro CRISPR |
| 12009 | 21 | Male | PBMCs | N/A: healthy | In vitro CRISPR |

### Example 7 Highly Activated, Clonally Expanded CD8⁺ TILs Specifically Upregulate Layilin

Methods and Materials

All human melanoma tumor samples were digested and prepared into single-cell suspensions as previously reported (R. S. Rodriguez et al., Memory regulatory T cells reside in human skin. J. Clin. Invest. 124, 1027-1036 (2014)). Briefly, samples were finely minced and digested for 12-14 hours at 37° C. in RPMI media containing 10% FBS, 1% HEPES, collagenase type IV (4188; Worthington Biochemical Corp.), DNase (SDN25-1G; Sigma-Aldrich), 10% FBS, 1% HEPES, and 1% penicillin-streptavidin. The resulting suspension was then filtered through a 100 μm sieve, washed, and pelleted in a 50 ml conical. The cells were then re-suspended and used for either multiparameter flow cytometry or FACS for bulk or single-cell RNA sequencing

Single-cell suspensions prepared as described above were stained with Ghost 510 Viability dye (Tonbo Biosciences) in PBS. Following a wash step, cells were stained for surface markers in PBS with 2% FCS. For intracellular staining, cells were fixed and permeabilized with the Foxp3/Transcription Factor Staining Buffer Set (eBiosciences, catalog 00-5523-00). For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

After the staining protocol described above, human single-cell suspensions from samples intended for RNA sequencing were sorted into TIL populations of interest using a FACSaria Fusion sorter (BD Biosciences). For the sort for the bulk RNA-seq comparing PD-1^{hi}CTLA-4^{hi} and PD-1^{lo}CLTA-4^{lo} CD8⁺ TILs, a small portion of each sample was set aside to serve as an intracellular staining control as only viable cells were sent for RNA sequencing which precluded the use of fixation and permeabilization. Intracellular staining controls included CTLA-4, and the PD-1 sorting gates were set based upon the CTLA-4 control gates so that >80% of sorted PD-1^{hi}CTLA-4^{hi} TILs had high levels of both markers. Viable CD45⁺ CD3⁺ CD8⁺ TILs were sorted for single-cell RNA-seq. For both bulk and single-cell RNA seq, cells were sorted into RPMI media containing 10% FBS and retained on ice. Samples for bulk RNA seq were pelleted and flash frozen prior in liquid nitrogen.

Fluorophore conjugated antibodies specific for mouse and human antigens were purchased from eBioscience, BD Biosciences, and Biolegend. The following clones were used for staining human cells: α-layilin (clone 3F7D7E2); α-CD8α (clone SK1); α-CD3 (clone SK7); α-CD18 (clone 1B4/CD18); α-Ki-67 (clone B56); α-PD-1 (EH12.2H7); α-LAG3 (3DS223H); α-TIGIT (MBSA43); α-CTLA-4 (14D3); α-granzyme B (clone GB11); α-IFNγ (4S.B3); and α-TNFα (MAb11). The α-layilin antibody was conjugated to biotin using the One-step Antibody Biotinylation Kit (Miltenyi Biotec, catalog no. 130-093-385) and detected with Streptavidin-Phycoerythrin (PE) (Biolegend). Antibodies for staining mouse cells: α-CD8α (clone 53-6.7); α-TCR-β (clone H57-597); α-CD4 (clone GK1.5); α-CD45.1 (clone A20); α-CD45.2 (clone 104); α-Ki67 (clone B56); α-IFNγ (clone XMG1.2); α-TNFα (clone MP6-XT22); α-granzyme B (clone GB11); α-PD-1 (clone 29F.1A12). EdU was detected using Click-iT™ flow cytometry kit (ThemoFisher Scientific).

For bulk RNA sequencing, samples were sent as frozen cell pellets to Expression Analysis, Quintiles (Morrisville, N.C.) for all sample processing and sequencing steps. RNA isolation was performed with QIAGEN RNeasy Spin Columns, and RNA quality was assessed using an Agilent Bioanalyzer Pico Chip. RNA was then converted to complementary DNA (cDNA) libraries using the Illumina TruSeq Stranded mRNA sample preparation kit. Sequencing of cDNA libraries was performed to a 25 M read depth using an Illumina sequencing platform. After sequencing, TopHat (version 2.0.12) was used to align reads to the Ensembl GRCh38 reference genome, and SAMtools was used to generate SAM files. Htseq-count (0.6.1p1, with union option) was then used to generate read counts. Once the counts were obtained, differentially expressed genes between paired samples were determined using the R/Bioconductor package DESeq2.

Single-cell RNA-seq and TCR-seq libraries were prepared by the UCSF Core Immunology lab using the 10× Chromium Single Cell 5′ Gene Expression and V(D)J Profiling Solution kit, according to the manufacturer's instructions (10× Genomics, Pleasanton, Calif.). Briefly, individual cells were partitioned into barcoded Gel Beads-in emulsion (GEMs) with a mixture containing reverse transcriptase reagents. Incubation of the GEMs within a Chromium instrument resulted in 10× Barcoded and full-length cDNA that was thereafter purified and amplified with a thermal cycler. Amplified cDNA was then used to generate both a 5′ gene expression (GEX) library as well as a TCR library by using primers specific to the TCR constant regions. 150 paired-end sequencing was performed on a Novaseq 6000 instrument.

The Cell Ranger analysis pipelines (version 3.0.2, 10× Genomics) were then used to process the generated sequencing data. Data was demultiplexed into FASTQ files, aligned to the GRCh38 human reference genome and counted, and TCR library reads were assembled into single cell V(D)J sequences and annotations. For gene expression analysis, the R package Seurat (version 3.0) (cite Stuart, Butler, el al, biorxiv 2018) was used. Filtered gene-barcode matrices were loaded and quality-control steps were performed (low quality or dying cells and cell douplets/multiplets were excluded from subsequent analysis). Data was normalized and scaled, and then linear dimensional reduction with principle component analysis (PCA) was performed.

UMAP visualizations were generated with the CATALYST package (Nowicka et al., 2017) (1.10.1) using CD8+ cells (CD45+CD3+CD4−CD8+) exported manually from biaxial plots in FlowJo (10.6.1).

Results

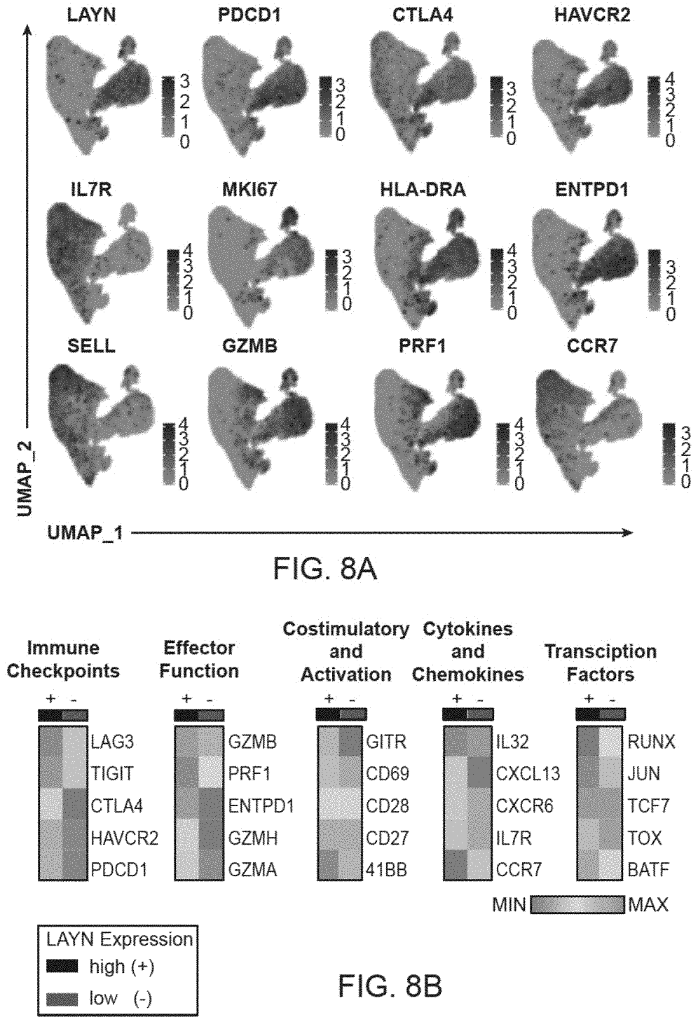

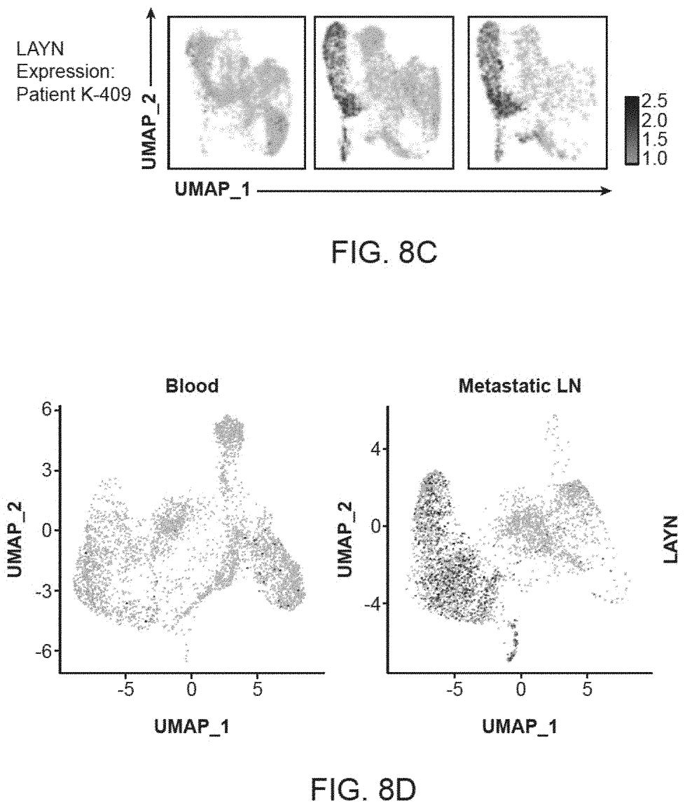

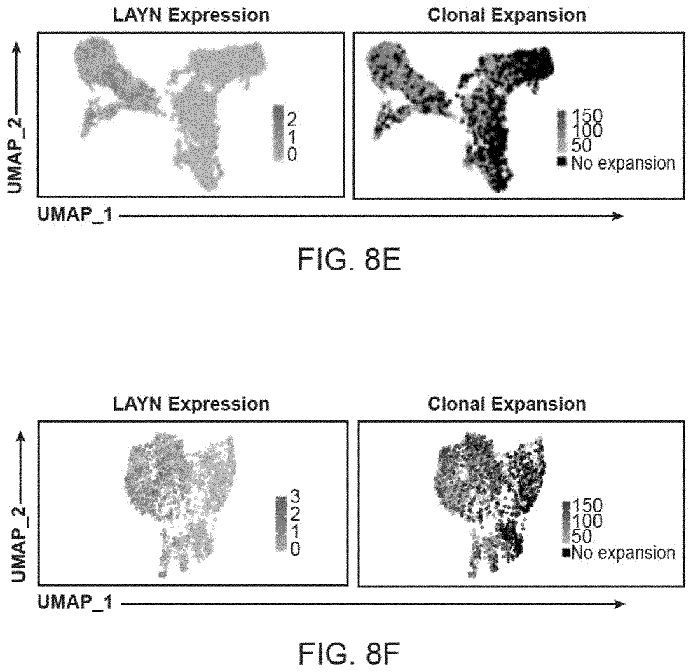

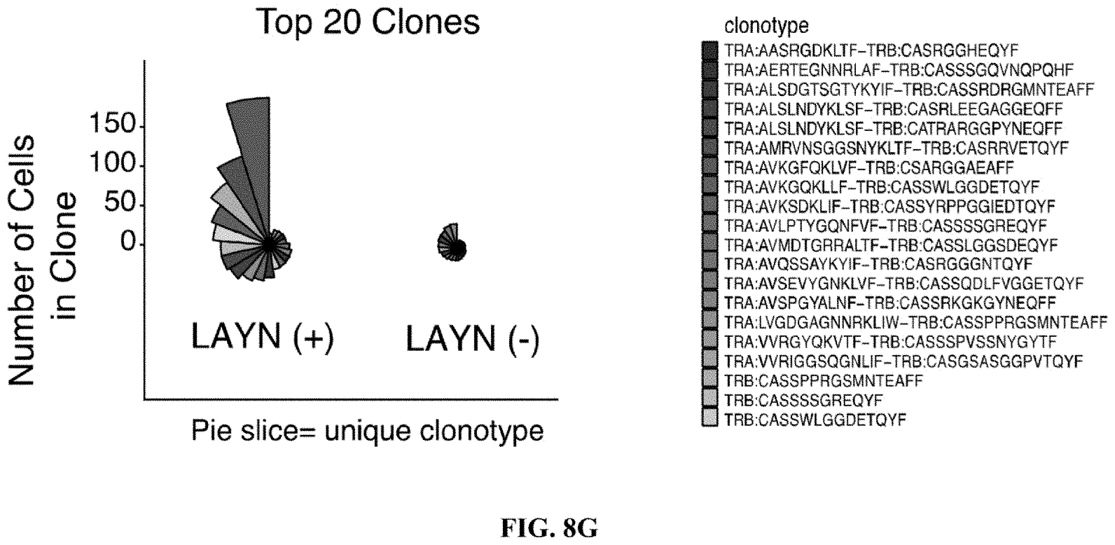

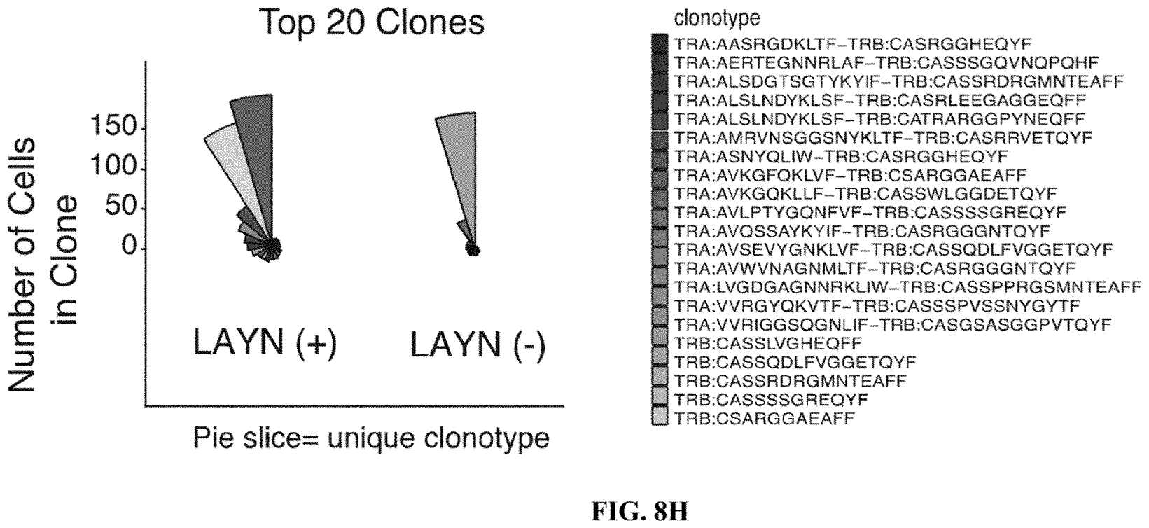

Single cell RNA-seq (scRNA-seq) was performed on 20,018 CD3⁺ CD8⁺ T cells freshly isolated from metastatic melanoma tumors. Unbiased clustering was performed and clusters were visualized with Uniform Maniford Approximation and Project (UMAP) dimensional reduction. LAYN closely overlapped with inhibitory receptors, activation and effector molecules, as well as tissue resident memory genes (FIGS. 8A and 8B). In contrast, LAYN expressing cells were distinct from IL-7R, L-selectin (SELL) and CCR7 expressing cells, further suggesting a tissue resident phenotype. To determine if LAYN expressing TILs are primarily found in tumors, scRNA-seq was performed on CD8⁺ T cells isolated from the peripheral blood, involved lymph node (LN) and primary tumor in a patient with stage III melanoma. LAYN was highly expressed in both the tumor and involved lymph node, but nearly absent in peripheral blood (FIGS. 8C and 8D). T cell receptor (TCR) sequence analysis revealed that LAYN expression in both primary tumor and involved LNs closely overlapped with expanded CD8⁺ T cell clones (FIG. 8E involved lymph node; FIG. 8F—primary tumor). Notably, the top 20 expanded clonotypes (which represented the majority of all cells sequenced) were primarily found in the LAYN expressing cells (FIG. 8G—involved lymph node; FIG. 8H primary tumor). Additionally, presence of the extracellular ATPase CD39, which identifies TILs recognizing tumor antigens, closely correlated with layilin expression (Yost et al., 2019; Simoni et al., 2018; Duhen et al., 2018) (FIG. 8I). Taken together, these results suggest that layilin is selectively expressed on a clonally expanded, and likely tumor specific, subset of tumor-resident CD8⁺ T cells in human melanoma.

### Example 8 Layilin Expression on CD8⁺ T Cells Enhances Tumor Cell Killing

Methods and Materials

Human PBMCs from two individual donors were purchased from AllCells (Alameda, Calif.). CD8⁺ T cells were enriched from these samples using a negative selection kit (STEMCELL Technologies). Isolated T cells were activated with αCD3/CD28 ImmunoCult™ reagent and grown in ImmunoCult™-XF T cell Expansion Medium (STEMCELL Technologies) with the addition of 10 ng/mL IL-15 and 100 U/mL IL-2. To delete LAYN at the genomic level, a guide RNA targeting exon 4 (sgRNA target sequence GGTCATGTACCATCAGCCAT (SEQ ID NO: 9)) and a non-targeting “scramble” control sequence (GGTTCTTGACTACCGTAAT (SEQ ID NO: 10)); guide RNAs were purchased from Integrated DNA Technologies (Iowa, Calif.). Recombinant Cas9 protein (UC Berkeley QB3 Macrolab, CA) was combined with guide RNA and introduced into primary T cells via electroporation as previously described. Cells were subsequently cultured for four days before analyzing or incorporating into functional assays.

Cytotoxicity assays were designed as previously described. Briefly, CD8⁺ T cells were transduced with lentivirus (kind gift of Jeff Glasgow) containing the 1G4 NY-ES01 reactive a95:LY TCR construct and sort-purified to generate a uniform population. These cells then underwent LAYN deletion with CRISPR-Cas9 gene editing (described above) and were cocultured with A375 melanoma cells expressing RFP in varying cellular ratios. A375 numbers were monitored over 5 days using the IncuCyte platform (Sartorius, Germany). A375 melanoma-T cell co-culture supernatants were collected on day five and measured for IFNγ and TNFα secretion by multiplex ELISA (Eve Technologies).

Single-cell suspensions were stained with Ghost 510 Viability dye (Tonbo Biosciences) in PBS. Following a wash step, cells were stained for surface markers in PBS with 2% FCS. For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

Fluorophore conjugated antibodies specific for mouse and human antigens were purchased from eBioscience, BD Biosciences, and Biolegend. The following clones were used for staining human cells: α-layilin (clone 3F7D7E2); α-CD8α (clone SK1); α-CD3 (clone SK7); α-CD18 (clone 1B4/CD18); α-Ki-67 (clone B56); α-PD-1 (EH12.2H7); α-LAG3 (3DS223H); α-TIGIT (MBSA43); α-CTLA-4 (14D3); α-granzyme B (clone GB11); α-IFNγ (4S.B3); and α-TNFα (MAb11). The α-layilin antibody was conjugated to biotin using the One-step Antibody Biotinylation Kit (Miltenyi Biotec, catalog no. 130-093-385) and detected with Streptavidin-Phycoerythrin (PE) (Biolegend).

Results

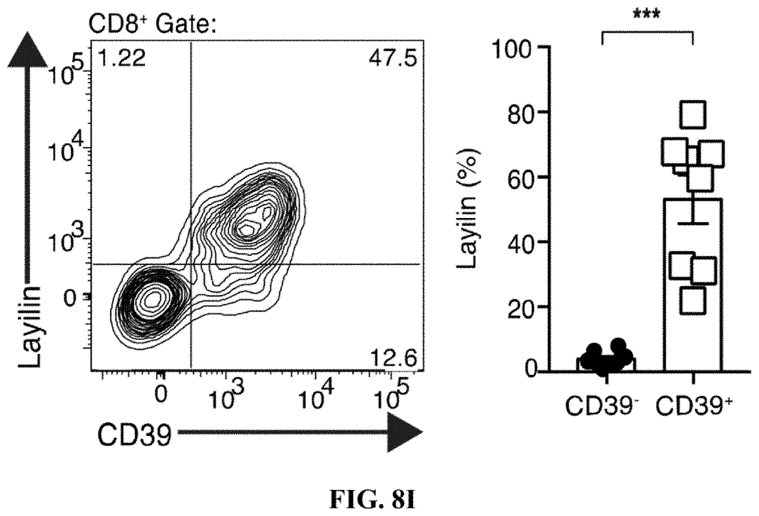

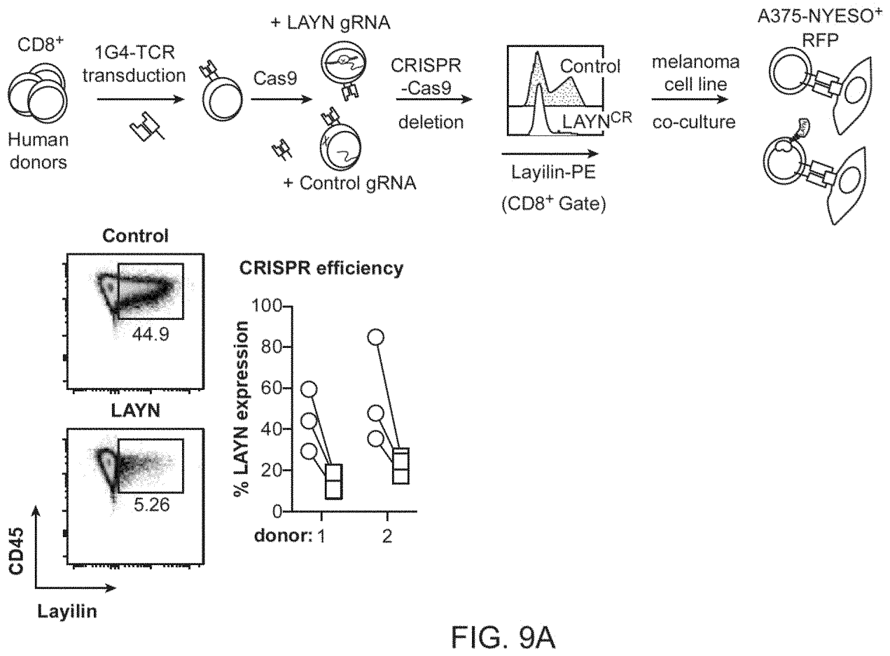

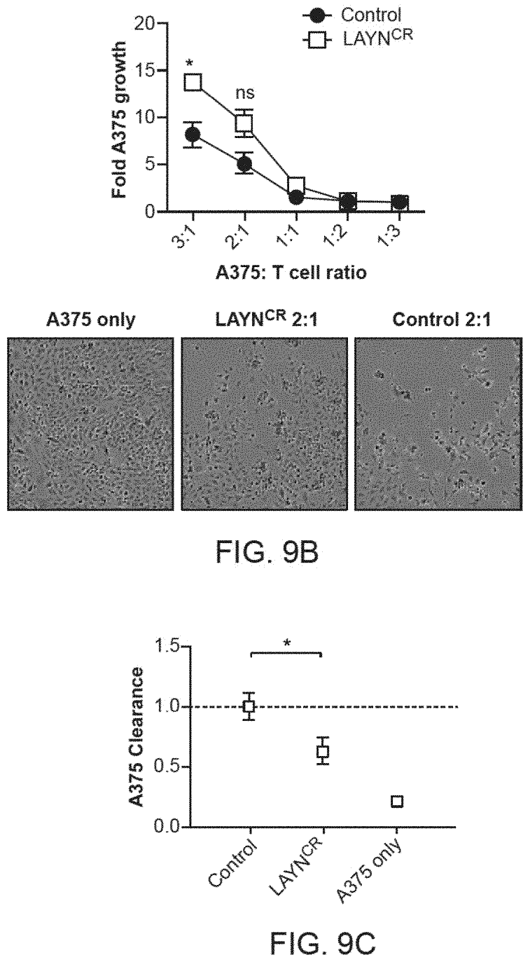

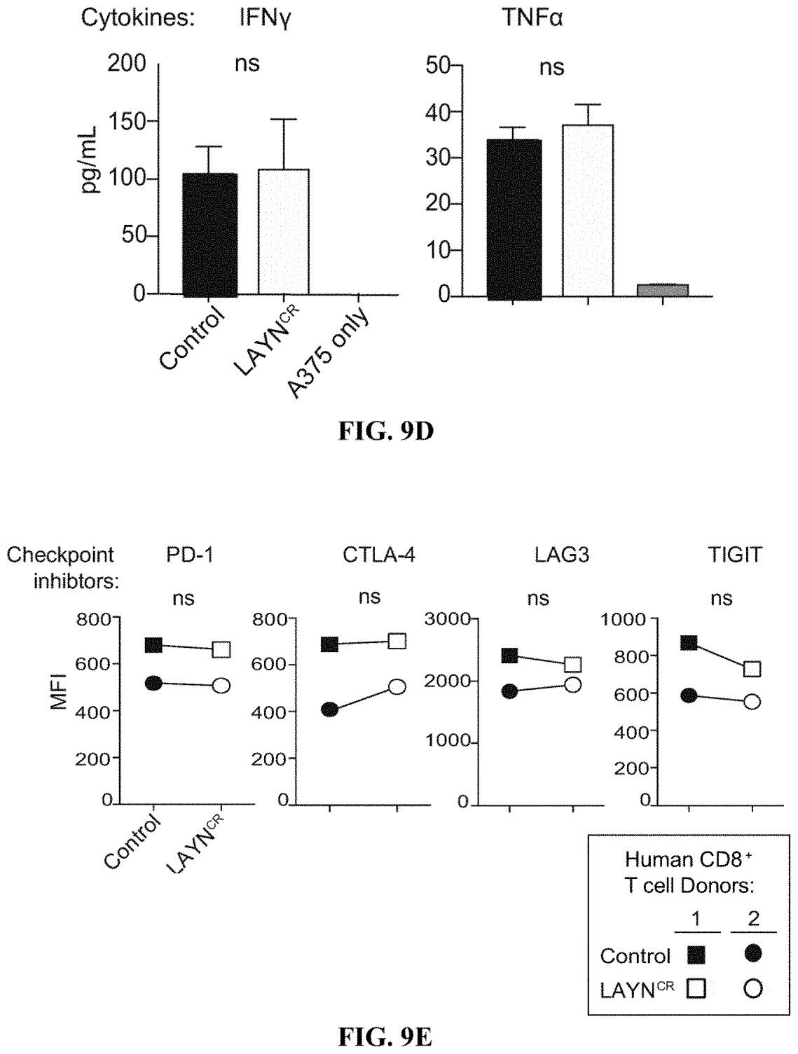

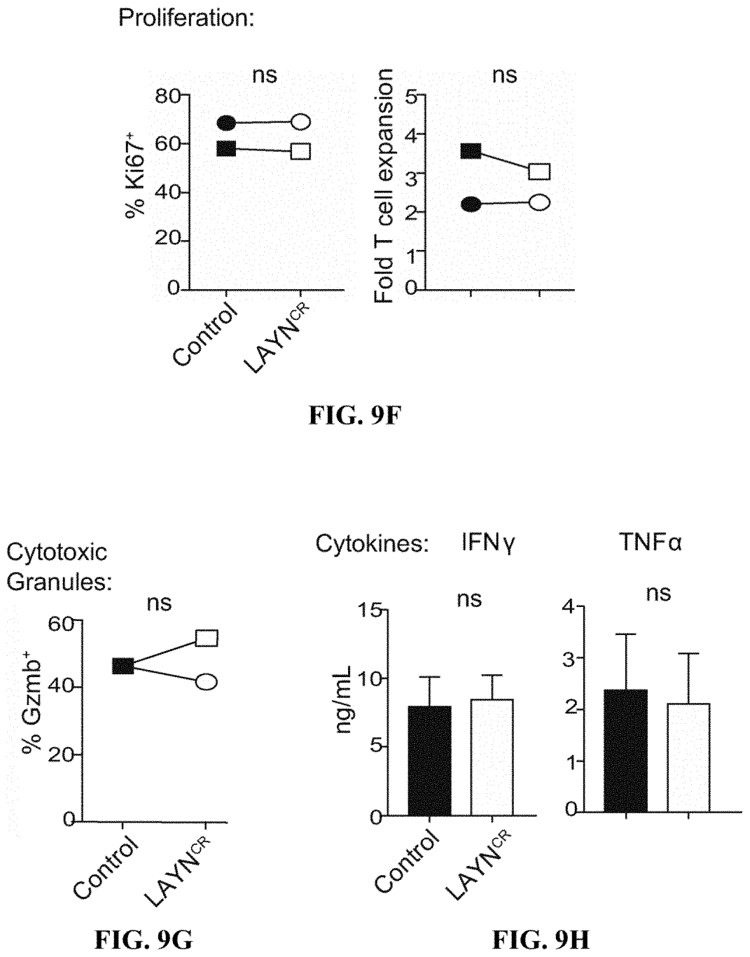

A CRISPR-Cas9 gene editing approach, as schematized in FIG. 9A (top panel), to disrupt LAYN in primary human CD8⁺ T cells was established to further assess the in vivo role and mechanism of layilin. Electroporative delivery of Cas9 pre-loaded with single guide RNA (sgRNA) (Schumann et al., 2015; Roth et al., 2018) targeting the LAYN gene significantly reduced layilin protein expression when compared to non-targeted control gRNA (FIG. 9A, bottom panel). To test whether layilin expression on CD8⁺ T cells plays a role in direct tumor cell killing, a well-established ex vivo antigen-specific tumor cytolytic model (Shifrut et al., 2018) was used, as further schematized in FIG. 9A (top panel). Briefly, purified CD8⁺ T cells were transduced to express the 1G4 TCR specific for the NY-ESO tumor antigen, and LAYN was subsequently deleted in these cells using our CRISPR-Cas9 approach (LAYN^{CR}). The LAYN^{CR} cells, or cells electroporated with a control gRNA, were co-cultured with A375-NY-ESO⁺ melanoma cancer cells and quantified A375 cell accumulation over five days. Consistent with mouse experiments, layilin deficient human CD8⁺ T cells were significantly less effective at killing tumor cells, especially at higher target to T cell ratios (FIGS. 9B and 9C). These results suggest that, in addition to promoting accumulation in tumors, layilin expression on CD8⁺ T cells plays a direct role in tumor cell killing.

To assess how layilin expression affects the function of CD8⁺ T cells, cytokines in the supernatants of the tumor/antigen-specific T cell cocultures (1G4-TCR⁺ CD8⁺ T cells with A375-NY-ES0⁺ melanoma cells) were examined. This analysis revealed similar levels of IFNγ and TNFα between control and LAYN^{CR} cultures (FIG. 9D). LAYN^{CR} cells activated by anti-CD3/CD28 stimulation were comprehensively phenotyped. When compared to control CD8⁺ T cells treated with non-targeted gRNA, no discernable differences were observed in the expression of the inhibitory receptors PD-1, CTLA-4, LAG3, and TIGIT (FIG. 9E). Furthermore, there was no difference in T cell proliferation, as measured by Ki67 expression and cumulative T cell expansion (FIG. 9F). Expression of the cytolytic protease granzyme B remained unchanged (FIG. 9G). In agreement with our tumor coculture experiments, there was no difference in the secretion of effector cytokines IFNγ and TNFα between layilin deleted and control cells (FIG. 9H). Consistent with the in vivo studies in mice described herein, these results indicate that layilin expression on CD8⁺ T cells does not influence proinflammatory cytokine secretion, cytolytic protein expression, cellular proliferation or inhibitory receptor expression in vitro.

### Example 9 Inhibition of Layilin in Hidradenitis Suppurativa Skin Explant Model

Methods and Materials

Skin biopsies were acquired from a male 50 year old buttocks diagnosed with Hidradenitis Suppurativa by dermatome and subjected to overnight digestion at 37 C in 250 U/mL Collagenase Type 4, 0.02 mg/ml DNAse, 10% fetal bovine serum (FBS), 100 uM HEPES, 1% penicillin/streptomycin, and 1% Glutamine in RPMI-1640 medium. Dissociated cells were washed and resuspended in X-Vivo 15 supplemented with 10% FBS, 1% non-essential amino acids, 1% sodium pyruvate and 1% penicillin/streptomycin. Samples were activated by plate-immobilized anti-CD3 and anti-CD28 at 0.1 ug/mL with or without 50 ug/mL anti-Layilin clone 3F7D7E2. After 2 days, samples were collected from culture and analysed by flow cytometry. Following a wash step, cells were stained for surface markers in PBS with 2% FCS. For multiparameter flow cytometry, samples were run on a LSRFortessa analyzer (355; 405; 488; 532; 561; 640 laser configuration; BD Biosciences) in the UCSF flow cytometry core and collected using FACS Diva software (BD Biosciences). Compensation was performed using UltraComp eBeads as single color controls (ThermoFisher Scientific). Data was analyzed using FlowJo software (Tree Star Inc.).

Fluorophore conjugated antibodies specific for human antigens were purchased from eBioscience, BD Biosciences, and Biolegend. The following clones were used for staining human cells: α-layilin (clone 3F7D7E2); α-CD8α (clone SK1); α-CD3 (clone SK7); α-granzyme B (clone GB11). The α-layilin antibody was conjugated to biotin using the One-step Antibody Biotinylation Kit (Miltenyi Biotec, catalog no. 130-093-385) and detected with Streptavidin-Phycoerythrin (PE) (Biolegend).

Results

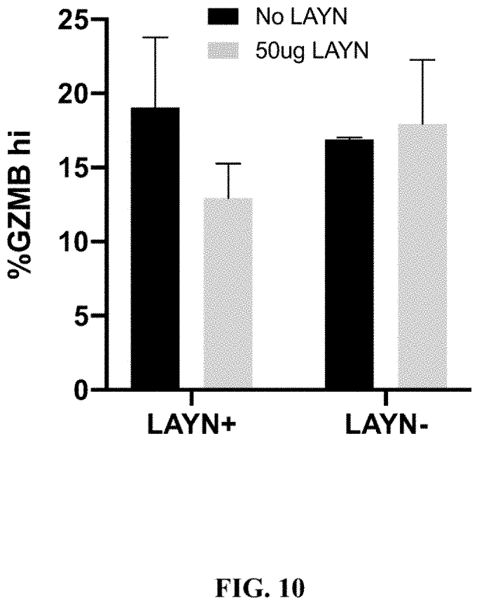

A skin explant was performed to assess the ability of an anti-layilin antibody to alter CD8 T cell function. As shown in FIG. 10, skin explants treated with the anti-layilin antibody (right column) demonstrated reduced granzyme-B based off myeloid cells (which do not express granzyme-B) as internal negative controls, in comparison to untreated skin explants (left column). Notably, the reduced granzyme-B was observed in LAYN⁺, but not LAYN⁻, CD8 T cells. The results suggests use of an anti-layilin antibody can reduce the inflammatory phenotype of LAYN+CD8 T cells in the context of disease.

### Example 10—a Subset of Highly Activated Tregs Express Layilin in Healthy and Diseased Human Skin

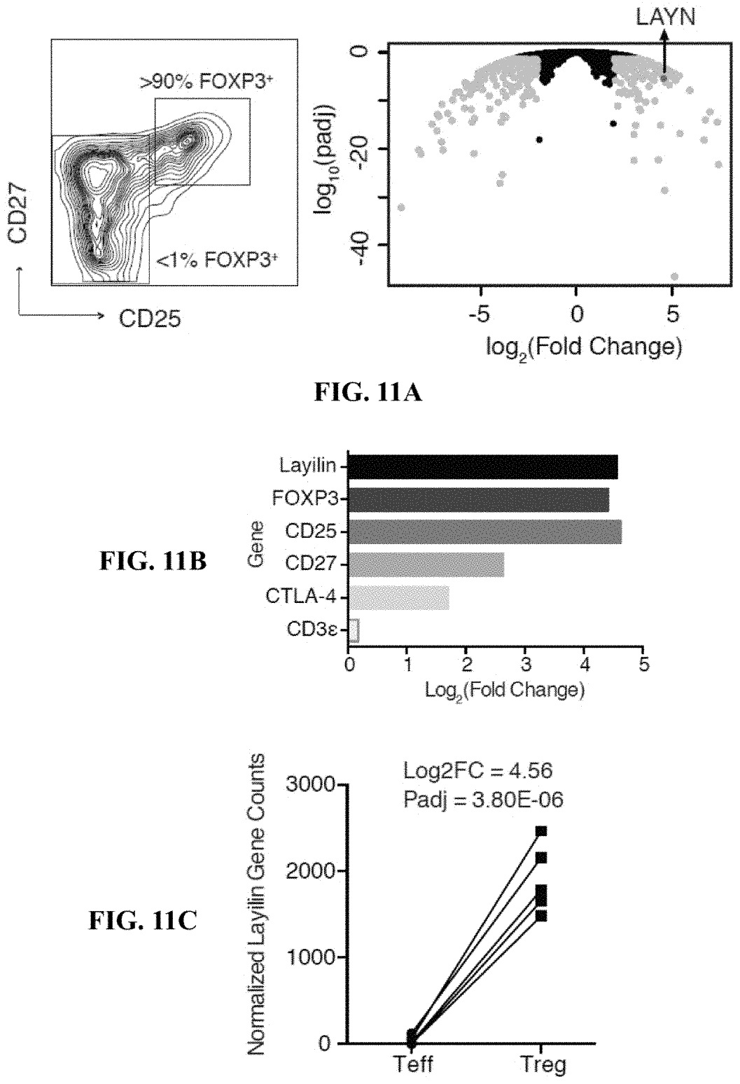

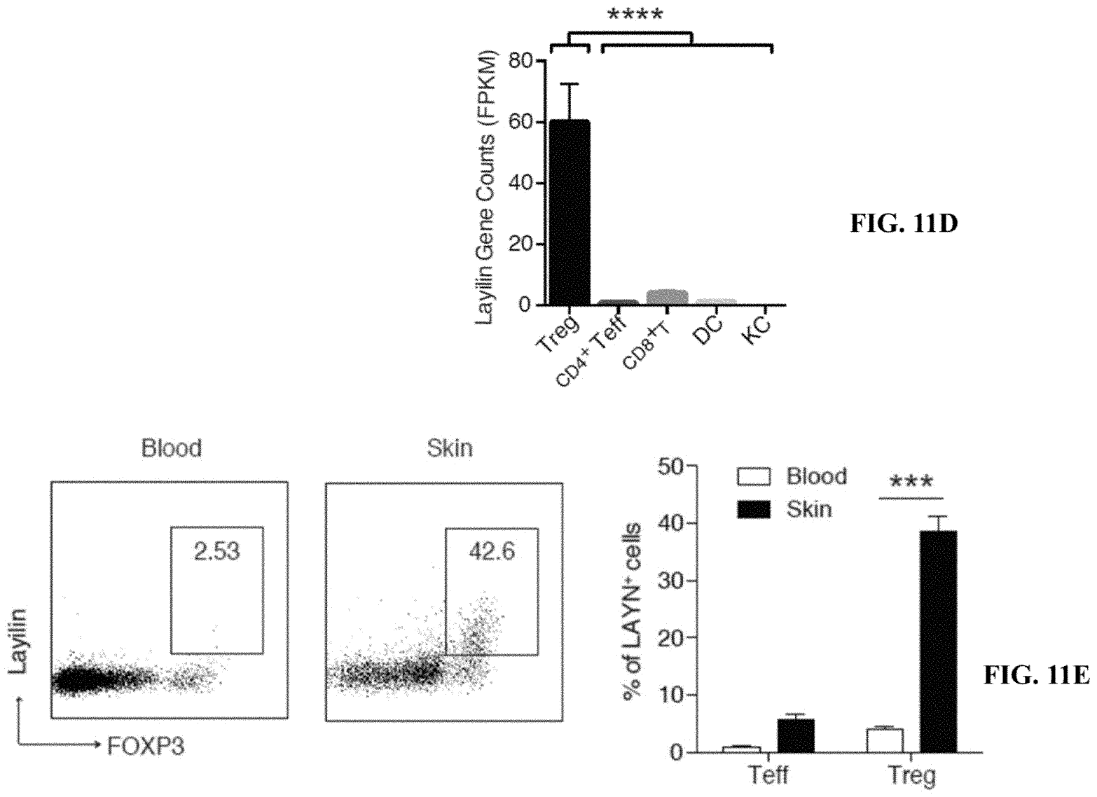

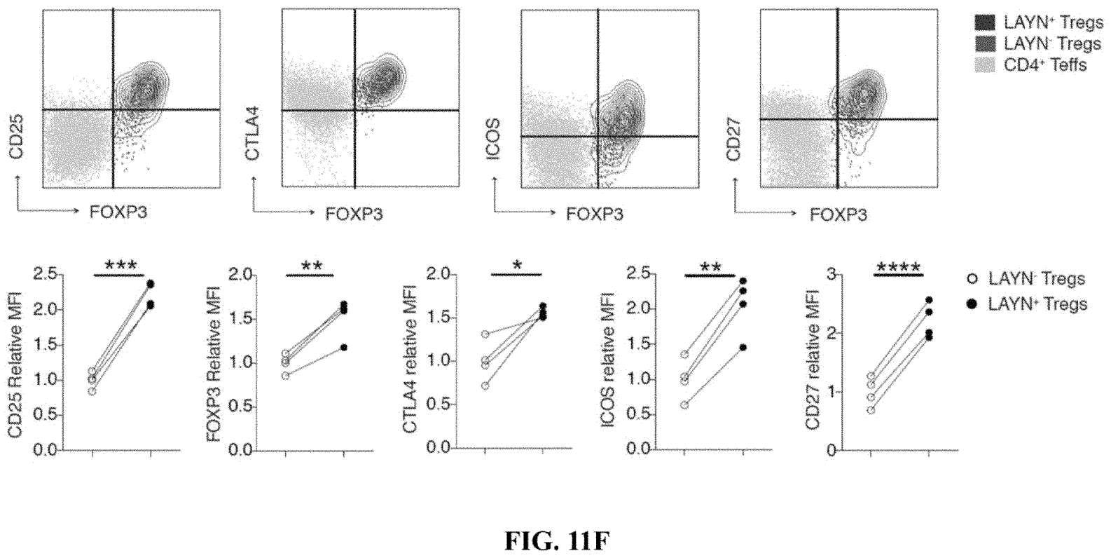

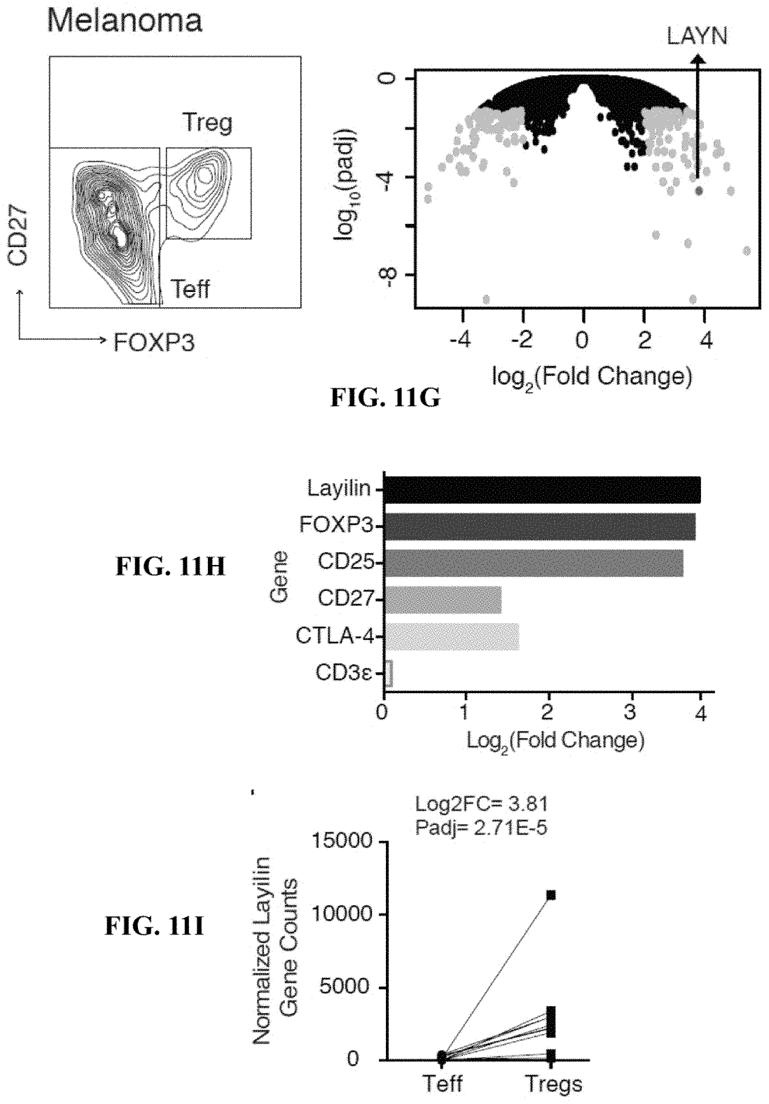

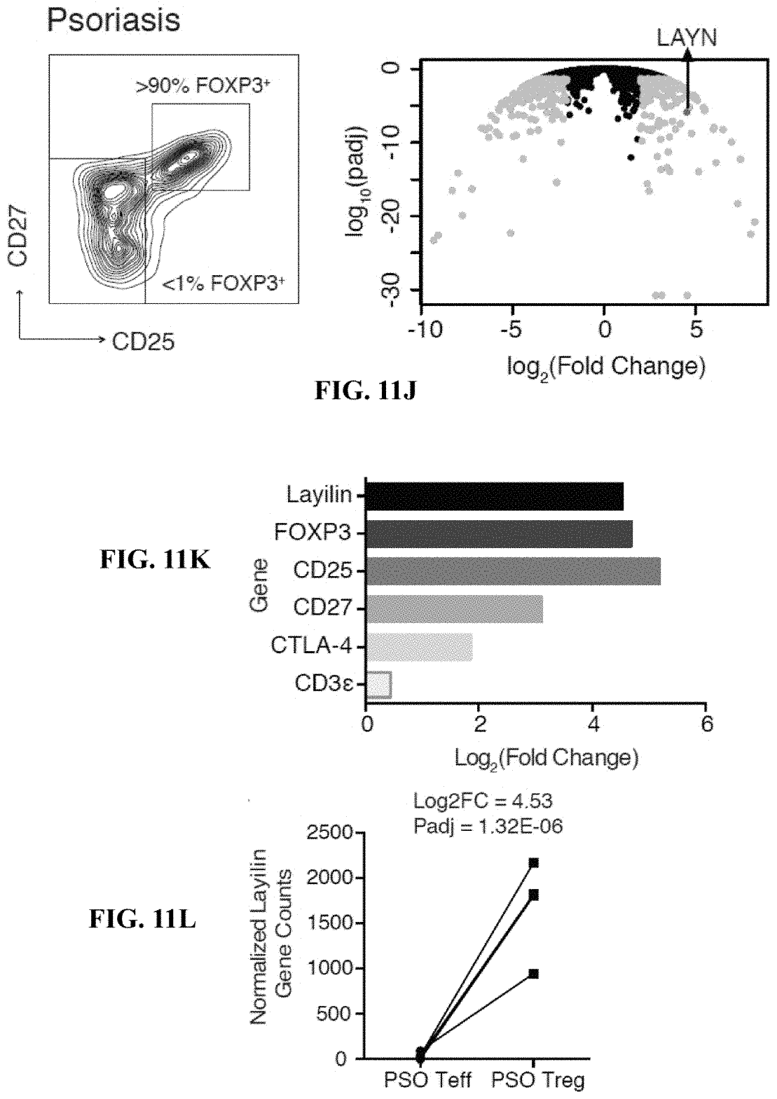

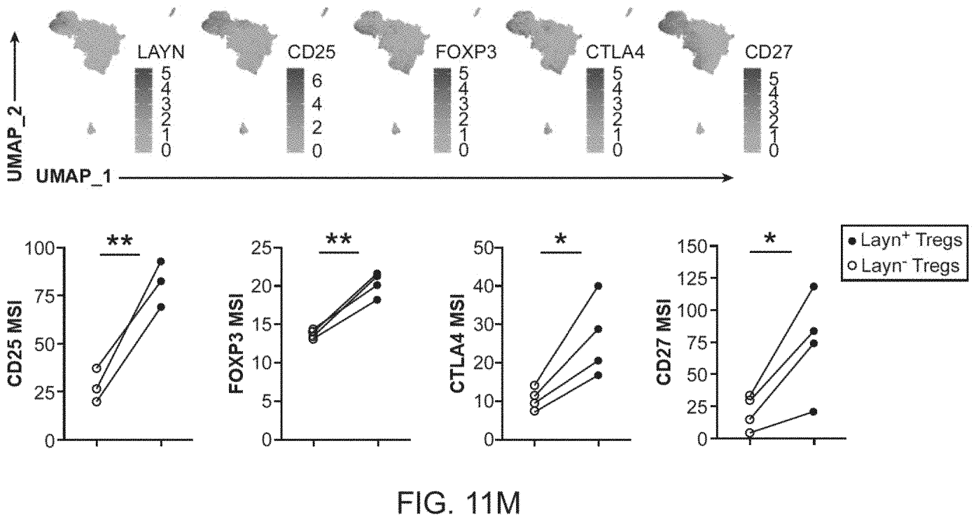

To elucidate molecular pathways that are unique to Tregs in human skin, whole transcriptome RNA sequencing (RNAseq) was performed on Tregs and CD4⁺ effector T (Teff) cells sort-purified from normal human skin (FIG. 11A). Using this unbiased discovery approach, LAYN was identified to be preferentially expressed by Tregs as compared to Teff cells in skin (FIG. 11A-C). The fold change in gene expression was comparable to that of Foxp3, the master regulator of Treg development and function (Hori et al., 2003). Differential expression of the ‘core Treg signature’ (Hill et al., 2007) between the two cell subsets, including CD25, CTLA-4 and CD27 to evaluate effective purification of Tregs (FIG. 11B and data not shown). To determine if layilin expression was unique to Tregs in human skin, Tregs, CD4⁺ Teff cells, CD8⁺ T cells, dendritic cells and keratinocytes were sort-purified from the skin of a separate cohort of normal healthy donors and performed whole transcriptome RNAseq analysis. Tregs preferentially express high levels of layilin when compared to all other cell populations evaluated (FIG. 11D). To validate our RNAseq findings, expression of layilin protein by flow cytometry on CD4⁺ T cells was measured in skin of normal healthy individuals and compared expression to these cells in peripheral blood (FIG. 11E). Consistent with our RNAseq results, layilin was highly expressed on skin Tregs compared to skin Teff cells. Although a small fraction of Tregs in peripheral blood expressed layilin (˜1-3%), approximately 40% of Tregs in skin expressed high levels of this protein (FIG. 11E). Interestingly, not all Tregs in skin expressed layilin at the protein level. This was not a result of enzymatic digestion of the epitope during skin cell preparation (data not shown), suggesting that only a subset of skin Tregs express layilin in normal human skin in the steady-state. To better define the layilin-expressing Treg subset, the expression of Treg activation/functional markers such FOXP3, CD25, CTLA4, ICOS and CD27 was quantified on layilin⁺ and layilin⁻ Tregs in healthy human skin. Layilin⁺ Tregs expressed significantly higher levels of all these Treg ‘effector’ molecules (FIG. 11F).

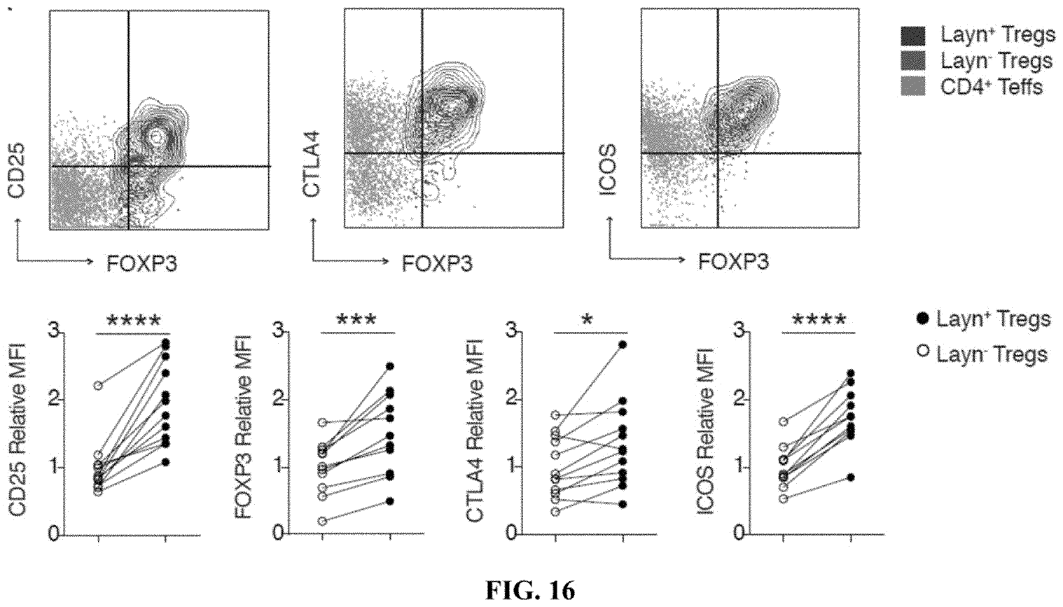

To determine if layilin expression was maintained on Tregs in diseased human skin, tumors from patients with metastatic melanoma and skin of patients with psoriasis were analyzed. Whole transcriptome RNAseq was performed on sort-purified Tregs and Teff cells in a similar fashion to that described for normal skin. Tregs infiltrating metastatic melanoma tumors and psoriasis skin express significantly higher levels of layilin as compared to CD4⁺ Teff cells (FIG. 11G-L). Mass cytometric (CyTOF) analysis of immune cell infiltrates in psoriatic skin revealed that layilin expression correlated with the most ‘activated’ Tregs (FIG. 11M). Similar findings were observed on Tregs infiltrating human melanoma, as quantified by standard flow cytometry (FIG. 16A). Taken together, these results suggest that layilin is preferentially expressed on a subset of highly activated Tregs in healthy and diseased human skin, with minimal expression on Tregs in peripheral blood and other immune and non-immune cell types in human skin.

### Example 11 Layilin Attenuates Treg Activation and Suppressive Capacity In Vitro

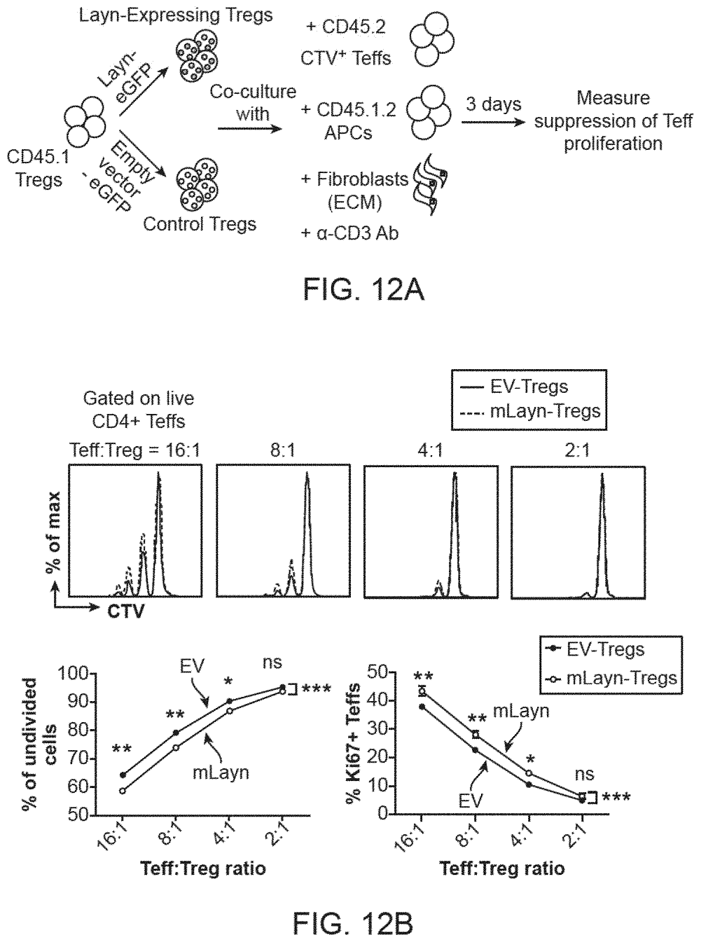

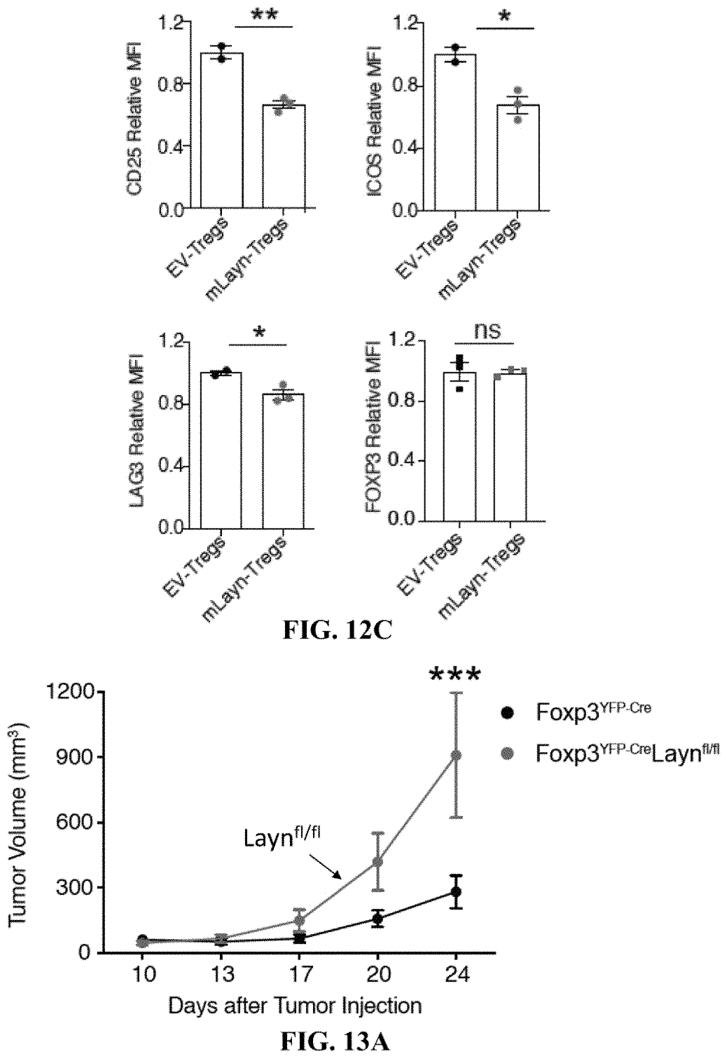

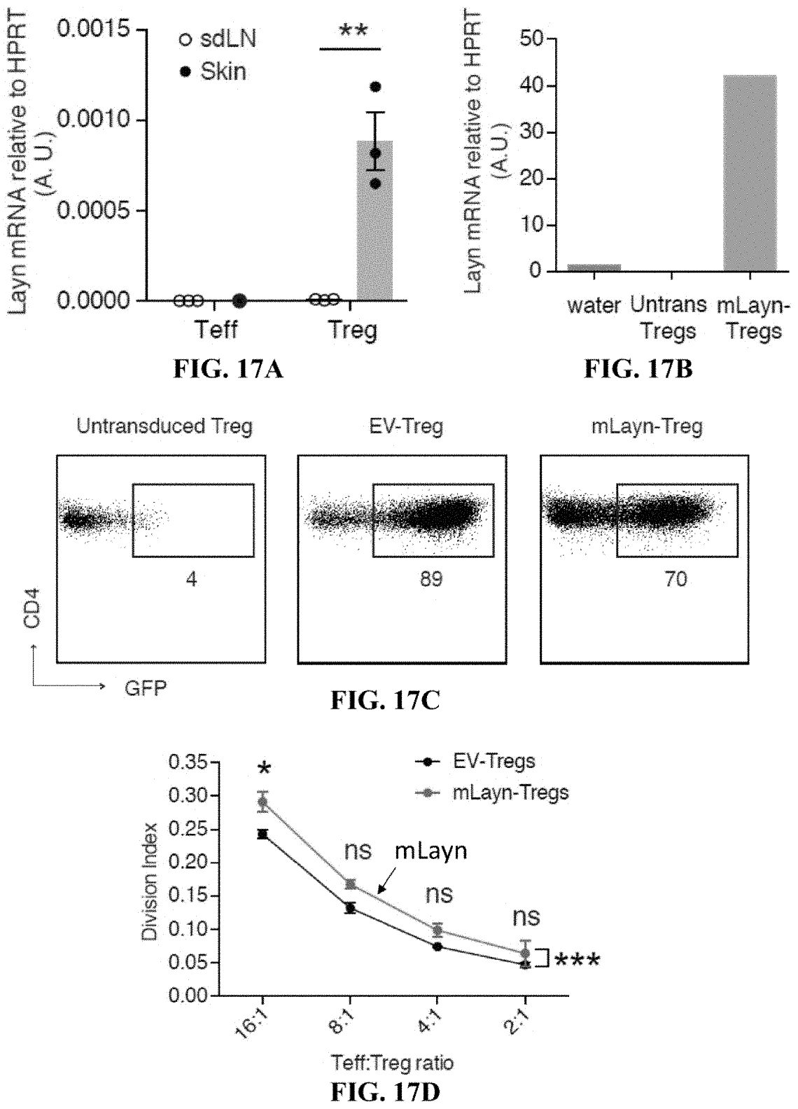

To determine if layilin influences Treg suppressive capacity, layilin protein was overexpressed on murine Tregs. Consistent with the finding that layilin is minimally expressed on Tregs in human peripheral blood (FIG. 11E), Tregs isolated and expanded from murine secondary lymphoid organs (i.e., spleen and lymph nodes) express minimal amounts of layilin (FIG. 17A & S2C), thus providing an ideal cell source to determine how induced layilin expression influences Treg function. In these experiments, a retroviral transduction approach was employed to express mouse layilin on Tregs (mLayn-Tregs) isolated from skin draining lymph nodes (sdLN) and spleen. Control Tregs were transduced with empty vector-eGFP (EV-Tregs). The efficiency of transduction was routinely ˜70-90%, as measured by GFP expression and mLayn-transduced cells expressed significantly higher levels of layilin mRNA when compared to untransduced Tregs (FIGS. 17B & C). Congenically disparate CellTrace Violet (CTV)-labeled CD4+ Teffs were stimulated with anti-CD3 and irradiated APCs in the presence of mLayn-Tregs or control EV-Tregs. These assays were performed on plates pre-coated with syngeneic dermal fibroblasts, to provide extracellular matrix as a physiologic ligand for layilin (Bono et al., 2001) (FIG. 12A). Tregs over-expressing layilin had reduced suppressive capacity with increasing Treg to Teff ratios (FIG. 12B). Accordingly, proliferation of Teffs, as measured by Ki67 staining and CTV-based division index, was found to be significantly higher in Teffs cocultured with mLayn-Tregs (FIG. 12B and FIG. 17D). To determine if layilin expression attenuates Treg activation, mLayn-Tregs and EV-Tregs were cocultured with APCs and anti-CD3, in the absence of Teff cells. Consistent with the suppression data, there was a significant reduction in expression of CD25, ICOS and LAG3 on layilin expressing Tregs (FIG. 12C). Interestingly, layilin expression did not affect Foxp3 levels in these assays (FIG. 12C). Taken together, these results suggest that layilin expression on Tregs attenuates expression of select activation markers and reduces their capacity to suppress Teff cell proliferation in vitro.

### Example 12 Layilin Attenuates Treg Suppressive Capacity In Vivo

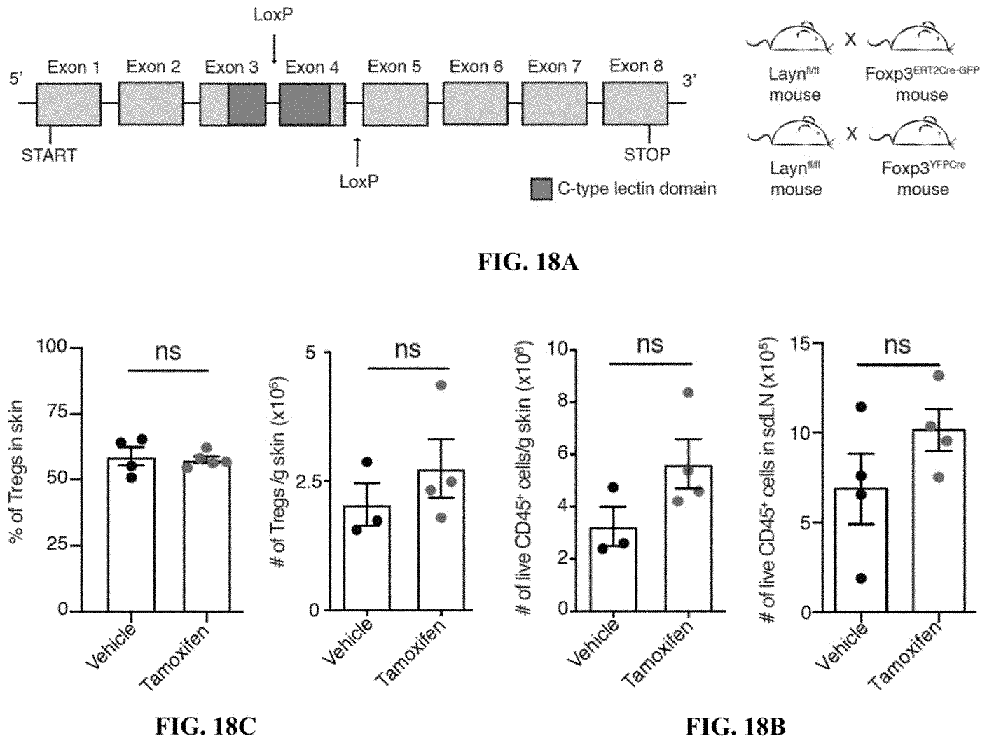

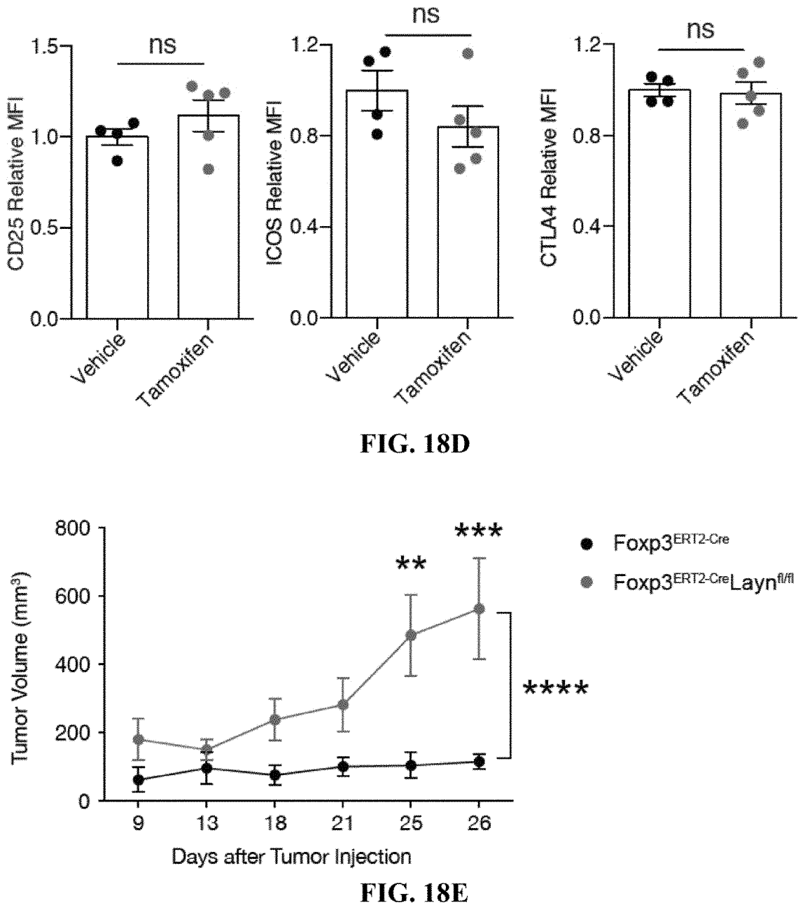

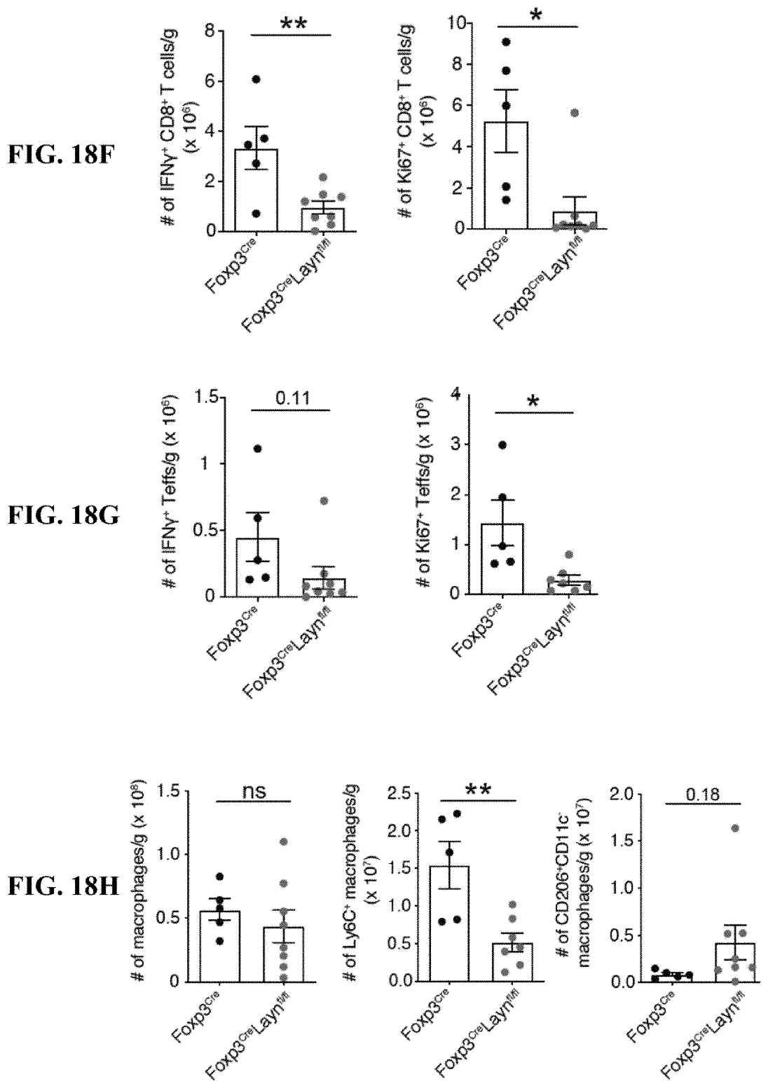

To determine if layilin influences Treg suppressive capacity in vivo, expression in mice mirrored that of humans was confirmed, with expression on skin Tregs and minimal expression on Tregs in secondary lymphoid organs and skin Teff cells (FIG. 17A). Next, a mouse strain in which Layn could be conditionally deleted in specific cell types was generated (i.e., Layn^{flox/flox} mice). Flox sequences were inserted to flank exon 4 of the layilin gene using CRISPR/Cas9 technology (Cong et al., 2013a). This results in complete deletion of exon 4, corresponding to the C-type lectin domain of LAYN, when crossed to mice expressing Cre-recombinase in specific cell lineages (Borowsky and Hynes, 1998b) (FIG. 18A). To elucidate the function of layilin on Tregs, Layn^{flow/flow} mice were crossed to Foxp3^{YFP-Cre} mice (Rubtsov et al., 2008) (Foxp3^{Cre}Layn^{fl/fl}) or Foxp3^{ERT2-GFP-Cre} mice (Rubtsov et al., 2010) (Foxp3^{ERT2-Cre} Layn^{fl/fl}) in which layilin is deleted in Tregs throughout development or can be induced to be deleted in adult animals (upon treatment with tamoxifen), respectively (FIG. 18A). Both Foxp3^{Cre}Layn^{fl/fl} and Foxp3^{ERT2-Cre}Layn^{fl/fl} mice developed normally and did not have any gross defects in total leukocyte numbers in lymphoid organs and peripheral non-lymphoid organs (FIG. 18B-D and data not shown). Treg numbers and phenotype in skin and other peripheral organs in Foxp3^{ERT2-Cre}Layn^{fl/fl} mice after treatment with tamoxifen were normal when compared to untreated gender- and age-matched control mice (FIG. 18B-D and data not shown).

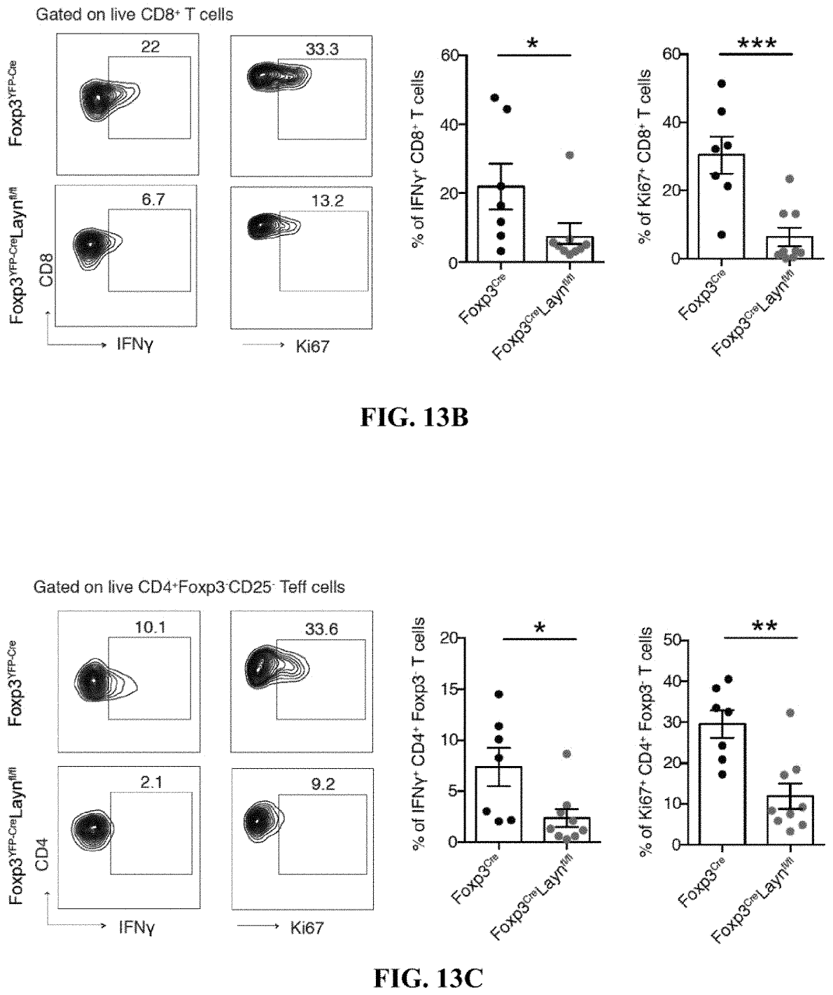

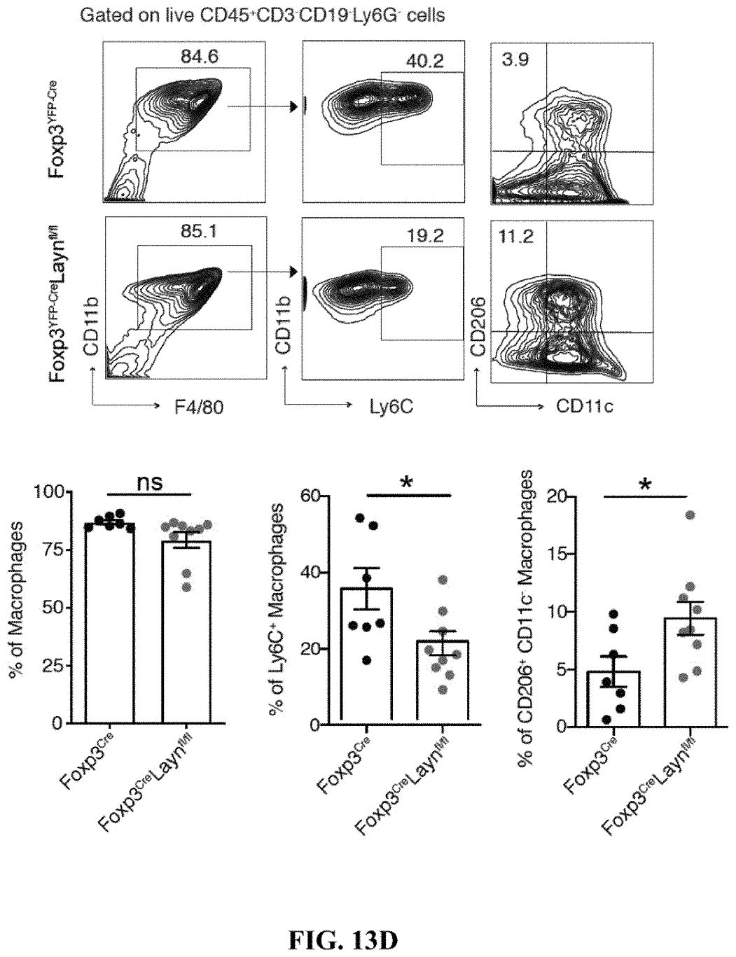

Because layilin is expressed on Tregs infiltrating human tumors (FIG. 11G-I and (De et al., 2016; Guo et al., 2018; Zheng et al., 2017)) and these cells have been shown to influence tumor growth and metastasis (Delgoffe et al., 2013; Nishikawa and Sakaguchi, 2010), the role for Treg expression of layilin influencing tumor growth was explored in the MC38 colon adenocarcinoma model. This model was chosen because it is relatively immunoresponsive where Tregs play a significant role (Delgoffe et al., 2013; Nishikawa and Sakaguchi, 2010). When compared to Foxp3^{Cre} control mice, Foxp3^{Cre}Layn^{fl/fl} mice had significantly increased tumor volumes and growth kinetics (FIG. 13A). Similar results were observed in Foxp3^{ERT2-Cre}Layn^{fl/fl} mice upon treatment with tamoxifen when compared to untreated age- and gender-matched littermate controls (FIG. 18E). Quantification of tumor immune cell infiltrates revealed a significant reduction in IFNγ-producing CD8⁺ T cells and reduced proliferative (Ki67⁺) CD8⁺ T cells in Foxp3^{Cre}Layn^{fl/fl} mice compared to controls (FIG. 13B and FIG. 18F). Similar results were observed in the CD4⁺ Teff compartment (FIG. 13C and FIG. 18G). In addition, Ly6C^{high} pro-inflammatory tumor-infiltrating macrophages were significantly reduced in Foxp3^{Cre}Layn^{fl/fl} mice with a concomitant increase in CD206^{high} anti-inflammatory macrophages (FIG. 13D and FIG. 18H). These results are consistent with and expand upon our in vitro data, suggesting that layilin expression on Tregs attenuates their capacity to regulate inflammation in tissues.

### Example 13 Layilin Expression on Tregs Enhances their Accumulation in Tissues

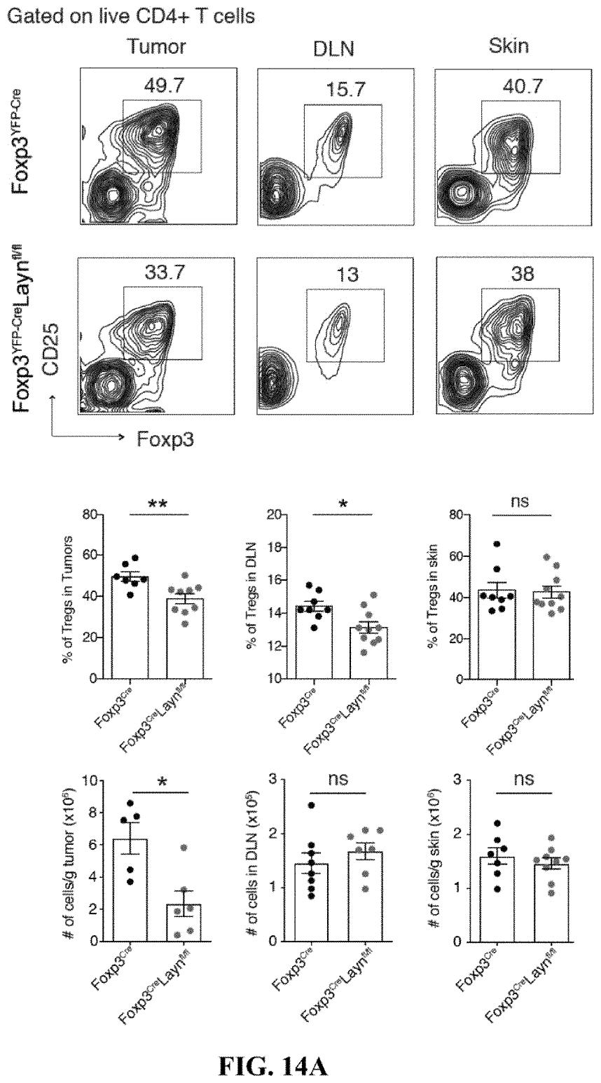

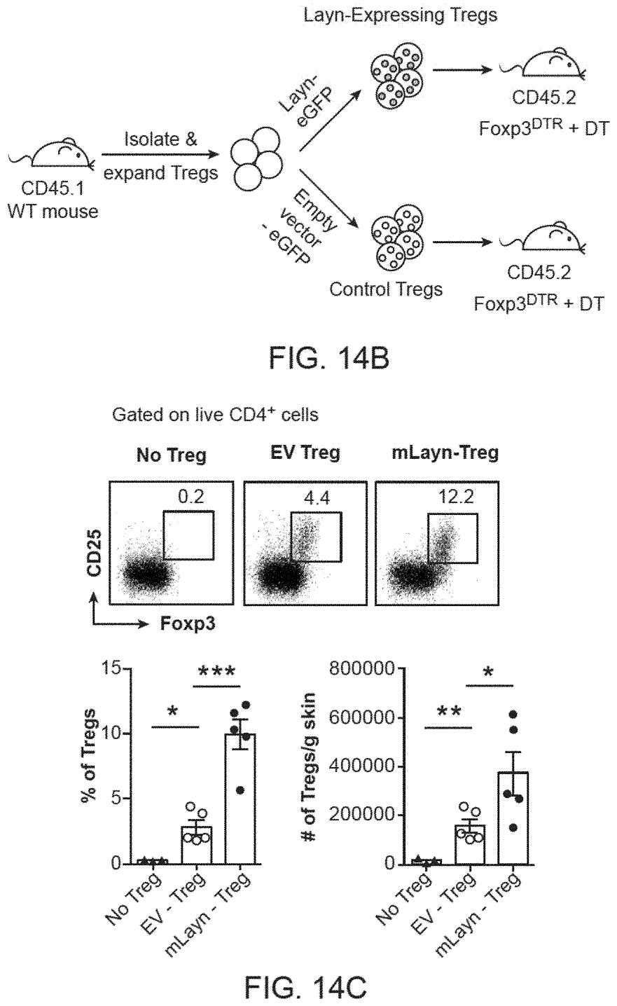

Layilin has been shown to mediate epithelial cell adhesion to the extracellular matrix in vitro (Borowsky and Hynes, 1998a; Chen et al., 2008). However, as far as currently known, this has yet to be demonstrated in vivo. In addition, mice with layilin deleted specifically in Tregs have no gross abnormalities (FIG. 18 and data not shown), suggesting that this molecule may not play a significant role in Treg adhesion in the steady-state. To begin to test whether layilin influences Treg adhesion in vivo, Treg accumulation in tumors in the MC38 model was quantified. Consistent with a role in cellular adhesion, layilin-deficient Tregs (in Foxp3^{Cre}Layn^{fl/fl} mice) were reduced in percentage and absolute numbers in tumors when compared to control mice (FIG. 14A). This was primarily observed in tumors, as there were no differences in absolute numbers of Tregs in tumor draining lymph nodes (DLNs) and adjacent uninvolved skin between Foxp3^{Cre}Layn^{fl/fl} mice and Foxp3^{Cre} controls (FIG. 14A). There was a slight decrease in the percentage of Tregs in tumor DLNs in Foxp3^{Cre}Layn^{fl/fl} mice (FIG. 14A). Taken together, these results suggest that layilin expression on Tregs facilitates their accumulation in tumors. However, layilin expressing Tregs are less suppressive, resulting in a cumulative reduction in immune regulation with a net increase in activated immune cells in the tumor microenvironment and reduced tumor growth.

Layilin mediated accumulation of Tregs in tumors may be secondary to enhanced Treg migration, proliferation, survival and/or adhesion. In an attempt to functionally discern between these in vivo, a well-established Treg adoptive transfer model into Foxp3-DTR hosts was utilized (Delacher et al., 2020; van et al., 2016; Wyss et al., 2016). In this model, endogenous Tregs are depleted through administration of diphtheria toxin and syngeneic Tregs adoptively transferred to replenish the Treg compartment in secondary lymphoid organs and peripheral tissues. mLayn- or EV-transduced Tregs (isolated and expanded from secondary lymphoid organs as described above) were adoptively transferred into Foxp3^{DTR} mice (Kim et al., 2007) and Tregs were depleted for 10 days. Skin was then harvested for flow cytometric quantification of relative Treg abundance (FIG. 14B). Metrics of Treg proliferation and survival were also assessed. A pronounced and significant increase in the accumulation of mLayn-Tregs was observed in skin compared to control EV-Tregs (FIG. 14C). There was a preferential accumulation of transduced (i.e., GFP⁺) cells in the total CD45.1⁺ transferred population in the mLayn-transduced group compared to the EV-transduced control group (FIG. 14D), suggesting that layilin expression (and not the transduction process itself) correlates with increased tissue Treg accumulation. Interestingly, differences in the proliferative index (as measured by percentage of Tregs expressing Ki67) between mLayn- and EV-transduced Tregs either early or late post-transfer was not observed (FIG. 14E and data not shown). In addition, the percentage of dead cells within the CD45.1⁺ gate was equal between the two groups both early and late post-adoptive transfer (data not shown). These results suggest that migration to and/or retention in skin is the primary mechanism by which layilin-expressing Tregs preferentially accumulate.

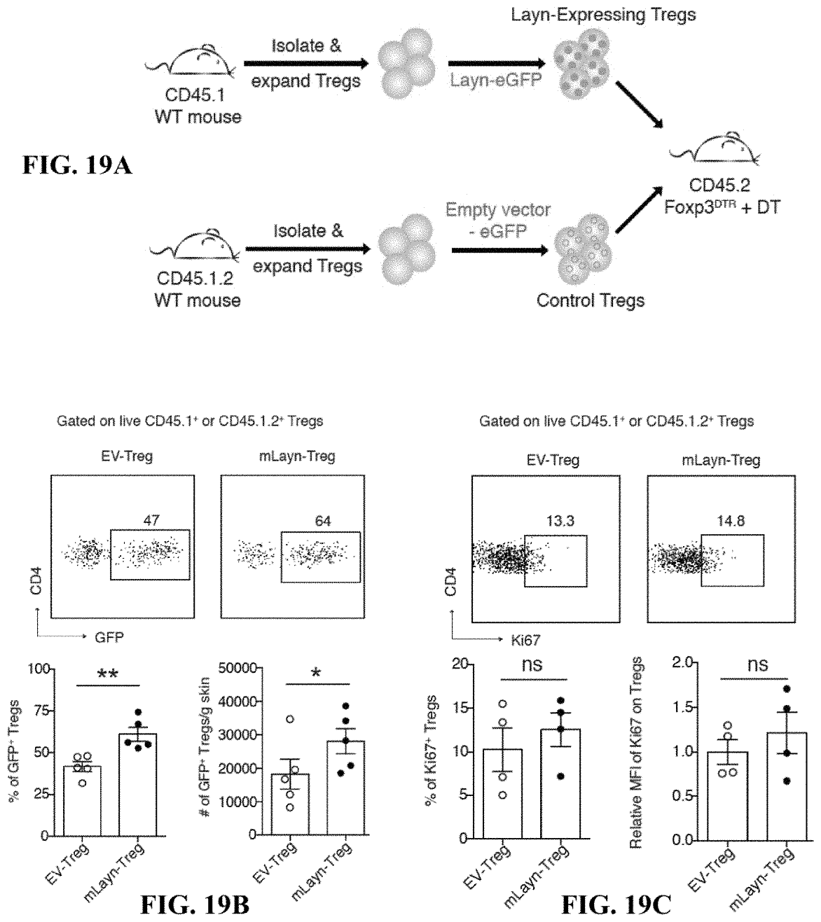

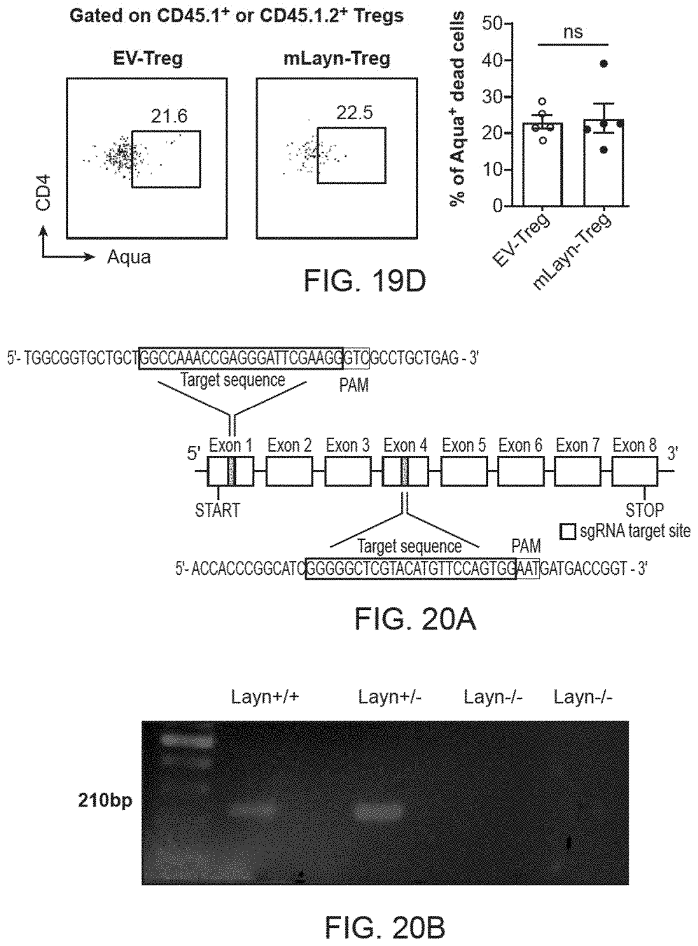

Layilin expressing Tregs are less suppressive (FIGS. 12 and 13). Thus, enhanced Treg accumulation in the experiments described above could be secondary to a more inflammatory environment created by layilin-expressing cells. To test whether layilin-mediated Treg accumulation was cell-intrinsic or dependent on the tissue microenvironment, competitive adoptive transfer experiments were performed. Congenically labeled mLayn- and EV-transduced Tregs were mixed in a 1:1 ratio and co-adoptively transferred into the same Foxp3^{DTR} host mice depleted of endogenous Tregs (FIG. 19A). After 10 days of Treg depletion, skin was harvested and Treg accumulation quantified by flow cytometry. Consistent with experiments where Tregs were transferred into separate hosts, significantly enhanced accumulation of layilin-expressing Tregs relative to EV controls in skin of co-adoptively transferred animals was observed (FIG. 19B). Additionally, there was no significant difference in Ki67 expression between mLayn- and EV-transduced Tregs either early or late after adoptive transfer (FIG. 19C and data not shown). There was also no significant difference in the percentage of dead Tregs between the 2 cell populations (FIG. 19D). Taken together, these results suggest that layilin promotes the in vivo accumulation of Tregs in tissues in a cell-intrinsic fashion, and that this is most likely not secondary to enhanced proliferation or survival.

### Example 14—Layilin Functions to ‘Anchor’ Tregs in Tissues

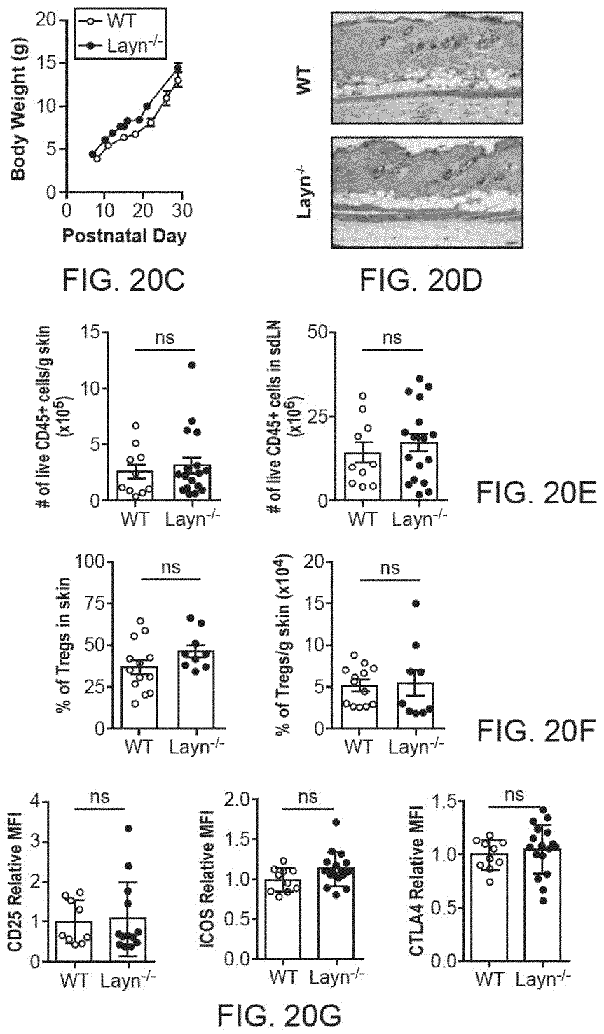

To further discern the mechanism by which layilin influences Treg accumulation in skin, intravital tissue imaging of these cells was performed. Because the YFP and GFP intensities in Foxp3^{Cre} and Foxp3^{ERT2-Cre} mice are too weak to be reliably detected by 2-photon microscopy, mice with a germline deletion of layilin were generated and crossed to Foxp3-GFP reporter mice (Lin et al., 2007). Layilin-deficient mice (Layn^{--/--}) were created using CRISPR-Cas9 gene editing of C57BL/6 embryos (Cong et al., 2013b). The single guide RNAs were designed against exon 1 and 4 and gene deletion in murine founder lines (backcrossed >2 generations to wildtype C57BL/6 mice) confirmed by layilin-specific PCR (FIG. 20A-B). Layn^{--/--} mice had normal-sized litters with no gross abnormalities in growth or development (FIG. 20C). There were no obvious signs of spontaneous autoimmune disease and skin morphology appeared similar to WT mice (FIG. 20D). The percentage and absolute numbers of total CD45⁺ leukocytes as well as Tregs in skin and secondary lymphoid organs of Layn^{--/--} mice revealed no abnormalities when compared to gender- and age-matched wildtype control animals (FIG. 20E-F). Additionally, there were no significant differences in expression of Treg activation markers, including CD25, ICOS, and CTLA-4, between Layn^{--/--} and WT mice skin (FIG. 20G). Similar results were observed in LN and spleen (data not shown).

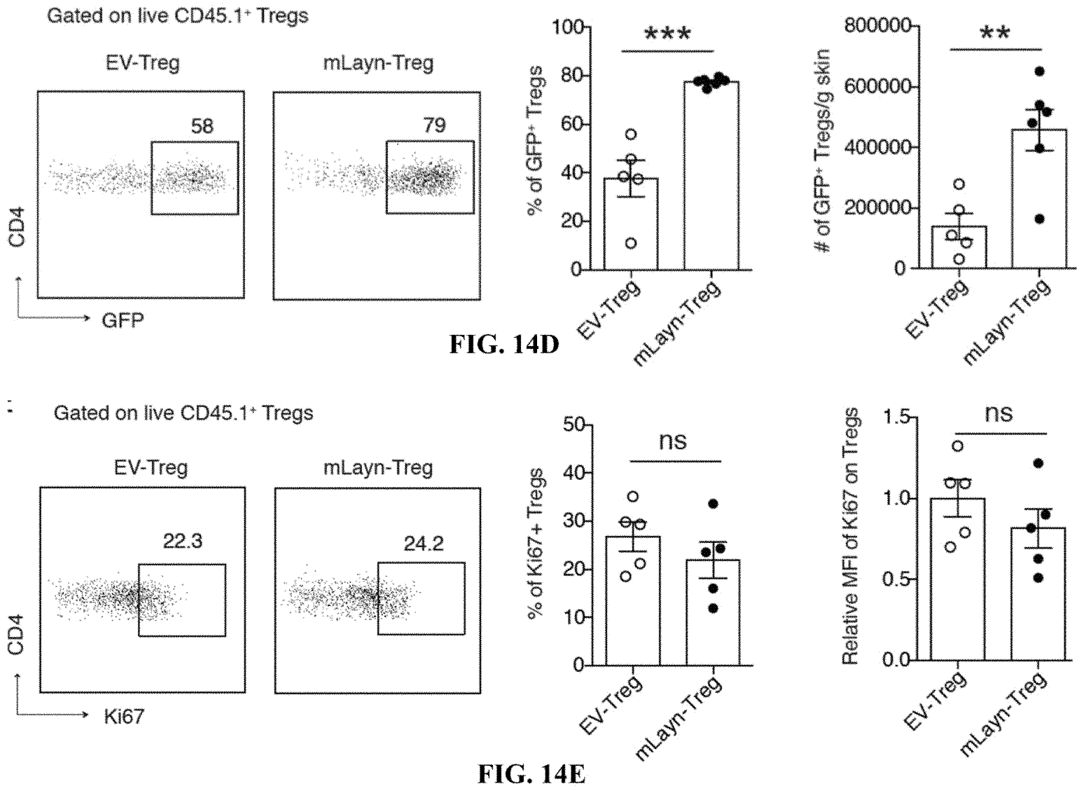

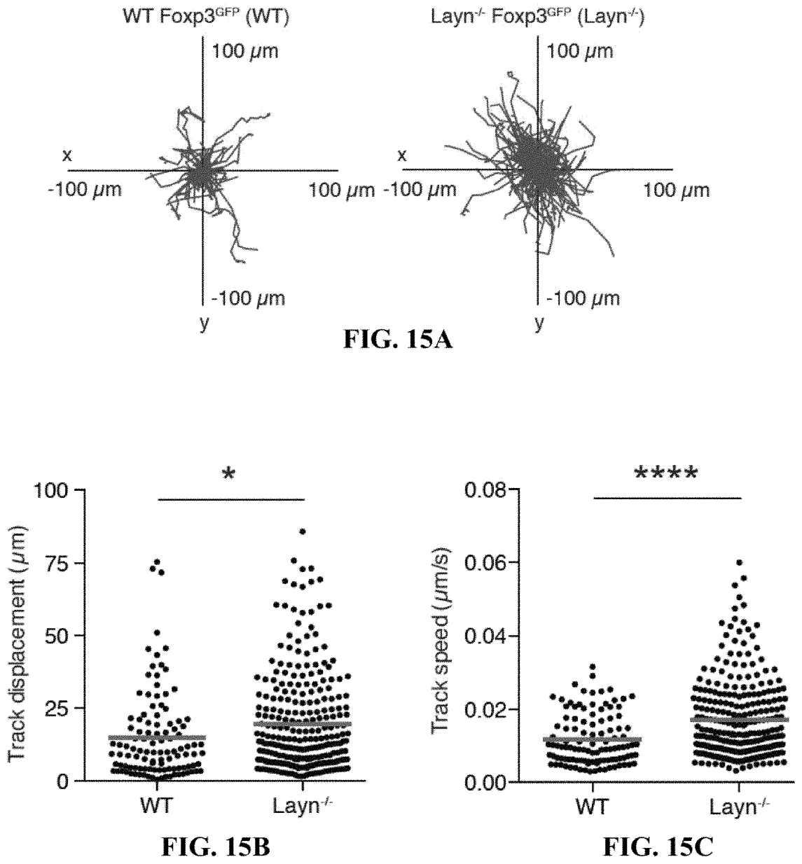

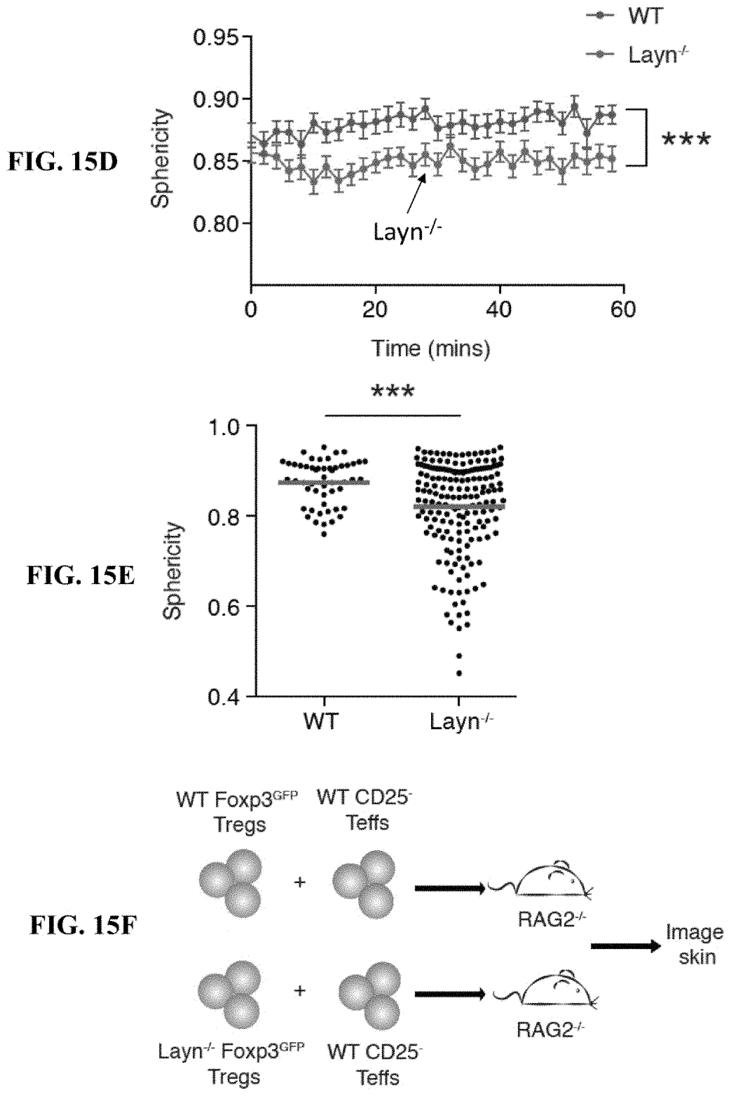

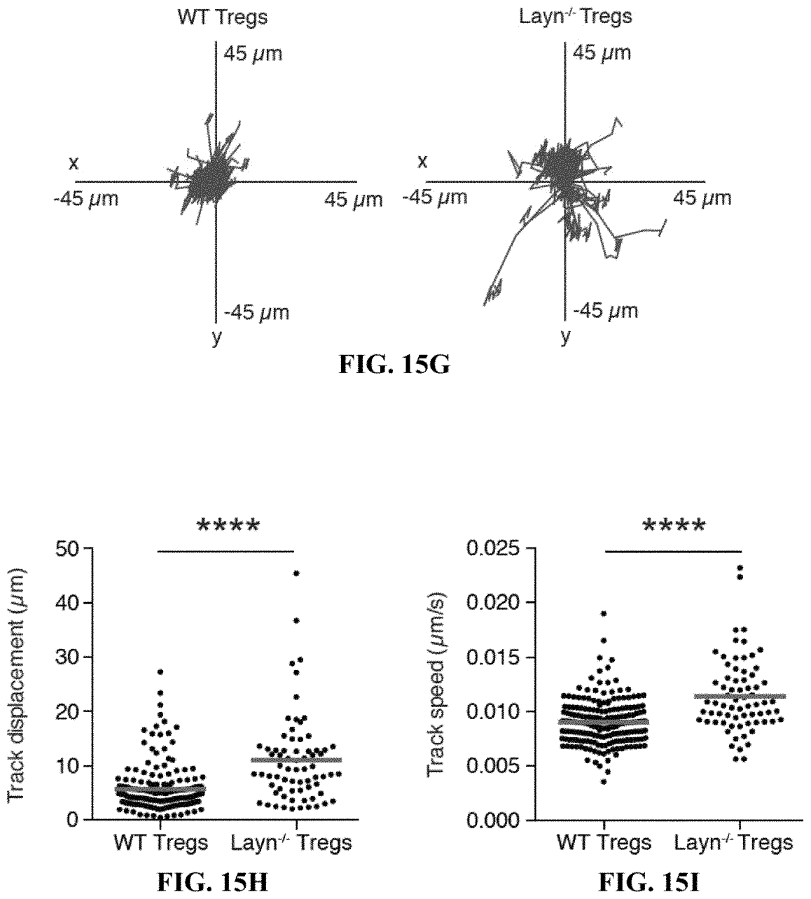

To test whether layilin expression influences the dynamic motility of Tregs in skin, intravital 2-photon microscopy was performed on Layn^{--/--} Foxp3^{GFP} mice. A unique, recently established vacuum suction approach (Ali et al., 2017) was utilized for imaging intact dorsal skin. Mice were imaged at 8-10 weeks of age, a time point when there are maximum number of Tregs in skin of adult animals (Ali et al., 2017). When compared to control WT Foxp3GFP mice, Tregs in dorsal skin of Layn^{--/--} mice travelled longer distances at increased speeds, as measured by track displacement length and track speed mean (FIG. 15A-C). Reduced sphericity is a marker of increased cell motility (Lecuit and Lenne, 2007). Layn^{--/--} Tregs exhibited a more amoeboid-like morphology with increased protrusive activity (data not shown). These differences in cell shape were quantified using Imaris software by rendering 3D surfaces on Tregs and applying a measure of relative sphericity (Thornton et al., 2012). Layn^{--/--} Tregs had significantly reduced sphericity as compared to WT Tregs at all the time points measured with a proportionate reduction in mean sphericity (FIG. 15D-E). Taken together, these results indicate that Tregs in Layn^{--/--} mice are less adherent and have increased motility in skin.

Because the experiments described above were performed in germline layn^{--/--} mice, it is possible that layilin deficiency on a cell subset other than Tregs resulted in the observed differences in Treg motility. To determine if layilin expression on Tregs influences the motility of these cells in a cell-intrinsic fashion, adoptive transfer experiments was performed with Layn^{--/--} Tregs. Immunodeficient RAG2^{--/--} mice were adoptively transferred with Tregs from either Layn^{--/--} Foxp3GFP mice or WT Foxp3^{GFP} controls, along with WT CD4⁺ Teff cells as a source of IL-2 needed for Treg survival in this model (Duarte et al., 2009) (FIG. 15F). Four to six weeks later, the skin of recipient mice was imaged using the intravital 2-photon approach described above (data not shown). Consistent with experiments performed in Layn^{--/--}/Foxp3GFP mice, the results demonstrated that Layn^{--/--} Tregs had significantly increased track displacement length and track speed mean as compared to WT Tregs (FIG. 15G-I). These results validate experiments performed in Layn^{--/--} mice and suggest that layilin expression on Tregs promotes their anchoring and adhesion in skin, which may help in promoting their accumulation in tissues.

### Methods and Materials for Examples 10-14

Experimental Animals

C57BL/6J wild-type (WT), Foxp3^{DTR} mice, Foxp3^{GFP}, CD45.1, Foxp3^{YFPCre}, Foxp3^{ERT2-GFPCre} and Rag2^{--/--} mice were purchased from The Jackson Laboratory (Bar Harbor, Me.) and were bred and maintained in the University of California San Francisco (UCSF) specific pathogen-free facility. Mice with a germ-line deletion of layilin (Layn^{--/--}) were created using a CRISPR-Cas9 approach (Cong et al., 2013b). Guide RNAs were designed to target exons 1 and 4 and delivered with Cas9 into C57BL/6 embryos (FIG. 17A). Three founder lines were generated: 2 with deletions from exon 1 to 4 and one with a SNP in exon 4, resulting in a premature stop codon. Founder pups generated were back-crossed to wildtype C57BL/6 mice (over 2 generations) to establish layilin-deficient (Layn^{--/--}) mouse lines. Layn^{fl/fl} mice were created by inserting LoxP sites flanking exon 4 of layilin gene using CRISPR-Cas9. Layilin was deleted specifically on Tregs by crossing Layn^{fl/fl} mice to Foxp^{YFPCre} mice or Foxp3^{ERT2-GFPCre} mice, upon treatment with tamoxifen. All mouse experiments were performed on 7-12 week old animals. All mice were housed under a 12 hour light/dark cycle. All animal experiments were performed in accordance with guidelines established by Laboratory Animal Resource Center at UCSF and all experimental plans and protocols were approved by IACUC beforehand.

Human Specimens

Normal healthy human skin was obtained from patients at UCSF undergoing elective surgery, in which as a routine procedure, healthy skin was discarded. Blood samples were obtained from healthy adult volunteers (study number 12-09489). Biopsies of accessible melanoma tumors were obtained with a 16- or 18-gauge needle, or a 4-mm punch biopsy tool (study number 138510). Studies using human samples were approved by the UCSF Committee on Human Research and by the IRB of UCSF. Informed written consent was obtained from all patients.

Human Skin Digestion

Skin samples were stored in a sterile container on gauze and PBS at 4° C. until the time of digestion. Skin was processed and digested as previously described (Sanchez et al., 2014). Briefly, hair and subcutaneous fat were removed, and skin was cut into small pieces and mixed with digestion buffer containing 0.8 mg/ml Collagenase Type 4 (4188; Worthington), 0.02 mg/ml DNAse (DN25-1G; Sigma-Aldrich), 10% FBS, 1% HEPES, and 1% penicillin/streptavidin in RPMI medium and digested overnight in an incubator. They were then washed (2% FBS, 1% penicillin/streptavidin in RPMI medium), double filtered through a 100-μm filter, and cells were pelleted and counted. Human PBMCs were prepared by Ficoll-Paque gradient centrifugation. Single cell suspensions were then stained with antibodies for flow cytometric analysis or FACS sorting.

RNA-Sequencing Analysis of Tregs and Teff Cells

Treg cells were isolated by gating on live CD45⁺ CD3⁺ CD4⁺ CD8⁻ CD25^{hi}CD27^{hi} cells, which contained greater than 90% Foxp3-expressing Tregs. Teff cells were isolated by gating on live CD45⁺ CD3⁺ CD4⁺ CD8CD25^{low}CD27^{low} cells, which contained less than 1% Foxp3-expressing Tregs. Sort-purified cell populations were flash frozen in liquid nitrogen and were shipped overnight on dry ice to Expression Analysis, Quintiles (Morrisville, N.C.). RNA samples were converted into cDNA libraries using the Illumina TruSeq Stranded mRNA sample preparation kit. (Illumina). RNA was isolated using Qiagen RNeasy Spin Column and was quantified via Nanodrop ND-8000 spectrophotometer. The quality of RNA was checked using Agilent Bioanalyzer Pico Chip. 220 μg of input RNA was used to create cDNA using the SMARTer Ultra Low input kit. Samples were sequenced using Illumina RNA-Seq to a 25M read depth. Reads were aligned to Ensembl hg19 GRCh37.75 reference genome using TopHat software (v. 2.0.12) (Trapnell et al., 2009) and SAM files were generated using SAMtools (Li et al., 2009). Read counts were obtained with htseq-count (0.6.1p1) with the union option (Anders et al., 2015). The R/Bioconducter package DESeq2 was used to determine differential expression (Love et al., 2014).

RNA-Sequencing Analysis of Tregs, Teff Cells, CD8⁺ T Cells, Dendritic Cells and Keratinocytes from Healthy Human Skin

Cells were sorted and analyzed as described previously (Ahn et al., 2017). Tregs and Teffs were sorted as described above. Expression of layilin was analyzed by ANOVA.

Mass Cytometry

Single cell suspensions were obtained from 4 mm punch biopsies of psoriatic lesions. Cells were first washed with 5 mM EDTA-PBS and centrifuged at 600 g for 5 minutes at 4° C. Cells were then resuspended with equal volumes of 5 mM EDTA-PBS and 50 uM cisplatin (Sigma, P4394) for 1 minute at room temperature (RT) before quenching with 5 mM EDTA-PBS with 0.5% BSA. After centrifugation, cells were fixed with 1.6% PFA in PBS with 0.5% BSA and 5 mM EDTA for 10 minutes at RT and then washed twice with PBS. Cells were then resuspended in PBS with 0.5% BSA and 10% DMSO and stored at −80° C. Prior to staining, cells were left to thaw at RT and washed in Cell Staining Media (CSM, PBS with 0.5% BSA and 0.02% NaN3) and then vortexed with FC Receptor Blocking Solution (BioLegend, 422302). LAYN (Sino Biological, 10208-MM02), PD-1 (BioLegend, EH12.2H7), and CD8a (BioLegend, RPA-T8) antibodies were metal-conjugated at the UCSF Parnassus Flow Cytometry Core using Maxpar Antibody Labeling Kits (Fluidigm). All other metal conjugated antibodies were obtained from Fluidigm. Cells were stained as previously described (Spitzer et al., 2015). Briefly, cells were stained in an extracellular antibody cocktail for 30 minutes at RT on a shaker and then washed with CSM. Cells were then permeabilized with the Foxp3/Transcription Factor Staining Buffer Set (eBioscience, 00-5523-00) for 30 minutes at RT on a shaker and then washed twice with Permeabilization Buffer (eBioscience, 00-8333-56) before staining in an intracellular antibody cocktail for 1 hour at RT on a shaker. Following intracellular staining, cells were washed once with Permeabilization Buffer and once with CSM, and then resuspended in PBS with 1.6% PFA and 100 nM Cell-ID Intercalator-Ir (Fluidigm, 201192B) and kept at 4° C. Before data acquisition, cells were washed sequentially in CSM, PBS, and MilliQ H₂O. Cells were then resuspended in MilliQ H₂O containing EQ Four Elements Calibration Beads (Fludigm, 201078) and analyzed with a CyTOF2 Mass Cytometer (Fluidigm). Mass cytometry files were normalized to the bead standards (Finck et al., 2013) in R (3.6.1) using the premessa package (0.2.4, github.com/ParkerICI/premesa). Analysis was performed on viable singlets as determined by the iridium, event length, and cisplatin channels. UMAP visualizations were generated with the CATALYST package (Nowicka et al., 2017) (1.10.1) using CD4+ cells (CD45+CD3+CD4+CD8−) exported manually from biaxial plots in FlowJo (10.6.1) and clusters were based on expression of CD25, FOXP3, CTLA4, CD27, and CD127.

Tumor Growth Experiments

MC38 colon adenocarcinoma model was performed as previously described (Collison et al., 2010). Briefly, 5×10⁵ MC38 tumor cells (Kerafast) resuspended in 200 ul of PBS were injected subcutaneously into the right flank of mice. Tumor diameters were measured every 2-3 days using electronic calipers and the tumor volume was calculated using the formula V=(L*W²)/2 (Faustino-Rocha et al., 2013). Tumor Infiltrating Lymphocytes (TILs) were isolated by harvesting tumors after 2-4 weeks, and mincing and digesting them similar to the skin.

Mouse Tissue Processing

Isolation of cells from axillary, brachial and inguinal lymph nodes (referred to as skin draining lymph nodes, sdLNs) and spleen for flow cytometry was performed by mashing tissue over sterile wire mesh. Mouse skin was digested and single cells suspensions prepared as previously described (Scharschmidt et al., 2015). Briefly, skin was minced and digested in buffer containing collagenase XI, DNase and hyaluronidase in complete RPMI in an incubator shaker at 225 rpm for 45 minutes at 37° C. An automated cell counter (NucleoCounter NC-200, Chemometec) was used to count cell numbers. 2-4×10⁶ cells were stained and flow cytometric analysis performed.

Flow Cytometry

Single-cell suspensions were counted, pelleted and incubated with anti-CD16/anti-CD32Fcblock (BD Bioscences; 2.4G2). Cells were washed and stained with Ghost Viability dye (Tonbo Biosciences) and antibodies against surface markers in PBS. For intracellular staining, cells were fixed and permeabilized using a FoxP3 staining kit (eBioscences) and then stained with antibodies against intracellular markers. Fluorophore-conjugated antibodies specific for human or mouse surface and intracellular antigens were purchased from BD Biosciences, eBiosciences or Biolegend. The following anti-mouse antibodies and clones were used: CD3 (145-2C11), CD4 (RM4-5), CD8 (53-6.7), CD45 (30-F11), FoxP3 (FJK-16s), TCRb (H57-597), CD25 (PC61.5), CD45.1 (A20), CD45.2 (104), CTLA4 (UC10-4B9), ICOS (C398.4A), Ki67 (B56), IFNγ (XMG1.2), TNFα (MP6-XT22), Ly6G (1A8), F4/80 (BM8), CD11b (M1/70), MHC class II (M5/114.15.2), Ly6C (HK1.4), CD206 (C068C2), CD11c (N418). The following anti-human antibodies and clones were used: layilin (LS Bio 4C11), CD3 (UCHT1), CD4 (SK3), CD8 (SK1), CD45 (HI30), FoxP3 (PCH101), CD25 (M-A251), CTLA4 (14D3), ICOS (ISA-3), CD27 (LG.7F9), CD11c (3.9), HLA-DR (L243). Samples were run on a Fortessa analyzer (BD Biosciences) in the UCSF Flow Cytometry Core and data was collected using FACS Diva software (BD Biosciences). Data were analyzed using FlowJo software (FlowJo, LLC). Dead cells and doublet cell populations were excluded, followed by pre-gating on CD45⁺ populations for immune cell analysis. Lymphoid cells were gated as TCRαβ⁺ CD3⁺αβ T cells, CD3⁺ CD8⁺ T cells (CD8), CD3⁺ CD4⁺ CD25⁻Foxp3⁻ T effector cells (Teff), and CD3⁺ CD4⁺ CD25⁺Foxp3⁺ regulatory T cells (Treg).

Ex Vivo Expansion and Retroviral Transduction of Mouse Tregs

Spleens and sdLN were harvested and lymphocytes isolated from congenically-marked CD45.1 C57BL/6 mice. Total CD4⁺ T cells were isolated using EasySep magnetic bead enrichment kit (StemCell Technologies). Tregs were sort-purified by gating on CD4⁺ CD25I¹ cells, which were

>95% Foxp3⁺, using Aria (BD Biosciences). In all experiments, purity of Tregs was >95%. Sorted Tregs were ex vivo expanded by methods previously described (Tang et al., 2004). Briefly, Tregs were cultured in complete DMEM with IL-2 (2000 U/ml, Tonbo Biosciences) and stimulated with mouse anti-CD3/CD28 beads at cells:beads ratio of 1:3 (Dynabeads, Thermo Fisher). On day 2, cells were retrovirally transduced with either control empty-eGFP-pMIG vector or Layilin-eGFP-pMIG vector at multiplicity of infection of 1 by spinoculation at 6000 g for 90 minutes at 25° C. Cells were then cultured and collected on day 5. On the day of collection, transduction efficiency (as measured by % of GFP⁺ cells) was checked by flow cytometry. Transduction efficiencies were routinely between 70% and 90% and were similar for empty vector and vector encoding Layilin. Also, an aliquot of cells were pelleted and frozen for later Layn mRNA analysis by qPCR.

In Vitro Mouse Treg Assays

To setup in vitro Treg suppression assay, sorted mouse Tregs, overexpressing either empty vector or Layilin-eGFP-pMIG vector, were cocultured with CellTrace Violet-labeled Teffs at varying proportions, along with mitomycin C-treated TCRb-depleted splenocytes (Antigen Presenting Cells) and soluble α-CD3ε (0.5 ug/ml) for 72 hours at 37° C. as previously described (Collison and Vignali, 2011). These experiments were carried out in triplicates/condition in a 96 well U-bottom plate precoated with mouse skin fibroblasts, as a potential source of ligand for layilin. Mouse skin fibroblasts were obtained by digesting the whole skin in presence of collagenase+ DNase and culturing the cells in fibroblast growth medium (Promocell) for 5-7 days to enrich for fibroblasts. Teffs were analyzed for CTV dilution by flow cytometry.

To setup in vitro Treg activation assay, Tregs overexpressing layilin or control vector were cocultured with APCs in presence of anti-CD3 Ab (0.5 ug/ml) without IL-2 for 72 hours at 37° C.

Adoptive Transfer of Layilin-Overexpressing Tregs into Foxp3DTR Mice

Cells were retrovirally transduced to overexpress layilin. 2.5 3.5×10⁵ cells re-suspended in PBS were adoptively transferred into Foxp3^{DTR} mice via retro-orbital injection. 3 days after adoptive transfer of cells, first Diphtheria toxin (DT) injection was given and then DT was injected every other day for a total of 5 doses. The optimal dose for each DT lot (Sigma-Aldrich) was previously determined by measuring the efficiency of skin Treg depletion by flow cytometry. Accordingly, Foxp3^{DTR} mice were injected with DT intraperitoneally at 30 ng/g body weight. Mice were sacrificed and skin and sdLN were harvested 13-14 days post-transfer.

Intravital Two-Photon Microscopy and Image Analysis

Instrumentation for two-photon imaging has been previously described (Bullen et al., 2009). Dorsal skin imaging using two-photon microscopy was done as previously described (Ali et al., 2017). Briefly, mice were anesthetized using isoflurane, hair on dorsal skin was shaved and depilated, and mice were then placed on a custom heated microscope stage. The depilated skin was gently immobilized using a custom suction window and an embedded 12 mm coverslip (Thornton et al., 2012). The microscope stage was then lifted to be right above a water-immersion objective lens (Olympus 25×, 1.05 numerical aperture). Fluorescence excitation was achieved by a Spectra-Physics MaiTai Ti-Saphire Laser tuned to 890 nm for excitation of GFP. Collagen was visualized using second harmonic signals. Z-stack images were acquired with a vertical resolution of 2 μm for a total of 80-100 μm depth. For collecting a time-series of images, three-dimensional stacks were acquired every 5 minutes using Micro-Magellan (Pinkard et al., 2016). Raw imaging data were processed using ImageJ Software. Images were analyzed and cells were tracked by rendering 3D surfaces and spots over the cells using Imaris Software (Bitplane). To determine in vivo changes in Treg cell shape, the sphericity of individual Tregs was calculated over the time-lapse period, as previously described (Thornton et al., 2012).

Quantitative PCR

For assessment of Layilin gene expression, Tregs and Teffs were sort-purified from skin and sdLNs of WT mice and RNA isolated using a column based kit (PureLink RNA Mini Kit, Thermo Fisher). RNA was then transcribed (iScript cDNA synthesis Kit, Bio-Rad) and pre-amplified (SSo Advanced PreAmp Supermix, Bio-Rad). Expression of Layilin was determined using a SYBR Green assay (SSo Advanced Universal SYBR Green kit; Biorad). Cycle number of duplicate or triplicate samples were normalized to the expression of the endogenous control β2m. Primer sequences or assay ids used are as follows: β2m (For: 5′ TTCTGGTGCTTGTCTCACTGA-3′ (SEQ ID NO: 11); Rev 5′ CAGTATGTTCGGCTTCCCATTC-3′ (SEQ ID NO: 12)), mouse Layilin (qMmuCID0022543, Biorad). Data are presented as negative fold change of Delta-Delta CT or as standardized arbitrary units (AU).

Statistical Analyses

Statistical analyses were performed with Prism software package version 6.0 (GraphPad). P values were calculated using two-tailed unpaired or paired Student's t-test, unless specified otherwise. Pilot experiments were used to determine sample size for animal experiments. No animals were excluded from analysis, unless due to technical errors. Mice were age- and gender-matched and randomly assigned into experimental groups. Appropriate statistical analyses were applied, assuming a normal sample distribution. All in vivo mouse experiments were conducted with at least 2-3 independent animal cohorts. RNA-Seq experiments were conducted using 4-5 biological samples (as indicated in figure legends). Data are mean±S.E.M. P values correlate with symbols as follows: ns=not significant, p>0.05, *p<0.05, **p<0.01, ***p<0.001, ****p<0.0001.

Other Sequences

Human Integrin-Beta 2 (UniProt Accession number
P05107), SEQ ID NO: 4
MLGLRPPLLALVGLLSLGCVLSQECTKFKVSSCRECIESGPGCTWCQKL
NFTGPGDPDSIRCDTRPQLLMRGCAADDIMDPTSLAETQEDHNGGQKQL
SPQKVTLYLRPGQAAAFNVTFRRAKGYPIDLYYLMDLSYSMLDDLRNVK
KLGGDLLRALNEITESGRIGFGSFVDKTVLPFVNTHPDKLRNPCPNKEK
ECQPPFAFRHVLKLTNNSNQFQTEVGKQLISGNLDAPEGGLDAMMQVAA
CPEEIGWRNVTRLLVFATDDGFHFAGDGKLGAILTPNDGRCHLEDNLYK
RSNEFDYPSVGQLAHKLAENNIQPIFAVTSRMVKTYEKLTEIIPKSAVG
ELSEDSSNVVQLIKNAYNKLSSRVFLDHNALPDTLKVTYDSFCSNGVTH
RNQPRGDCDGVQINVPITFQVKVTATECIQEQSFVIRALGFTDIVTVQV
LPQCECRCRDQSRDRSLCHGKGFLECGICRCDTGYIGKNCECQTQGRSS
QELEGSCRKDNNSIICSGLGDCVCGQCLCHTSDVPGKLIYGQYCECDTI
NCERYNGQVCGGPGRGLCFCGKCRCHPGFEGSACQCERTTEGCLNPRRV
ECSGRGRCRCNVCECHSGYQLPLCQECPGCPSPCGKYISCAECLKFEKG
PFGKNCSAACPGLQLSNNPVKGRTCKERDSEGCWVAYTLEQQDGMDRYL
IYVDESRECVAGPNIAAIVGGTVAGIVLIGILLLVIWKALIHLSDLREY
RRFEKEKLKSQWNNDNPLFKSATTTVMNPKFAES
Human Integrin-Alpha L (UniProt Accession number
P20701) SEQ ID NO: 5
MKDSCITVMAMALLSGFFFFAPASSYNLDVRGARSFSPPRAGRHFGYRV
LQVGNGVIVGAPGEGNSTGSLYQCQSGTGHCLPVTLRGSNYTSKYLGMT
LATDPTDGSILACDPGLSRTCDQNTYLSGLCYLFRQNLQGPMLQGRPGF
QECIKGNVDLVFLFDGSMSLQPDEFQKILDFMKDVMKKLSNTSYQFAAV
QFSTSYKTEFDFSDYVKRKDPDALLKHVKHMLLLTNTFGAINYVATEVF
REELGARPDATKVLIIITDGEATDSGNIDAAKDIIRYIIGIGKHFQTKE
SQETLHKFASKPASEFVKILDTFEKLKDLFTELQKKIYVIEGTSKQDLT
SFNMELSSSGISADLSRGHAVVGAVGAKDWAGGFLDLKADLQDDTFIGN
EPLTPEVRAGYLGYTVTWLPSRQKTSLLASGAPRYQHMGRVLLFQEPQG
GGHWSQVQTIHGTQIGSYFGGELCGVDVDQDGETELLLIGAPLFYGEQR
GGRVFIYQRRQLGFEEVSELQGDPGYPLGRFGEAITALTDINGDGLVDV
AVGAPLEEQGAVYIFNGRHGGLSPQPSQRIEGTQVLSGIQWFGRSIHGV
KDLEGDGLADVAVGAESQMIVLSSRPVVDMVTLMSFSPAEIPVHEVECS
YSTSNKMKEGVNITICFQIKSLIPQFQGRLVANLTYTLQLDGHRTRRRG
LFPGGRHELRRNIAVTTSMSCTDFSFHFPVCVQDLISPINVSLNFSLWE
EEGTPRDQRAQGKDIPPILRPSLHSETWEIPFEKNCGEDKKCEANLRVS
FSPARSRALRLTAFASLSVELSLSNLEEDAYWVQLDLHFPPGLSFRKVE
MLKPHSQIPVSCEELPEESRLLSRALSCNVSSPIFKAGHSVALQMMFNT
LVNSSWGDSVELHANVTCNNEDSDLLEDNSATTIIPILYPINILIQDQE
DSTLYVSFTPKGPKIHQVKHMYQVRIQPSIHDHNIPTLEAVVGVPQPPS
EGPITHQWSVQMEPPVPCHYEDLERLPDAAEPCLPGALFRCPVVFRQEI
LVQVIGTLELVGEIEASSMFSLCSSLSISFNSSKHFHLYGSNASLAQVV
MKVDVVYEKQMLYLYVLSGIGGLLLLLLIFIVLYKVGFFKRNLKEKMEA
GRGVPNGIPAEDSEQLASGQEAGDPGCLKPLHEKDSESGGGKD
Human Layilin (UniProt Accession number Q6UX15-1),
SEQ ID NO: 6
MRPGTALQAVLLAVLLVGLRAATGRLLSASDLDLRGGQPVCRGGTQRPC
YKVIYFHDTSRRLNFEEAKEACRRDGGQLVSIESEDEQKLIEKFIENLL
PSDGDFWIGLRRREEKQSNSTACQDLYAWTDGSISQFRNWYVDEPSCGS
EVCVVMYHQPSAPAGIGGPYMFQWNDDRCNMKNNFICKYSDEKPAVPSR
EAEGEETELTTPVLPEETQEEDAKKTFKESREAALNLAYILIPSIPLLL
LLVVTTVVCWVWICRKRKREQPDPSTKKQHTIWPSPHQGNSPDLEVYNV
IRKQSEADLAETRPDLKNISFRVCSGEATPDDMSCDYDNMAVNPSESGF
VTLVSVESGFVTNDIYEFSPDQMGRSKESGWVENEIYGY
Human Layilin (UniProt Accession number Q6UX15-2),
SEQ ID NO: 7
MRPGTALQAVLLAVLLVGLRAATGRLLSGQPVCRGGTQRPCYKVIYFHD
TSRRLNFEEAKEACRRDGGQLVSIESEDEQKLIEKFIENLLPSDGDFWI
GLRRREEKQSNSTACQDLYAWTDGSISQFRNWYVDEPSCGSEVCVVMYH
QPSAPAGIGGPYMFQWNDDRCNMKNNFICKYSDEKPAVPSREAEGEETE
LTTPVLPEETQEEDAKKTFKESREAALNLAYILIPSIPLLLLLVVTTVV
CWVWICRKRKREQPDPSTKKQHTIWPSPHQGNSPDLEVYNVIRKQSEAD
LAETRPDLKNISFRVCSGEATPDDMSCDYDNMAVNPSESGFVTLVSVES
GFVTNDIYEFSPDQMGRSKESGWVENEIYGY
Human Layilin (UniProt Accession number Q6UX15-3),
SEQ ID NO: 8
MVTSGLGSGGVRRNKAIAQPARTFMLGLMAAYHNLEKPAVPSREAEGEE
TELTTPVLPEETQEEDAKKTFKESREAALNLAYILIPSIPLLLLLVVTT
VVCWVWICRKRKREQPDPSTKKQHTIWPSPHQGNSPDLEVYNVIRKQSE
ADLAETRPDLKNISFRVCSGEATPDDMSCDYDNMAVNPSESGFVTLVSV
ESGFVTNDIYEFSPDQMGRSKESGWVENEIYGY

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A method for treating an autoimmune disorder in a subject in need thereof, comprising administering to the subject a therapeutically effective amount of a layilin-binding protein which inhibits the activity of layilin.

2. The method of claim 1, wherein the autoimmune disorder has a pathogenicity associated with the presence of CD8+ T cells in a diseased tissue.

3. The method of claim 1, wherein the layilin-binding protein is an anti-layilin antibody or a fragment thereof.

4. The method of claim 3, wherein the anti-layilin antibody is a full-length antibody, a Fab, a F(ab)₂, an Fv, a single chain Fv (scFv) antibody, a V_{H}, or a V_{H}H.

5. The method of claim 1, wherein the layilin-binding protein interferes with the binding of a beta integrin complex expressed on CD8+ T cells to cell adhesion molecules and/or inhibits beta integrin complex activation.

6. The method of claim 3, wherein the anti-layilin antibody is a bispecific antibody.

7. The method of claim 6, wherein a first variable domain of the bispecific antibody binds to layilin protein and a second variable domain of the bispecific antibody binds to an antigen expressed on the CD8+ T cells.

8. The method of claim 1, wherein the layilin-binding protein prevents or inhibits the binding of layilin to its natural ligand(s).

9. The method of claim 1, wherein the autoimmune disorder is in a tissue.

10. The method of claim 1, wherein the autoimmune disorder is an autoimmune skin disorder.

11. The method of claim 10, wherein the autoimmune skin disorder is selected from the group consisting of psoriasis, vitiligo, pemphigus vulgaris, pemphigus foliaceus, bullous pemphigoid, cicatricial pemphigoid, autoimmune alopecia, dermatitis herpetiformis, atopic dermatitis, and chronic autoimmune urticaria.

12. The method of claim 1, wherein the autoimmune disorder is an autoimmune lung disorder.

13. The method of claim 12, wherein the autoimmune lung disorder is lung scleroderma.

14. The method of claim 1, wherein the autoimmune disorder is an autoimmune gut disorder.

15. The method of claim 14, wherein the autoimmune gut disorder is selected from the group consisting of Crohn's disease, ulcerative colitis, and celiac disease.

16-19. (canceled)

20. A method for treating cancer in a subject in need thereof, comprising administering to the subject a modified CD8+ T cell having an increased layilin expression relative to an unmodified CD8+ T cell.

21-37. (canceled)

38. A modified CART cell comprising an increased layilin expression relative to an unmodified T cell.

39-167. (canceled)


SEQUENCE LISTING (50 sequences)

SEQ ID NO: 1 - protein, 382 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 2 - protein, 374 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 3 - protein, 262 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRQKTGAARP

SEQ ID NO: 4 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVQLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 5 - protein, 1170 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 6 - protein, 382 aa, Homo sapiens; identical to SEQ ID NO: 1

SEQ ID NO: 7 - protein, 374 aa, Homo sapiens; identical to SEQ ID NO: 2

SEQ ID NO: 8 - protein, 229 aa, Homo sapiens
MVTSGLGSGGVRRNKAIAQPARTFMLGLMAAYHNLEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 9 - DNA, 20 bp, Artificial Sequence
  Description of Artificial Sequence Synthetic oligonucleotide
ggtcatgtaccatcagccat

SEQ ID NO: 10 - DNA, 19 bp, Artificial Sequence
  Description of Artificial Sequence Synthetic oligonucleotide
ggttcttgactaccgtaat

SEQ ID NO: 11 - DNA, 21 bp, Artificial Sequence
  Description of Artificial Sequence Synthetic primer
ttctggtgcttgtctcactga

SEQ ID NO: 12 - DNA, 22 bp, Artificial Sequence
  Description of Artificial Sequence Synthetic primer
cagtatgttcggcttcccattc

SEQ ID NO: 13 - protein, 10 aa, Artificial Sequence
  Description of Artificial Sequence Synthetic peptide
AASRGDKLTF

SEQ ID NO: 14 - protein, 11 aa, Artificial Sequence
  Description of Artificial Sequence Synthetic peptide
CASRGGHEQYF

SEQ ID NO: 15 - protein, 12 aa, Artificial Sequence
  Description of Artificial Sequence Synthetic peptide
AERTEGNNRLAF

SEQ ID NO: 16 - protein, 14 aa, Artificial Sequence
  Description of Artificial Sequence Synthetic peptide
CASSSGQVNQPQHF

SEQ ID NO: 17 - protein, 14 aa, Artificial Sequence
  Description of Artificial Sequence Synthetic peptide
ALSDGTSGTYKYIF

SEQ ID NO: 18 - protein, 15 aa, Artificial Sequence
  Description of Artificial Sequence Synthetic peptide
CASSRDRGMNTEAFF

SEQ ID NO: 19 - protein, 11 aa, Artificial Sequence
  Description of Artificial Sequence Synthetic peptide
ALSLNDYKLSF

SEQ ID NO: 20 - protein, 15 aa, Artificial Sequence
  Description of Artificial Sequence Synthetic peptide
CASRLEEGAGGEQFF

SEQ ID NO: 21 - protein, 15 aa, Artificial Sequence
  Description of Artificial Sequence Synthetic peptide
CATRARGGPYNEQFF

SEQ ID NO: 22 - protein, 15 aa, Artificial Sequence
  Description of Artificial Sequence Synthetic peptide
AMRVNSGGSNYKLTF

SEQ ID NO: 23 - protein, 11 aa, Artificial Sequence
  Description of Artificial Sequence Synthetic peptide
CASRRVETQYF

SEQ ID NO: 24 - protein, 10 aa, Artificial Sequence
  Description of Artificial Sequence Synthetic peptide
AVKGFQKLVF

SEQ ID NO: 25 - protein, 11 aa, Artificial Sequence
  Description of Artificial Sequence Synthetic peptide
CSARGGAEAFF

SEQ ID NO: 26 - protein, 9 aa, Artificial Sequence
  Description of Artificial Sequence Synthetic peptide
AVKGQKLLF

SEQ ID NO: 27 - protein, 14 aa, Artificial Sequence
  Description of Artificial Sequence Synthetic peptide
CASSWLGGDETQYF

SEQ ID NO: 28 - protein, 9 aa, Artificial Sequence
  Description of Artificial Sequence Synthetic peptide
AVKSDKLIF

SEQ ID NO: 29 - protein, 17 aa, Artificial Sequence
  Description of Artificial Sequence Synthetic peptide
CASSYRPPGGIEDTQYF

SEQ ID NO: 30 - protein, 12 aa, Artificial Sequence
  Description of Artificial Sequence Synthetic peptide
AVLPTYGQNFVF

SEQ ID NO: 31 - protein, 12 aa, Artificial Sequence
  Description of Artificial Sequence Synthetic peptide
CASSSSGREQYF

SEQ ID NO: 32 - protein, 12 aa, Artificial Sequence
  Description of Artificial Sequence Synthetic peptide
AVMDTGRRALTF

SEQ ID NO: 33 - protein, 13 aa, Artificial Sequence
  Description of Artificial Sequence Synthetic peptide
CASSLGGSDEQYF

SEQ ID NO: 34 - protein, 11 aa, Artificial Sequence
  Description of Artificial Sequence Synthetic peptide
AVQSSAYKYIF

SEQ ID NO: 35 - protein, 12 aa, Artificial Sequence
  Description of Artificial Sequence Synthetic peptide
CASRGGGNTQYF

SEQ ID NO: 36 - protein, 12 aa, Artificial Sequence
  Description of Artificial Sequence Synthetic peptide
AVSEVYGNKLVF

SEQ ID NO: 37 - protein, 16 aa, Artificial Sequence
  Description of Artificial Sequence Synthetic peptide
CASSQDLFVGGETQYF

SEQ ID NO: 38 - protein, 10 aa, Artificial Sequence
  Description of Artificial Sequence Synthetic peptide
AVSPGYALNF

SEQ ID NO: 39 - protein, 15 aa, Artificial Sequence
  Description of Artificial Sequence Synthetic peptide
CASSRKGKGYNEQFF

SEQ ID NO: 40 - protein, 14 aa, Artificial Sequence
  Description of Artificial Sequence Synthetic peptide
LVGDGAGNNRKLIW

SEQ ID NO: 41 - protein, 16 aa, Artificial Sequence
  Description of Artificial Sequence Synthetic peptide
CASSPPRGSMNTEAFF

SEQ ID NO: 42 - protein, 10 aa, Artificial Sequence
  Description of Artificial Sequence Synthetic peptide
VVRGYQKVTF

SEQ ID NO: 43 - protein, 15 aa, Artificial Sequence
  Description of Artificial Sequence Synthetic peptide
CASSSPVSSNYGYTF

SEQ ID NO: 44 - protein, 13 aa, Artificial Sequence
  Description of Artificial Sequence Synthetic peptide
VVRIGGSQGNLIF

SEQ ID NO: 45 - protein, 15 aa, Artificial Sequence
  Description of Artificial Sequence Synthetic peptide
CASGSASGGPVTQYF

SEQ ID NO: 46 - protein, 8 aa, Artificial Sequence
  Description of Artificial Sequence Synthetic peptide
ASNYQLIW

SEQ ID NO: 47 - protein, 12 aa, Artificial Sequence
  Description of Artificial Sequence Synthetic peptide
AVWVNAGNMLTF

SEQ ID NO: 48 - protein, 12 aa, Artificial Sequence
  Description of Artificial Sequence Synthetic peptide
CASSLVGHEQFF

SEQ ID NO: 49 - DNA, 49 bp, Mus sp.
tggcggtgctgctggccaaaccgagggattcgaagggtcgcctgctgag

SEQ ID NO: 50 - DNA, 50 bp, Mus sp.
accacccggcatcgggggctcgtacatgttccagtggaatgatgaccggt